(19) United States
(12) Patent Application Publication
(10) Pub. No.: US 2026/0071239 A1
(43) Pub. Date: Mar. 12, 2026

(54) COMPOSITIONS AND METHODS FOR IMPROVED CELL CULTURE EFFICIENCY

(71) Applicant: Amyris, Inc., Emeryville, CA (US)

(72) Inventors: Joshua LENG, Emeryville, CA (US); Bradon FRIEDRIKSON, Emeryville, CA (US); Paul W. HILL, Emeryville, CA (US); Weiyin WU, Emeryville, CA (US)

(21) Appl. No.: 18/873,143

(22) PCT Filed: Jun. 9, 2023
(86) PCT No.: PCT/US2023/024986

§ 371 Date: Dec. 9, 2024

Related U.S. Application Data
(60) Provisional application No. 63/351,159, filed on Jun. 10, 2022.

(51) Int. Cl. C12P 5/00

(52) U.S. Cl. CPC C12P 5/007

(57) ABSTRACT

The present disclosure provides compositions and methods for producing a cell culture product in a cell culture composition, particularly for cell culture product s that impair the growth and/or function of the host cells in which they are biosynthesized, such as isoprenoids or terpenes.

### BACKGROUND OF THE INVENTION

Isoprenoids are ubiquitous in nature. They comprise a diverse family of over 40,000 individual molecules, many of which are vital to living organisms. Isoprenoids serve to maintain cellular fluidity, electron transport, and other metabolic functions. A vast number of natural and synthetic isoprenoids are useful as pharmaceuticals, cosmetics, perfumes, pigments and colorants, fungicides, antiseptics, nutraceuticals, and fine chemical intermediates. An isoprenoid product is typically composed of repeating five carbon isopentenyl diphosphate (IPP) units, although irregular isoprenoids and polyterpenes have been reported.

Isoprenoids, including terpenes, are commonly used in the flavor and fragrance industry as well as other industries. However, many isoprenoids, particularly monoterpenes, are inhibitory to the host cells that are capable of synthesizing them given that as the concentration of product accumulates, the inhibitory effect to the cells also increases. Moreover, optimal biomass is desirably achieved based on cell culture conditions such as sugar concentration and supply and oxygen supply in order to maximize cell culture yield and productivity. Accordingly, producing isoprenoids by way of cell culture in a host cell (e.g., a yeast cell) is particularly challenging. There remains a need for fermentative compositions and methods capable of effectively producing isoprenoids with high yield and efficiency.

### SUMMARY OF THE INVENTION

The present disclosure provides compositions and methods for producing a cell culture product (e.g., fermentation product) in a cell culture composition (e.g., fermentation composition), particularly from host cells capable of synthesizing a cell culture product (e.g., an isoprenoid or a terpene, among others). The compositions and methods described herein address the problem of how to produce compounds using a host cell when the compound that is produced is inhibitory to the host cell, when cell biomass is low (e.g., poor growing strain), or when feed sugar concentrations are low (e.g., ligno-cellulosic feedstocks). As host cells are cultured in a cell culture composition to produce such a compound, the compound's increase in concentration may cause the host cells to have decreased productivities and low yields of the cell culture composition. Given the inhibitory nature of the desired compounds, producing them from host cell has presented significant challenges. In another instance, the compositions and methods described herein are useful in retaining cell biomass, which is important in strains that are poor growing, by separating host cells from a cell culture product and returning the host cells to a vessel as opposed to continuously removing them from the cell culture composition. Furthermore, the methods and compositions described herein are useful in allowing for more dilute feedstocks by returning the cells to the vessel. Finally, products that are susceptible to chemical modification in the culture medium may degrade and/or oxidize over the course of several days in the cell culture environment, and by sequestering the product from the cell culture composition, product degradation and/or oxidation may be prevented or reduced, thereby improving yield and/or purity.

The present discourse is based, in part, on the surprising discovery that greater yield and productivity of a cell culture product can be achieved by contacting a cell culture composition with a water-immiscible solvent. This can cause the cell culture product to extract into the water-immiscible solvent, removing the cell culture product from the host cells to which the product is inhibitory. To further improve yield and productivity, any host cells that are removed during this extraction may be recycled back into the cell culture composition. The sections that follow provide a description of the compositions and methods that can be used to produce an inhibitory cell culture product in host cells (e.g., yeast cells) guided by this discovery.

In a first aspect, the disclosure provides a method of producing a cell culture product including: (a) culturing, in a vessel, a population of host cells capable of producing the cell culture product in an aqueous-phase culture medium and under conditions suitable for the host cells to produce the cell culture product, thereby producing the cell culture product and forming a cell culture composition; (b) contacting the cell culture composition with a water-immiscible solvent, thereby (i) forming a mixture and (ii) partitioning the cell culture product between the cell culture composition and the water-immiscible solvent; (c) selecting a portion of the mixture resulting from (b); (d) from the portion of the mixture selected in (c), separating a plurality of the host cells from the cell culture composition; (e) returning the plurality of the host cells to the vessel; and (f) from the portion of the mixture selected in (c), recovering the cell culture product from the water-immiscible solvent.

In another aspect, the disclosure provides a method of isolating a cell culture product from a cell culture composition. The method includes (a) providing, in a vessel, a cell culture composition that has been produced by culturing a population of host cells capable of producing the cell culture product in an aqueous-phase culture medium and under conditions suitable for the host cells to produce the cell culture product; (b) contacting the cell culture composition with a water-immiscible solvent, thereby (i) forming a mixture and (ii) partitioning the cell culture product between the cell culture composition and the water-immiscible solvent; (c) selecting a portion of the mixture resulting from (b); (d) from the portion of the mixture selected in (c), separating a plurality of the host cells from the cell culture composition; (e) returning the plurality of the host cells to the vessel; and (f) recovering the cell culture product from the water-immiscible solvent resulting from (c).

In another aspect, the disclosure provides a method of isolating a cell culture product from a cell culture composition. The method includes (a) providing, in a vessel, a mixture comprising (i) a water-immiscible solvent and (ii) a cell culture composition that has been produced by culturing a population of host cells capable of producing the cell culture product in an aqueous-phase culture medium and under conditions suitable for the host cells to produce the cell culture product, wherein the cell culture product is partitioned between the cell culture composition and the water-immiscible solvent; (b) selecting a portion of the mixture; (c) from the portion of the mixture selected in (b), separating a plurality of the host cells from the cell culture composition; (d) returning the plurality of the host cells to the vessel; and (e) recovering the cell culture product from the water-immiscible solvent resulting from (c).

In some embodiments, the method includes repeating steps (a)-(f) a plurality of times, optionally wherein the method includes repeating steps (a)-(f) continuously. In some embodiments, the method includes repeating steps (a)-(f) a plurality of times discontinuously. In some embodiments, the method including repeating steps (b)-(f) a plurality of times, optionally wherein the method includes repeating steps (b)-(f) continuously. In some embodiments, the method including repeating steps (b)-(f) a plurality of times discontinuously. In some embodiments, the method including repeating steps (b)-(e) a plurality of times, optionally wherein the method includes repeating steps (b)-(e) continuously. In some embodiments, the method including repeating steps (b)-(e) a plurality of times discontinuously.

In some embodiments, the method further includes adding fresh water-immiscible solvent to the vessel of cell culture composition following the selecting step. In some embodiments, the plurality of the host cells is separated from the water-immiscible solvent by way of a gravity separation process. In some embodiments, the gravity separation process includes cell sedimentation.

In another aspect, the disclosure features a method of producing a cell culture composition by (a) culturing, in a vessel, a population of host cells capable of producing the cell culture product in an aqueous-phase culture medium and under conditions suitable for the host cells to produce the cell culture product, thereby producing the cell culture product and forming a cell culture composition; (b) selecting a portion of the cell culture composition; (c) separating a plurality of the host cells from the portion of the cell culture composition selected in (b); (d) returning the plurality of the host cells to the vessel; and (e) recovering the cell culture product from the portion of the cell culture composition selected in (b). In some embodiments, the cell culture product includes (i) a compound that is water-immiscible, optionally a compound having a log(D) value of from about 1 to about 15; (ii) a compound that is inhibitory to the host cells; and/or (iii) an isoprenoid or terpene, optionally wherein the cell culture product comprises a monoterpene. In some embodiments, the plurality of the host cells is separated from the cell culture composition by way of a gravity separation process. In some embodiments, the gravity separation process comprises cell sedimentation. In some embodiments, the cell sedimentation is achieved using a gravity settling device.

In some embodiments of any of the above aspects or embodiments of the disclosure, the cell sedimentation is achieved using a gravity settling device. In some embodiments, the gravity settling device includes: (i) an inlet tube that is in fluid communication with, and that receives the portion of the mixture from, the vessel; (ii) a settling chamber that is in fluid communication with, and that receives the portion of the mixture from, the inlet tube, wherein the settling chamber has an incline angle of greater than 0° and less than, or equal to, 90°, optionally wherein the settling chamber has an incline angle of from about 25° to about 75°, optionally wherein the settling chamber has an incline angle of from about 35° to about 55°, optionally wherein the settling chamber has an incline angle of about 45°; (iii) an outlet at the bottom of the settling chamber that is in fluid communication with the vessel; (iv) an outlet at the top of the settling chamber that is in fluid communication with an effluent vessel; and, optionally, (v) an overflow outlet at the top of the inlet tube that is in fluid communication with the effluent vessel, whereby upon introduction into the inlet tube of an excess of the mixture that exceeds the volume of the settling chamber, the excess mixture flows through the overflow outlet and into the effluent vessel.

In some embodiments, the cell sedimentation includes: (i) introducing the portion of the mixture into the inlet tube; (ii) allowing the plurality of the host cells to flow to the bottom of the settling chamber and, subsequently, to return to the vessel through the outlet at the bottom of the settling chamber; (iii) removing the water-immiscible solvent from the settling chamber through the outlet at the top of the settling chamber and delivering the water-immiscible solvent to the effluent bottle; and, optionally, (iv) removing any excess mixture that exceeds the volume of the settling chamber through the overflow outlet and delivering the excess mixture to the effluent vessel.

In some embodiments, the gravity settling device further includes a bubble trap. In some embodiments, the bubble trap and the settling chamber are joined at an angle of between about 60° and 120° (e.g., an angle of about 60°, 65°, 70°, 75°, 80°, 85°, 90°, 95°, 100°, 105°, 110°, 115°, or 120°). In some embodiments, the bubble trap and the settling chamber are joined at an angle of about 45°. In some embodiments, the bubble trap is located between the vessel and the settling chamber. In some embodiments, the bubble trap is joined to the settling chamber by way of a tube, wherein the settling chamber is between about 60° and 120° (e.g., an angle of about 60°, 65°, 70°, 75°, 80°, 85°, 90°, 95°, 100°, 105°, 110°, 115°, or 120°) from vertical.

In some embodiments, the portion of the mixture is delivered to the inlet tube by way of a pump. In some embodiments, the water-immiscible solvent is removed from the settling chamber and delivered through the outlet at the top of the settling chamber to the effluent bottle by way of a pump.

In some embodiments, the method includes introducing a carbon source into the vessel. In some embodiments, the carbon source is continuously introduced into the vessel. In some embodiments, the carbon source is introduced into the vessel by way of a pump.

In some embodiments, the method includes oxygenating the cell culture composition. In some embodiments, the cell culture composition is oxygenated by way of delivering compressed air into the vessel. In some embodiments, the method including mixing the cell culture composition by way of an impeller.

In some embodiments, the cell culture product is a water-immiscible compound. In some embodiments, the cell culture product is a compound that is inhibitory to the host cells. In some embodiments, the cell culture product is a terpene. In some embodiments, the terpene is a C₅-C₄₀ terpene (e.g., a C₅ terpene, C₁₀ terpene, C₁₅ terpene, C₂₀ terpene, C₂₅ terpene, C₃₀ terpene, C₃₅ terpene, or C₄₀ terpene). In some embodiments, the terpene is a C₅-C₂₀ terpene (e.g., C₅ terpene, C₆ terpene, C₇ terpene, C₈ terpene, C₉ terpene, C₁₀ terpene, C₁₁ terpene, C₁₂ terpene, C₁₃ terpene, C₁₄ terpene, C₁₅ terpene, C₁₆ terpene, C₁₇ terpene, C₁₈ terpene, C₁₉ terpene, or C₂₀ terpene). In some embodiments, the terpene is a C₁₀-C₁₅ terpene (e.g., C₁₀ terpene, C terpene, C₁₂ terpene, C₁₃ terpene, C₁₄ terpene, or C₁₅ terpene). In some embodiments, the terpene is a hemiterpene, monoterpene, sesquiterpene, diterpene, sesterterpene, triterpene, tetraterpene, or polyterpene. In some embodiments, the terpene is a monoterpene.

In some embodiments, the cell culture product is an isoprenoid. In some embodiments, the isoprenoid is a C₅-C₄₀ isoprenoid (e.g., a C₅ isoprenoid, C₁₀ isoprenoid, C₁₅ isoprenoid, C₂₀ isoprenoid, C₂₅ isoprenoid, C₃₀ isoprenoid, C₃₅ isoprenoid, or C₄₀ isoprenoid). In some embodiments, the isoprenoid is a C₅-C₂₀ isoprenoid (e.g., C₅ isoprenoid, C₆ isoprenoid, C₇ isoprenoid, C₈ isoprenoid, C₉ isoprenoid, C₁₀ isoprenoid, C₁₁ isoprenoid, C₁₂ isoprenoid, C₁₃ isoprenoid, C₁₄ isoprenoid, C₁₅ isoprenoid, C₁₆ isoprenoid, C₁₇ isoprenoid, C₁₈ isoprenoid, C₁₉ isoprenoid, or C₂₀ isoprenoid). In some embodiments, the isoprenoid is a C₁₀-C₁₅ isoprenoid (e.g., C₁₀ isoprenoid, C₁₁ isoprenoid, C₁₂ isoprenoid, C₁₃ isoprenoid, C₁₄ isoprenoid, or C₁₅ isoprenoid). In some embodiments, the isoprenoid is a hemiterpenoid, monoterpenoid, sesquiterpenoid, diterpenoid, sesterterpenoid, triterpenoid, tetraterpenoid, or polyterpenoid. “In other embodiments, the isoprenoid is a C15 isoprenoid. These compounds are derived from three isoprene units and are also called sesquiterpenoids. Illustrative examples of sesquiterpenoids are periplanone B, gingkolide B, amorphadiene, artemisinin, artemisinic acid, valencene, nootkatone, epicedrol, epiaristolochene, farnesol, gossypol, sanonin, periplanone, forskolin, and patchoulol, which is also known as patchouli alcohol. In some embodiments, the isoprenoid is a monoterpenoid. In some embodiments, the cell culture product is the cell culture product is abietadiene, anethole, amorphadiene, carene, carvacrol, creosol, cuminaldehyde, eugenol, α-farnesene, β-farnesene, farnesol, geraniol, geranylgeraniol, hinokitiol, isoprene, linalool, limonene, myrcene, nerolidol, ocimene, patchoulol, β-pinene, sabinene, γ-terpinene, terpinolene, menthol, eucalyptol, citronellol, citronellal, valencene, or a salvinorin. In some embodiments, the cell culture product is β-farnesene. In some embodiments, the cell culture product is myrcene. In some embodiments, the cell culture product is pinene. In some embodiments, the cell culture product is limonene. In some embodiments, the cell culture product is menthol. In some embodiments, the cell culture product is citronellal. In some embodiments, the cell culture product is citronellol. In some embodiments, the cell culture product is farnesol. In some embodiments, the cell culture product is terpinene. In some embodiments, the cell culture product is terpinolene. In some embodiments, the cell culture product is geraniol. In some embodiments, the cell culture product is linalool. In some embodiments, the cell culture product is hinokitiol. In some embodiments, the cell culture product is carvacrol. In some embodiments, the cell culture product is anethole. In some embodiments, the cell culture product is cuminaldehyde. In some embodiments, the cell culture product is a salvinorin. For example, the salvinorin may be salvinorin A, salvinorin B, salvinorin C, salvinorin D, salvinorin E, salvinorin F, salvinorin G, salvinorin H, salvinorin I, 17α-salvinorin J, or 17β-salvinorin J. In some embodiments, the cell culture product is eugenol. In some embodiments, the cell culture product is creosol.

In some embodiments, the cell culture product is produced from one or more inhibitory compounds.

In some embodiments, the cell culture product has a minimum inhibitory concentration (MIC) in the host cells of from about 0.1 mM to about 5 mM (e.g., about 0.1 mM to about 4 mM, about 0.1 mM to about 3 mM, about 0.1 mM to about 2 mM, about 0.1 mM to about 1 mM, about 0.1 mM to about 0.5 mM, about 0.5 mM to about 5 mM, about 1 mM to about 5 mM, about 2 mM to about 5 mM, about 3 mM to about 5 mM, or about 4 mM to about 5 mM). In some embodiments, the MIC in the host cells is greater than 5 mM. In some embodiments, the cell culture product has a MIC in the host cells of from about 0.1 mM to about 2.5 mM, optionally wherein the cell culture product has a MIC in the host cells of about 0.1 mM, about 0.15 mM, about 0.2 mM, about 0.25 mM, about 0.3 mM, about 0.35 mM, about 0.4 mM, about 0.45 mM, about 0.5 mM, about 0.55 mM, about 0.6 mM, about 0.65 mM, about 0.7 mM, about 0.75 mM, about 0.8 mM, about 0.85 mM, about 0.9 mM, about 0.95 mM, about 1 mM, about 1.25 mM, about 1.3 mM, about 1.35 mM, about 1.4 mM, about 1.45 mM, about 1.5 mM, about 1.55 mM, about 1.6 mM, about 1.65 mM, about 1.7 mM, about 1.75 mM, about 1.8 mM, about 1.85 mM, about 1.9 mM, about 1.95 mM, about 2 mM, about 2.25 mM, about 2.3 mM, about 2.35 mM, about 2.4 mM, about 2.45 mM, or about 2.5 mM.

In some embodiments, the water-immiscible solvent has a log(K_{d}) value of from about 1 to about 15 (e.g., about 0.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.22, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, or 15)), wherein the K_{d} is the partition coefficient for the cell culture product between the water-immiscible solvent and the cell culture composition. In some embodiments, the water-immiscible solvent has a log(K_{d}) value of from about 2 to about 3, optionally wherein the water-immiscible solvent has a log(K_{d}) value of about 2, about 2.1, about 2.2, about 2.3, about 2.4, about 2.5, about 2.6, about 2.7, about 2.8, about 2.9, or about 3.

In some embodiments, the cell culture product has a log(D) value of from about 1 to about 15 (e.g., about 1.1, 1.2, 1.3, 1.4, 1.5, 1.6, 1.7, 1.8, 1.9, 2, 2.1, 2.2, 2.3, 2.4, 2.5, 2.6, 2.7 2.8, 2.9, 3.1, 3.2, 3.3, 3.4, 3.5, 3.6, 3.7, 3.8, 3.9, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.22, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, or 15).

In some embodiments, the water-immiscible solvent is an alcohol. In some embodiments, the water-immiscible solvent is a C₁₀-C₂₀ alcohol (e.g., a C₁₀ alcohol, C alcohol, C₁₂ alcohol, C₁₃ alcohol, C₁₄ alcohol, C₁₅ alcohol, C₁₆ alcohol, C₁₇ alcohol, C₁₈ alcohol, C₁₉ alcohol, or C₂₀ alcohol). In some embodiments, the water-immiscible solvent is a C₁₂-C₁₈ alcohol (e.g., a C₁₂ alcohol, C₁₃ alcohol, C₁₄ alcohol, C₁₅ alcohol, C₁₆ alcohol, C₁₇ alcohol, or C₁₈ alcohol). In some embodiments, the water-immiscible solvent is a vegetable oil. In some embodiments, the water-immiscible solvent is corn oil, sunflower oil, soybean oil, mineral oil, polyalphaolefin, dodecane, hexadecane, oleyl alcohol, butyl oleate, dibutyl phthalate, dodecanol, dioctyl phthalate, farnesene, or isopropyl myristate.

In some embodiments, the vessel has a capacity of from about 0.5 L to about 600,000 L (e.g., about 0.5 L to about 500,000 L, about 0.5 L to about 400,000 L, about 0.5 L to about 300,000 L, about 0.5 L to about 200,000 L, about 0.5 L to about 100,000 L, about 0.5 L to about 80,000 L, about 0.5 L to about 60,000 L, about 0.5 L to about 40,000, about 0.5 L to about 20,000 L, about 0.5 L to about 1,000 L, about 1,000 L to about 600,000 L, about 20,000 L to about 600,000 L, about 40,000 L to about 600,000 L, about 60,000 L to about 600,000 L, about 80,000 L to about 600,000 L, about 100,000 L to about 600,000 L, about 200,000 L to about 600,000 L, about 300,000 L to about 600,000 L, about 400,000 L to about 600,000 L, or about 500,000 L to about 600,000 L). In some embodiments, the vessel has a capacity of from about 0.5 L to about 1,000 L (e.g., about 0.5 L to about 100 L, about 0.5 L to about 200 L, about 0.5 L to about 300 L, about 0.5 L to about 400 L, about 0.5 L to about 500 L, about 0.5 L to about 600 L, about 0.5 L to about 700 L, about 0.5 L to about 800 L, about 0.5 L to about 900 L, about 0.5 L to about 1,000 L, about 100 L to about 1,000 L, about 200 L to about 1,000 L, about 300 L to about 1,000 L, about 400 L to about 1,000 L, about 500 L to about 1,000 L, about 600 L to about 1,000 L, about 700 L to about 1,000 L, about 800 L to about 1,000 L, or about 900 L to about 1,000 L).

In some embodiments, the carbon source is introduced into the vessel at a rate of from about 0.1 g total reducing sugar (TRS)/L/hour to about 30 g TRS/L/hour (e.g., about 0.1 g TRS/L/hour to about 1 g TRS/L/hour, about 0.1 g TRS/L/hour to about 2 g TRS/L/hour, about 0.1 g TRS/L/hour to about 3 g TRS/L/hour, about 0.1 g TRS/L/hour to about 4 g TRS/L/hour, about 0.1 g TRS/L/hour to about 5 g TRS/L/hour, about 0.1 g TRS/L/hour to about 6 g TRS/L/hour, about 0.1 g TRS/L/hour to about 7 g TRS/L/hour, about 0.1 g TRS/L/hour to about 8 g TRS/L/hour, about 0.1 g TRS/L/hour to about 9 g TRS/L/hour, about 0.1 g TRS/L/hour to about 10 g TRS/L/hour, about 0.1 g TRS/L/hour to about 11 g TRS/L/hour, about 0.1 g TRS/L/hour to about 12 g TRS/L/hour, about 0.1 g TRS/L/hour to about 13 g TRS/L/hour, about 0.1 g TRS/L/hour to about 14 g TRS/L/hour, about 0.1 g TRS/L/hour to about 15 g TRS/L/hour, about 0.1 g TRS/L/hour to about 20 g TRS/L/hour, about 0.1 g TRS/L/hour to about 25 g TRS/L/hour, about 0.1 g TRS/L/hour to about 30 g TRS/L/hour, about 1 g TRS/L/hour to about 30 g TRS/L/hour, about 2 g TRS/L/hour to about 30 g TRS/L/hour, about 3 g TRS/L/hour to about 30 g TRS/L/hour, about 4 g TRS/L/hour to about 30 g TRS/L/hour, about 5 g TRS/L/hour to about 30 g TRS/L/hour, about 6 g TRS/L/hour to about 30 g TRS/L/hour, about 7 g TRS/L/hour to about 30 g TRS/L/hour, about 8 g TRS/L/hour to about 30 g TRS/L/hour, about 9 g TRS/L/hour to about 30 g TRS/L/hour, about 10 g TRS/L/hour to about 30 g TRS/L/hour, about 11 g TRS/L/hour to about 30 g TRS/L/hour, about 12 g TRS/L/hour to about 30 g TRS/L/hour, about 13 g TRS/L/hour to about 30 g TRS/L/hour, about 14 g TRS/L/hour to about 30 g TRS/L/hour, about 15 g TRS/L/hour to about 30 g TRS/L/hour, about 20 g TRS/L/hour to about 30 g TRS/L/hour, or about 25 g TRS/L/hour to about 30 g TRS/L/hour). In some embodiments, the carbon source is introduced into the vessel at a rate of from about 1 g TRS/L/hour to about 15 g TRS/L/hour (e.g., about 1 g TRS/L/hour to about 2 g TRS/L/hour, about 1 g TRS/L/hour to about 3 g TRS/L/hour, about 1 g TRS/L/hour to about 4 g TRS/L/hour, about 1 g TRS/L/hour to about 5 g TRS/L/hour, about 1 g TRS/L/hour to about 6 g TRS/L/hour, about 1 g TRS/L/hour to about 7 g TRS/L/hour, about 1 g TRS/L/hour to about 8 g TRS/L/hour, about 1 g TRS/L/hour to about 9 g TRS/L/hour, about 1 g TRS/L/hour to about 10 g TRS/L/hour, about 1 g TRS/L/hour to about 11 g TRS/L/hour, about 1 g TRS/L/hour to about 12 g TRS/L/hour, about 1 g TRS/L/hour to about 13 g TRS/L/hour, about 1 g TRS/L/hour to about 14 g TRS/L/hour, about 1 g TRS/L/hour to about 15 g TRS/L/hour, about 2 g TRS/L/hour to about 15 g TRS/L/hour, about 3 g TRS/L/hour to about 15 g TRS/L/hour, about 4 g TRS/L/hour to about 15 g TRS/L/hour, about 5 g TRS/L/hour to about 15 g TRS/L/hour, about 6 g TRS/L/hour to about 15 g TRS/L/hour, about 7 g TRS/L/hour to about 15 g TRS/L/hour, about 8 g TRS/L/hour to about 15 g TRS/L/hour, about 9 g TRS/L/hour to about 15 g TRS/L/hour, about 10 g TRS/L/hour to about 15 g TRS/L/hour, about 11 g TRS/L/hour to about 15 g TRS/L/hour, about 12 g TRS/L/hour to about 15 g TRS/L/hour, about 13 g TRS/L/hour to about 15 g TRS/L/hour, or about 14 g TRS/L/hour to about 15 g TRS/L/hour).

In some embodiments, the carbon source introduced into the vessel has a concentration of from about 10% (w/v) to about 80% (w/v) of total reducing sugar (e.g., about 10% (w/v), 20% (w/v), 30% (w/v), 40% (w/v), 50% (w/v), 60% (w/v), 70% (w/v), or 80% (w/v)). In some embodiments, the carbon source introduced into the vessel has a concentration of about 30% (w/v) of total reducing sugar. In some embodiments, the carbon source has introduced into the vessel has a concentration of about 60% (w/v) of total reducing sugar.

In some embodiments, the host cells in the cell culture composition consume oxygen at a rate of from about 25 mmol/L/hr to about 250 mmol/L/hr (e.g., about 25 mmol/L/hr to about 225 mmol/L/hr, about 25 mmol/L/hr to about 200 mmol/L/hr, about 25 mmol/L/hr to about 175 mmol/L/hr, about 25 mmol/L/hr to about 150 mmol/L/hr, about 25 mmol/L/hr to about 125 mmol/L/hr, about 25 mmol/L/hr to about 100 mmol/L/hr, about 25 mmol/L/hr to about 75 mmol/L/hr, about 75 mmol/L/hr to about 250 mmol/L/hr, about 100 mmol/L/hr to about 250 mmol/L/hr, about 125 mmol/L/hr to about 250 mmol/L/hr, about 150 mmol/L/hr to about 250 mmol/L/hr, about 175 mmol/L/hr to about 250 mmol/L/hr, about 200 mmol/L/hr to about 250 mmol/L/hr, or about 225 mmol/L/hr to about 250 mmol/L/hr). In some embodiments, the host cells in the cell culture composition consume oxygen at a rate of from about 90 mmol/L/hr to about 130 mmol/L/hr (e.g., about 90 mmol/L/hr to about 120 mmol/L/hr, about 90 mmol/L/hr to about 110 mmol/L/hr, about 90 mmol/L/hr to about 100 mmol/L/hr, about 100 mmol/L/hr to about 130 mmol/L/hr, about 110 mmol/L/hr to about 130 mmol/L/hr, or about 120 mmol/L/hr to about 130 mmol/L/hr). In some embodiments, the host cells in the cell culture composition consume oxygen at a rate of from about 110 mmol/L/hr.

In some embodiments, the host cells are sedimented at a rate of about 0.003 mm/min or greater, optionally wherein the host cells are sedimented at a rate of from about 0.003 mm/min to about 0.5 mm/min (e.g., about 0.003 mm/min to about 0.4 mm/min, about 0.003 mm/min to about 0.3 mm/min, about 0.003 mm/min to about 0.2 mm/min, about 0.003 mm/min to about 0.1 mm/min, about 0.003 mm/min to about 0.05 mm/min, about 0.003 mm/min to about 0.01 mm/min, about 0.003 mm/min to about 0.005 mm/min, about 0.005 mm/min to about 0.5 mm/min, about 0.01 mm/min to about 0.5 mm/min, about 0.05 mm/min to about 0.5 mm/min, about 0.1 mm/min to about 0.5 mm/min about 0.2 mm/min to about 0.5 mm/min, about 0.3 mm/min to about 0.5 mm/min, or about 0.4 mm/min to about 0.5 mm/min).

In some embodiments, the plurality of the host cells is returned to the vessel at a rate of from about 1 ml/L/min to about 300 ml/L/min (e.g., about 1 ml/L/min to about 250 ml/L/min, about 1 ml/L/min to about 200 ml/L/min, about 1 ml/L/min to about 150 ml/L/min, about 1 ml/L/min to about 100 ml/L/min, about 1 ml/L/min to about 50 ml/L/min, about 1 ml/L/min to about 25 ml/L/min, about 2 ml/L/min to about 300 ml/L/min, about 3 ml/L/min to about 300 ml/L/min, about 4 ml/L/min to about 300 ml/L/min, about 10 mL/L/min to about 300 mL/L/min, about 50 mL/L/min to about 300 mL/L/min, about 100 mL/L/min to about 300 mL/L/min, about 150 mL/L/min to about 300 mL/L/min, about 200 mL/L/min to about 300 mL/L/min, or about 250 mL/L/min to about 300 mL/L/min).

In some embodiments, the water-immiscible solvent is added to the cell culture composition to a final concentration of water-immiscible solvent of from about 0.5% (v/v) to about 50% (v/v) (e.g., about 0.5% (v/v) to about 40% (v/v), about 0.5% (v/v) to about 10% (v/v), about 1% (v/v) to about 10% (v/v), about 1% (v/v) to about 20% (v/v), about 1% (v/v) to about 30% (v/v), about 1% to about 40% (v/v), about 40% (v/v) to about 50% (v/v), about 30% (v/v) to about 50% (v/v), about 20% (v/v) to about 50% (v/v), or about 10% (v/v) to about 50% (v/v)). In some embodiments, the water-immiscible solvent is added to the cell culture composition to a final concentration of water-immiscible solvent of from about 5% (v/v) to about 25% (v/v) (e.g., about 5% (v/v), 6% (v/v), 7% (v/v), 8% (v/v), 9% (v/v), 10% (v/v), 11% (v/v), 12% (v/v), 13% (v/v), 14% (v/v), 15% (v/v), 16% (v/v), 17% (v/v), 18% (v/v), 19% (v/v), 20% (v/v), 21% (v/v), 22% (v/v), 23% (v/v), 24% (v/v), or 25% (v/v)).

In some embodiments, the host cells include one or more heterologous nucleic acids that each, independently, encode an enzyme of the 1-deoxy-D-xylulose 5-diphosphate (DXP) biosynthetic pathway. In some embodiments, the one or more heterologous nucleic acids encode one or more of a 1-deoxy-D-xylulose-5-phosphate synthase, a 1-deoxy-D-xylulose-5-phosphate reductoisomerase, a 4-diphosphocytidyl-2C-methyl-D-erythritol synthase, 4-diphosphocytidyl-2C-methyl-D-erythritol kinas-, a 2C-methyl-D-erythritol 2,4-cyclodiphosphate synthase, and/or 1-hydroxy-2-methyl-2-(E)-butenyl-4-diphosphate synthase, In some embodiments, the one or more heterologous nucleic acids encoding one or more enzymes of the DXP biosynthetic pathway are integrated into the genome of the host cell. In some embodiments, the one or more heterologous nucleic acids encoding one or more enzymes of the DXP biosynthetic pathway are present within one or more plasmids.

In some embodiments, the host cells include one or more heterologous nucleic acids that each, independently, encode an enzyme of the mevalonate (MEV) biosynthetic pathway. In some embodiments, the one or more heterologous nucleic acids encode one or more of an acetyl-CoA thiolase, an HMG-CoA synthase, an HMG-CoA reductase, a mevalonate kinase, a phosphomevalonate kinase, a mevalonate pyrophosphate decarboxylase, and/or an isopentenyl diphosphate (IPP):DMAPP isomerase. In some embodiments, the one or more heterologous nucleic acids encoding one or more enzymes of the MEV biosynthetic pathway are integrated into the genome of the host cell. In some embodiments, the one or more heterologous nucleic acids encoding one or more enzymes of the MEV biosynthetic pathway are present within one or more plasmids.

In some embodiments, the host cells include one or more heterologous nucleic acids that each, independently, encode an enzyme of the cannabinoid biosynthetic pathway. In some embodiments, the one or more heterologous nucleic acids encode one or more of an acetyl-CoA thiolase, an HMG-CoA synthase, an HMG-CoA reductase, a mevalonate kinase, a phosphomevalonate kinase, a mevalonate pyrophosphate decarboxylase, and an IPP:DMAPP isomerase. In some embodiments, the one or more heterologous nucleic acids encoding one or more enzymes of the cannabinoid biosynthetic pathway are integrated into the genome of the host cell. In some embodiments, the one or more heterologous nucleic acids encoding one or more enzymes of the cannabinoid biosynthetic pathway are present within one or more plasmids.

In some embodiments, the host cells are bacterial cells. In some embodiments, the host cells are fungal cells. In some embodiments, the host cells are E. coli, B. subtilis, Actinomyces, or Aspergillus. In some embodiments, the host cells are filamentous fungi. For example, the filamentous fungi may be Penicillium. In some embodiments, the host cells are yeast cells. In some embodiments, the yeast cells are Saccharomyces sp. cells or Kluveromyces sp. cells. In some embodiments, the yeast cells are Saccharomyces cerevisiae cells. In some embodiments, the yeast cells are Kluveromyces marxianus cells.

In some embodiments, the method results in biomass retention of at least 50% (e.g., at least 60%, 65%, 70%, 75%, 80%, 85%, 90%, 95%, or 99%). In some embodiments, the method results in performance enhancement of the host cells relative to a reference method in which a gravity settling device is not used to effect cell sedimentation. The performance enhancement may manifest in the form an increase in cell culture yield, cell culture productivity, and/or cell culture length. In some embodiments, the method results in performance enhancement of about 0.5% to about 500% (e.g., about 0.5% to about 400%, about 0.5% to about 300%, about 0.5% to about 200%, about 0.5% to about 100%, about 0.5% to about 50%, about 50% to about 500%, about 100% to about 500%, about 200% to about 500%, about 300% to about 500%, or about 400% to about 500%) relative to a reference method in which a gravity settling device is not used to effect cell sedimentation. In some embodiments, the method results in performance enhancement that manifests in the form of an increase in cell culture yield. In some embodiments, the method results in performance enhancement that manifests in the form of an increase in cell culture productivity. In some embodiments, the method results in performance enhancement that manifests in the form of an increase in cell culture length.

In some embodiments, any one of the methods described herein is performed aseptically.

In some embodiments, the cell culture product is a fermentation product. In some embodiments, the cell culture composition is a fermentation composition. In some embodiments, the vessel is a fermentation vessel.

In another aspect, the disclosure provides a composition including a cell culture product dissolved in a water-immiscible solvent, wherein the composition is obtained by a method including (a) culturing, in a vessel, a population of host cells capable of producing the cell culture product in an aqueous-phase culture medium and under conditions suitable for the host cells to produce the cell culture product, thereby producing a cell culture composition; (b) contacting the cell culture composition with a water-immiscible solvent, thereby (i) forming a mixture and (ii) partitioning the cell culture product between the cell culture composition and the water-immiscible solvent; (c) selecting from the vessel a portion of the mixture resulting from (b); (d) from the portion of the mixture selected in (c), separating a plurality of the host cells from the water-immiscible solvent; (e) returning the plurality of the host cells to the vessel; and (f) recovering the cell culture product from the water-immiscible solvent resulting from (c).

In some embodiments of the foregoing aspect, the method includes repeating steps (a)-(f) a plurality of times, optionally wherein the method includes repeating steps (a)-(f) continuously or discontinuously. In some embodiments, the method further includes adding fresh water-immiscible solvent to the cell culture composition following the selecting step.

In some embodiments, the plurality of the host cells is separated from the water-immiscible solvent by way of a gravity separation process. In some embodiments, the plurality of the host cells is further separated from the water-immiscible solvent using a centrifuge, hydrocyclone, or membrane. In some embodiments, the gravity separation process includes cell sedimentation. In some embodiments, the cell sedimentation is achieved using a gravity settling device. In some embodiments, the gravity settling device includes: (i) an inlet tube that is in fluid communication with, and that receives the portion of the mixture from, the vessel; (ii) a settling chamber that is in fluid communication with, and that receives the portion of the mixture from, the inlet tube, wherein the settling chamber has an incline angle of greater than 0° and less than, or equal to, 90°, optionally wherein the settling chamber has an incline angle of from about 25° to about 75°, optionally wherein the settling chamber has an incline angle of from about 35° to about 55°, optionally wherein the settling chamber has an incline angle of about 45°; (iii) an outlet at the bottom of the settling chamber that is in fluid communication with the vessel; (iv) an outlet at the top of the settling chamber that is in fluid communication with an effluent vessel; and, optionally, (v) an overflow outlet at the top of the inlet tube that is in fluid communication with the effluent vessel, whereby upon introduction into the inlet tube of an excess of the mixture that exceeds the volume of the settling chamber, the excess mixture flows through the overflow outlet and into the effluent vessel. In some embodiments, the cell sedimentation includes: (i) introducing the portion of the mixture into the inlet tube; (ii) allowing the plurality of the host cells to flow to the bottom of the settling chamber and, subsequently, to return to the vessel through the outlet at the bottom of the settling chamber; (iii) removing the water-immiscible solvent from the settling chamber through the outlet at the top of the settling chamber and delivering the water-immiscible solvent to the effluent bottle; and, optionally, (iv) removing any excess mixture that exceeds the volume of the settling chamber through the overflow outlet and delivering the excess mixture to the effluent vessel. In some embodiments, the gravity settling device further includes a bubble trap. In some embodiments, the bubble trap and the settling chamber are joined at an angle of between about 60° and 120° (e.g., an angle of about 60°, 65°, 70°, 75°, 80°, 85°, 90°, 95°, 100°, 105°, 110°, 115°, or 120°). In some embodiments, the bubble trap and the settling chamber are joined at an angle of about 45°.

In some embodiments, the portion of the mixture is delivered to the inlet tube by way of a pump. In some embodiments, the water-immiscible solvent is removed from the settling chamber and delivered through the outlet at the top of the settling chamber to the effluent bottle by way of a pump. In some embodiments, the method includes introducing a carbon source into the vessel. In some embodiments, the carbon source is continuously introduced into the vessel. In some embodiments, the carbon source is introduced into the vessel by way of a pump. In some embodiments, the method includes oxygenating the cell culture composition. In some embodiments, the cell culture composition is oxygenated by way of delivering compressed air into the vessel. In some embodiments, the method includes mixing the cell culture composition by way of an impeller.

In some embodiments, the cell culture product is a water-immiscible compound. In some embodiments, the cell culture product is a compound that is inhibitory to the host cells. In some embodiments, the cell culture product is a terpene. In some embodiments, the terpene is a C₅-C₄₀ terpene (e.g., a C₅ terpene, C₁₀ terpene, C₁₅ terpene, C₂₀ terpene, C₂₅ terpene, C₃₀ terpene, C₃₅ terpene, or C₄₀ terpene). In some embodiments, the terpene is a C₅-C₂₀ terpene (e.g., C₅ terpene, C₆ terpene, C₇ terpene, C₈ terpene, C₉ terpene, C₁₀ terpene, C₁₁ terpene, C₁₂ terpene, C₁₃ terpene, C₁₄ terpene, C₁₅ terpene, C₁₆ terpene, C₁₇ terpene, C₁₈ terpene, C₁₉ terpene, or C₂₀ terpene). In some embodiments, the terpene is a C₁₀-C₁₅ terpene (e.g., C₁₀ terpene, C terpene, C₁₂ terpene, C₁₃ terpene, C₁₄ terpene, or C₁₅ terpene). In some embodiments, the terpene is a hemiterpene, monoterpene, sesquiterpene, diterpene, sesterterpene, triterpene, tetraterpene, or polyterpene. In some embodiments, the terpene is a monoterpene.

In some embodiments, the cell culture product is an isoprenoid. In some embodiments, the isoprenoid is a C₅-C₄₀ isoprenoid (e.g., a C₅ isoprenoid, C₁₀ isoprenoid, C₁₅ isoprenoid, C₂₀ isoprenoid, C₂₅ isoprenoid, C₃₀ isoprenoid, C₃₅ isoprenoid, or C₄₀ isoprenoid). In some embodiments, the isoprenoid is a C₅-C₂₀ isoprenoid (e.g., C₅ isoprenoid, C₆ isoprenoid, C₇ isoprenoid, C₈ isoprenoid, C₉ isoprenoid, C₁₀ isoprenoid, C₁₁ isoprenoid, C₁₂ isoprenoid, C₁₃ isoprenoid, C₁₄ isoprenoid, C₁₅ isoprenoid, C₁₆ isoprenoid, C₁₇ isoprenoid, C₁₈ isoprenoid, C₁₉ isoprenoid, or C₂₀ isoprenoid). In some embodiments, the isoprenoid is a C₁₀-C₁₅ isoprenoid (e.g., C₁₀ isoprenoid, C₁₁ isoprenoid, C₁₂ isoprenoid, C₁₃ isoprenoid, C₁₄ isoprenoid, or C₁₅ isoprenoid). “In other embodiments, the isoprenoid is a C15 isoprenoid. These compounds are derived from three isoprene units and are also called sesquiterpenoids. Illustrative examples of sesquiterpenoids are periplanone B, gingkolide B, amorphadiene, artemisinin, artemisinic acid, valencene, nootkatone, epicedrol, epiaristolochene, farnesol, gossypol, sanonin, periplanone, forskolin, and patchoulol, which is also known as patchouli alcohol. In some embodiments, the isoprenoid is a hemiterpenoid, monoterpenoid, sesquiterpenoid, diterpenoid, sesterterpenoid, triterpenoid, tetraterpenoid, or polyterpenoid. In some embodiments, the isoprenoid is a monoterpenoid. In some embodiments, the cell culture product is the cell culture product is abietadiene, anethole, amorphadiene, carene, carvacrol, creosol, cuminaldehyde, eugenol, α-farnesene, β-farnesene, farnesol, geraniol, geranylgeraniol, hinokitiol, isoprene, linalool, limonene, myrcene, nerolidol, ocimene, patchoulol, β-pinene, sabinene, γ-terpinene, terpinolene, menthol, eucalyptol, citronellol, citronellal, valencene, or a salvinorin. In some embodiments, the cell culture product is β-farnesene. In some embodiments, the cell culture product is myrcene. In some embodiments, the cell culture product is pinene. In some embodiments, the cell culture product is limonene. In some embodiments, the cell culture product is hinokitiol. In some embodiments, the cell culture product is carvacrol. In some embodiments, the cell culture product is anethole. In some embodiments, the cell culture product is cuminaldehyde. In some embodiments, the cell culture product is a salvinorin. For example, the salvinorin may be salvinorin A, salvinorin B, salvinorin C, salvinorin D, salvinorin E, salvinorin F, salvinorin G, salvinorin H, salvinorin I, 17α-salvinorin J, or 17β-salvinorin J. In some embodiments, the cell culture product is eugenol. In some embodiments, the cell culture product is creosol.

In some embodiments, the cell culture product has a minimum inhibitory concentration (MIC) in the host cells of from about 0.1 mM to about 5 mM (e.g., about 0.1 mM to about 4 mM, about 0.1 mM to about 3 mM, about 0.1 mM to about 2 mM, about 0.1 mM to about 1 mM, about 0.1 mM to about 0.5 mM, about 0.5 mM to about 5 mM, about 1 mM to about 5 mM, about 2 mM to about 5 mM, about 3 mM to about 5 mM, or about 4 mM to about 5 mM). In some embodiments, the cell culture product has a MIC in the host cells of from about 0.1 mM to about 2.5 mM, optionally wherein the cell culture product has a MIC in the host cells of about 0.1 mM, about 0.15 mM, about 0.2 mM, about 0.25 mM, about 0.3 mM, about 0.35 mM, about 0.4 mM, about 0.45 mM, about 0.5 mM, about 0.55 mM, about 0.6 mM, about 0.65 mM, about 0.7 mM, about 0.75 mM, about 0.8 mM, about 0.85 mM, about 0.9 mM, about 0.95 mM, about 1 mM, about 1.25 mM, about 1.3 mM, about 1.35 mM, about 1.4 mM, about 1.45 mM, about 1.5 mM, about 1.55 mM, about 1.6 mM, about 1.65 mM, about 1.7 mM, about 1.75 mM, about 1.8 mM, about 1.85 mM, about 1.9 mM, about 1.95 mM, about 2 mM, about 2.25 mM, about 2.3 mM, about 2.35 mM, about 2.4 mM, about 2.45 mM, or about 2.5 mM.

In some embodiments, the water-immiscible solvent has a log(K_{d}) value of from about 1 to about 15, wherein the K_{d} is the partition coefficient for the cell culture product between the water-immiscible solvent and the cell culture composition. In some embodiments, the water-immiscible solvent has a log(K_{d}) value of from about 2 to about 3, optionally wherein the water-immiscible solvent has a log(K_{d}) value of about 2, about 2.1, about 2.2, about 2.3, about 2.4, about 2.5, about 2.6, about 2.7, about 2.8, about 2.9, or about 3. In some embodiments, the cell culture product has a log(D) value of from about 1 to about 15 (e.g., about 1.1, 1.2, 1.3, 1.4, 1.5, 1.6, 1.7, 1.8, 1.9, 2, 2.1, 2.2, 2.3, 2.4, 2.5, 2.6, 2.7 2.8, 2.9, 3.1, 3.2, 3.3, 3.4, 3.5, 3.6, 3.7, 3.8, 3.9, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.22, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, or 15).

In some embodiments, the water-immiscible solvent is an alcohol. In some embodiments, the water-immiscible solvent is a C₁₀-C₂₀ alcohol (e.g., C₁₀ alcohol, C₁₁ alcohol, C₁₂ alcohol, C₁₃ alcohol, C₁₄ alcohol, C₁₅ alcohol, C₁₆ alcohol, C₁₇ alcohol, C₁₈ alcohol, C₁₉ alcohol, or C₂₀ alcohol). In some embodiments, the water-immiscible solvent is a C₁₂-C₁₈ alcohol (e.g., C₁₂ alcohol, C₁₃ alcohol, C₁₄ alcohol, C₁₅ alcohol, C₁₆ alcohol, C₁₇ alcohol, or C₁₈ alcohol. In some embodiments, the water-immiscible solvent is a vegetable oil. In some embodiments, the water-immiscible solvent is corn oil, sunflower oil, soybean oil, mineral oil, polyalphaolefin, dodecane, hexadecane, oleyl alcohol, butyl oleate, dibutyl phthalate, dodecanol, dioctyl phthalate, farnesene, or isopropyl myristate.

In some embodiments, the cell culture product is a fermentation product. In some embodiments, the cell culture composition is a fermentation composition. In some embodiments, the vessel is a fermentation vessel.

### Definitions

As used in this specification and the appended claims, the singular forms “a,” “an,” and “the” include plural reference unless the context clearly dictates otherwise.

The term “about” when modifying a numerical value or range herein includes normal variation encountered in the field, and includes plus or minus 1-10% (e.g., 1%, 2%, 3%, 4%, 5%, 6%, 7%, 8%, 9% or 10%) of the numerical value or end points of the numerical range. Thus, a value of 10 includes all numerical values from 9 to 11. All numerical ranges described herein include the endpoints of the range unless otherwise noted, and all numerical values in-between the end points, to the first significant digit.

As used herein, the term “aqueous-phase culture medium” refers to a medium that is suitable for culturing host cells which uses water as the water-based medium.

As used herein in the context of extracting a cell culture product (e.g., a hydrophobic, lipophilic, and/or nonpolar cell culture product, such as a hydrophobic, lipophilic, and/or nonpolar fermentation product) present within a cell culture composition into an added solvent (e.g., a water-immiscible solvent described herein), the cell culture product is considered to be “partitioned between” the cell culture composition and the added solvent when the cell culture product reaches a distribution among the cell culture composition component and the added solvent component. The cell culture product may, for example, distribute such that the majority of the cell culture product is found within the added solvent (e.g., as in the case of a hydrophobic, lipophilic, or nonpolar cell culture product and a water-immiscible solvent). In some embodiments, the cell culture product distributes such that essentially all of the cell culture product is found within the added solvent. The cell culture product may be allowed, for example, to establish a dynamic equilibrium between the cell culture composition and the added solvent, such that the equilibrium concentration of the cell culture product within each component is reflective of the partition coefficient for the cell culture product and the mixture formed by the cell culture composition and added solvent. In some embodiments, the cell culture product is continuously extracted by the added solvent (e.g., through the use of fresh, added solvent), such that the majority (or essentially all) of the cell culture product is consistently found within the added solvent. In alternative embodiments, the cell culture product is discontinuously extracted by the added solvent (e.g., in a series of discrete extraction steps).

As used herein, the term “capable of producing” refers to a host cell which is genetically modified to include the enzymes necessary for the production of a given compound in accordance with a biochemical pathway that produces the compound. For example, a cell (e.g., a yeast cell) “capable of producing” an isoprenoid is one that contains the enzymes necessary for production of the isoprenoid according to the isoprenoid biosynthetic pathway.

As used herein, the term “cell sedimentation” refers to a process of separating a portion of cells from one or more other components of a composition over time wherein cells migrate toward a bottom surface of a vessel, e.g., by gravity. In some embodiments of the disclosure, cell sedimentation can be used to collect cells, e.g., from the cell culture composition, and recycle cells back to the cell culture mixture in order to maintain biomass. Therefore, cell sedimentation does not require cells to permanently settle at a bottom surface of a vessel.

As used herein, the term “cell culture product” refers to a compound that is produced by a host cell (e.g., yeast cell, bacteria cell, fungi cell, plant cell, or animal cell), which is cultured in a medium and under conditions suitable for the host cells to produce the cell culture product. The cell culture product may be naturally produced by the host cells or may be produced by host cells that have been genetically modified to produce the cell culture product. An example of a cell culture product is an isoprenoid or a terpene. In some embodiments, the cell culture product may be a fermentation product which refers to a cell culture product that is produced through a fermentation process, wherein the host cell is, for example, a yeast cell. In some embodiment, the cell culture product may be produced by a host cell that is a bacteria cell. Examples of host cells that may be used to produce a cell culture product are described below in the section titled Host Cell Strains.

As used herein, the term “cell culture composition” refers to a composition which contains host cells (e.g., yeast cells, bacteria cells, fungi cells, plant cells, or animal cells), and products or metabolites produced by the host cells, which may be genetically modified. An example of a cell culture composition is a whole cell broth, which may be the entire contents of a vessel, including cells, aqueous-phase culture medium, and compounds produced from the genetically modified host cells. In some embodiments, a cell culture composition of the disclosure may be contacted with a water-immiscible solvent. In some embodiments, the cell culture composition is a fermentation composition, wherein the fermentation composition contains yeast cells and products or metabolites produced by the yeast cells, which may be genetically modified. Examples of host cells that may be part of cell culture composition are described below in the section titled Host Cell Strains.

As used herein, the term “exogenous” refers a substance or compound that originated outside an organism or cell. The exogenous substance or compound can retain its normal function or activity when introduced into an organism or host cell described herein.

As used herein in the context of a gene, the term “express” refers to any one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. Expression of a gene of interest in a cell, tissue sample, or subject can manifest, for example, as: an increase in the quantity or concentration of mRNA encoding a corresponding protein (as assessed, e.g., using RNA detection procedures described herein or known in the art, such as quantitative polymerase chain reaction (qPCR) and RNA seq techniques), an increase in the quantity or concentration of a corresponding protein (as assessed, e.g., using protein detection methods described herein or known in the art, such as enzyme-linked immunosorbent assays (ELISA), among others), and/or an increase in the activity of a corresponding protein (e.g., in the case of an enzyme, as assessed using an enzymatic activity assay described herein or known in the art).

The term “expression cassette” or “expression construct” refers to a nucleic acid construct that, when introduced into a host cell, results in transcription and/or translation of an RNA or polypeptide, respectively. In the case of expression of transgenes, one of skill will recognize that the inserted polynucleotide sequence need not be identical but may be only substantially identical to a sequence of the gene from which it was derived. As explained herein, these substantially identical variants are specifically covered by reference to a specific nucleic acid sequence. One example of an expression cassette is a polynucleotide construct that includes a polynucleotide sequence encoding a polypeptide for use in the invention operably linked to a promoter, e.g., its native promoter, where the expression cassette is introduced into a heterologous microorganism. In some embodiments, an expression cassette includes a polynucleotide sequence encoding a polypeptide of the invention where the polynucleotide that is targeted to a position in the genome of a microorganism such that expression of the polynucleotide sequence is driven by a promoter that is present in the microorganism.

As used herein, the term “fermentation composition” refers to a cell culture composition which contains fermented host cells, for example yeast cells, and products or metabolites produced by the fermented host cells, which may be genetically modified. An example of a fermentation composition is a whole cell broth, which may be the entire contents of a vessel, including fermented host cells, aqueous-phase culture medium, and compounds produced from the genetically modified host cells. In some embodiments, a fermentation composition of the disclosure may be contacted with a water-immiscible solvent.

As used herein, the term “fermentation product” refers to a cell culture product that is produced through a fermentation process, wherein the host cell is, for example, a yeast cell. The fermentation product may be naturally produced by the host cells or may be produced by host cells that have been genetically modified to produce the fermentation product. An example of a fermentation product is an isoprenoid or terpene.

As used herein, the term “gene” refers to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, gRNA, or micro RNA.

A “genetic pathway” or “biosynthetic pathway” as used herein refer to a set of at least one coding sequence, where the coding sequence encodes an enzyme that catalyzes different parts of a synthetic pathway to form a desired product (e.g., cell culture product, e.g., an isoprenoid or a terpene, among others). In a genetic pathway a first encoded enzyme uses a substrate to make a first product which in turn is used as a substrate for a second encoded enzyme to make a second product. In some embodiments, the genetic pathway includes 2 or more members (e.g., 2, 3, 4, 5, 6, 7, 8, 9, etc.), wherein the product of one encoded enzyme is the substrate for the next enzyme in the synthetic pathway.

As used herein, the term “genetic switch” refers to one or more genetic elements that allow controlled expression of enzymes, e.g., enzymes that catalyze the reactions of a cell culture product (e.g., an isoprenoid or a terpene, among others) biosynthesis pathways. For example, a genetic switch can include one or more promoters operably linked to one or more genes encoding a biosynthetic enzyme, or one or more promoters operably linked to a transcriptional regulator which regulates expression one or more biosynthetic enzymes.

As used herein, the term “genetically modified” denotes a host cell that contains a heterologous nucleotide sequence. The genetically modified host cells described herein typically do not exist in nature.

As used herein, the term “gravity separation” refers to a process of separating two or more compositions from one another using gravity, optionally in addition to using gravity in the context of a gravity enhancing means, such as a centrifuge, hydrocyclone, or membrane.

As used herein, the term “heterologous” refers to what is not normally found in nature. The term “heterologous compound” refers to the production of a compound by a cell that does not normally produce the compound, or to the production of a compound at a level not normally produced by the cell. For example, a cell culture product (e.g., an isoprenoid or a terpene, among others) can be a heterologous compound.

A “heterologous genetic pathway” or a “heterologous biosynthetic pathway” as used herein refer to a genetic pathway that does not normally or naturally exist in an organism or cell.

The term “host cell” as used in the context of this invention refers to a microorganism, such as yeast, and includes an individual cell or cell culture contains a heterologous vector or heterologous polynucleotide as described herein. Host cells include progeny of a single host cell, and the progeny may not necessarily be completely identical (in morphology or in total DNA complement) to the original parent cell due to natural, accidental, or deliberate mutation and/or change. A host cell includes cells into which a recombinant vector or a heterologous polynucleotide of the invention has been introduced, including by transformation, transfection, and the like.

As used herein, the term “inhibitory” or “inhibitory effect” refers to negatively impacting cell health, including, but not limited to, growth, maintenance, and replication, or productivity.

As used herein, the term “medium” refers to culture medium and/or cell culture medium.

As used herein, the term “mixture” means a composition made of two or more different substances that are mixed. In some cases, a mixture described herein can be a homogenous mixture of the two or more different substances, e.g., the mixture can have the same proportions of its components (e.g., the two or more substances) throughout any given sample of the mixture. In some cases, a mixture as provided herein can be a heterogeneous mixture of the two or more different substances, e.g., the proportions of the components of the mixture (e.g., the two or more substances) can vary throughout the mixture. In some cases, a mixture is a liquid solution, e.g., the mixture is present in liquid phase. In some instances, a liquid solution can be regarded as comprising a liquid solvent and a solute. Mixing a solute in a liquid solvent can be termed as “dissolution” process. In some cases, there is more than one solvent and/or more than one solute. In some cases, a mixture is a colloid, liquid suspension, emulsion, or multiphasic.

The terms “modified,” “recombinant” and “engineered,” when used to modify a host cell described herein, refer to host cells or organisms that do not exist in nature, or express compounds, nucleic acids or proteins at levels that are not expressed by naturally occurring cells or organisms.

As used herein, the terms “oil,” “overlay oil,” or “overlay” refer to a biologically compatible hydrophobic, lipophilic, substance including but not limited to geologically-derived crude oil, distillate fractions of geologically-derived crude oil, vegetable oil, algal oil, microbial lipids, mineral oil, synthetic oils, or a derivative thereof. The oil is neither itself inhibitory to a biological molecule, a cell, a tissue, or a subject, nor does it degrade (if the oil degrades) at a rate that produces byproducts at inhibitory concentrations to a biological molecule, a cell, a tissue, or a subject. Preferred examples of oils include but are not limited to avocado oil, canola oil, grapeseed oil, hemp oil, soybean oil, jojoba oil, mineral oil, and sunflower oil.

As used herein, the phrase “operably linked” refers to a functional linkage between nucleic acid sequences such that the linked promoter and/or regulatory region functionally controls expression of the coding sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid.

The term “performance enhancement” refers to relative cell culture performance increase as defined as the ratio of the cell culture performance of the culture implementing the claimed methods to the cell culture performance of the culture without the claimed methods. Cell culture performance parameters could include cell culture yield, cell culture productivity or cell culture length. The range of performance enhancement could be from 1×-10× the cell culture performance of the culture without the claimed methods.

The terms “polynucleotide” and “nucleic acid” are used interchangeably and refer to a single or double-stranded polymer of deoxyribonucleotide or ribonucleotide bases read from the 5′ to the 3′ end. A nucleic acid as used in the present invention will generally contain phosphodiester bonds, although in some cases, nucleic acid analogs may be used that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); positive backbones; non-ionic backbones, and non-ribose backbones. Nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus, the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA, or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. Nucleic acid sequences are presented in the 5′ to 3′ direction unless otherwise specified.

As used herein, the terms “polypeptide,” “peptide,” and “protein” are used interchangeably to refer to a polymer of amino acid residues. The terms encompass amino acid chains of any length, including full-length proteins, wherein the amino acid residues are linked by covalent peptide bonds.

As used herein, the term “production” generally refers to an amount of compound produced by a genetically modified host cell provided herein. In some embodiments, production is expressed as a yield of the compound by the host cell. In other embodiments, production is expressed as a productivity of the host cell in producing the compound.

As used herein, the term “productivity” refers to production of a compound by a host cell, expressed as the amount of non-catabolic compound produced (by weight) per amount of cell culture broth in which the host cell is cultured (by volume) over time (per hour).

As used herein, the term “promoter” refers to a synthetic or naturally derived nucleic acid that is capable of activating, increasing or enhancing expression of a DNA coding sequence, or inactivating, decreasing, or inhibiting expression of a DNA coding sequence. A promoter may contain one or more specific transcriptional regulatory sequences to further enhance or repress expression and/or to alter the spatial expression and/or temporal expression of the coding sequence. A promote′ may be positioned 5′ (upstream) of the coding sequence under its control. A promoter may also initiate transcription in the downstream (3′) direction, the upstream (5′) direction, or be designed to initiate transcription in both the downstream (3′) and upstream (5′) directions. The distance between the promoter and a coding sequence to be expressed may be approximately the same as the distance between that promoter and the native nucleic acid sequence it controls. As is known in the art, variation in this distance may be accommodated without loss of promoter function. The term also includes a regulated promoter, which generally allows transcription of the nucleic acid sequence while in a permissive environment (e.g., microaerobic fermentation conditions, or the presence of maltose), but ceases transcription of the nucleic acid sequence while in a non-permissive environment (e.g., aerobic fermentation conditions, or in the absence of maltose). Promoters used herein can be constitutive, inducible, or repressible.

As used herein, the term “water-immiscible solvent” refers to a solvent which does not form a homogenous mixture when added to water. A water-immiscible solvent may be an alcohol (e.g., a C₁₀-C₂₀ alcohol). In some embodiments, the water-immiscible solvent is an oil. The water-immiscible solvent may also be, for example, corn oil, sunflower oil, soybean oil, mineral oil, polyalphaolefin, dodecane, hexadecane, oleyl alcohol, butyl oleate, dibutyl phthalate, dodecanol, dioctyl phthalate, farnesene, or isopropyl myristate. In some embodiments, the water-immiscible solvent is a Drakeol™ fluid. In some embodiments, the water-immiscible solvent is a Jarcol™ fluid. In some embodiments, the water-immiscible solvent is a Durasyn™ fluid.

The term “yield” refers to production of a compound by a host cell, expressed as the amount of compound produced per amount of carbon source consumed by the host cell, by weight.

### BRIEF DESCRIPTION OF THE DRAWINGS

The application file contains at least one drawing executed in color. Copies of this patent or patent application with color drawings will be provided by the Office upon request and payment of the necessary fee.

FIG. 1 is a schematic drawing showing an exemplary angle of the cell settling zone of a gravity settling device.

FIG. 2 is a schematic drawing showing the general setup of a gravity set-ling device.

FIG. 3A-FIG. 3C are photographs of a gravity settling device setup, including a settling zone, effluent outlet, bubble trap, bubble trap vent, and inlet (FIG. 3A) as well as the pinch valves (FIG. 3B) and timers (FIG. 3C).

FIG. 4A and FIG. 4B are graphs showing the percentage of packed cell volume (PCV) remaining over time (FIG. 4A), and the biomass mass retention in the whole cell broth in comparison to the effluent stream (FIG. 4B) of a cell culture composition cultured using the Y21900 yeast strain to produce a farnesene product using a commercially available gravity settling device.

FIG. 5A and FIG. 5B are graphs showing the concentration of farnesene in the whole cell broth over time (FIG. 5A) and cell retention over time in the whole cell broth and the effluent stream (FIG. 5B) of a cell culture composition. For these experiments, the Y21900 yeast strain was cultured to produce a farnesene product using a commercially available gravity settling device. Productivity results were then compared to an −AD control.

FIG. 6A-FIG. 6D are graphs showing the percent yield (FIG. 6A), productivity (FIG. 6B), the oxygen uptake (FIG. 6C), and the carbon balance (FIG. 6D) of a cell culture composition cultured using the Y21900 yeast strain to produce a farnesene product when a commercially available gravity settling device was used as compared to a FAD control.

FIG. 7 is a graph showing the predicted recovery yield over time for when the commercially available gravity settler was used to remove the farnesene product produced in a cell culture composition using the Y21900 yeast strain as compared to a FAD control.

FIG. 8A-FIG. 8C are graphs showing the PCV (FIG. 8A), cell retention (FIG. 8B), average oxygen uptake (FIG. 8C) of a cell culture composition cultured using the Y23508 yeast strain to produce a farnesene product when an in-house gravity settling device was used as compared to a FAD control.

FIG. 9A and FIG. 9B are graphs showing the concentration of farnesene in the whole cell broth over time (FIG. 9A) and cell retention over time in the whole cell broth and the effluent stream (FIG. 9B) of a cell culture composition cultured using the Y23508 yeast strain to produce a farnesene product when an in-house available gravity settling device was used in comparison to a FAD control.

FIG. 10A-FIG. 10C are graphs showing the percent yield (FIG. 10A), productivity (FIG. 10B), the oxygen uptake (FIG. 10C), and the carbon balance (FIG. 6D) of a cell culture composition cultured using the Y23508 yeast strain to produce a farnesene product when an in-house gravity settling device was used in comparison to a FAD control.

FIG. 11A-FIG. 11C are graphs showing the percent yield (FIG. 11A), biomass retention (FIG. 11B), and oxygen uptake (FIG. 11C), over time of cell culture compositions that were cultured using a dilute cane sugar stock and the Y23508 yeast strain to produce a farnesene product in comparison to dilute can sugar stock control and a FAD control.

FIG. 12A and FIG. 12B are graphs showing the concentration farnesene in the whole cell broth over time (FIG. 12A) and cell retention over time in the whole cell broth and the effluent stream (FIG. 12B) of cell culture compositions that were cultured using a dilute cane sugar stock and the Y23508 yeast strain to produce a farnesene product in comparison to dilute can sugar stock control and a FAD control.

FIG. 13A-FIG. 13D are graphs showing the farnesene yield (FIG. 13A), productivity (FIG. 13B), a 24-hour interval fermentation yield (FIG. 13C), and a 24-hour interval productivity (FIG. 13D) over time of cell culture compositions that were cultured using a dilute cane sugar stock and the Y23508 yeast strain to produce a farnesene product in comparison to dilute can sugar stock control and a FAD control.

FIG. 14A-FIG. 14B are graphs showing the carbon balance (FIG. 14A) and the mass balance (FIG. 14B) of over time of cell culture compositions that were cultured using a dilute cane sugar stock and the Y23508 yeast strain to produce a farnesene product in comparison to dilute can sugar stock control and a FAD control.

FIG. 15A-FIG. 15B are graphs showing the amount of dissolved oxygen (pO₂) present in the cell culture composition, the feed rate, and the oxygen uptake rate (OUR) over a period of 160 hours with 25% of full volume overlay (FIG. 15A) or 5% of full volume overlay (FIG. 151B).

### DETAILED DESCRIPTION

The present disclosure provides compositions and methods for producing a cell culture product (e.g., fermentation product) in a cell culture composition (e.g., fermentation composition), particularly for cell culture products that impair the growth and/or function of the host cells in which they are biosynthesized, when cell biomass is low, such as in the case of a poor growing strain, or when feed sugar concentrations are low. For example, the compounds produced by cell culture may be inhibitory to the host cells capable of producing the product, such that as the concentration of the product increases, inhibitory effect in the host cell increases in kind. This inhibitory effect makes production of certain types of compounds challenging. This is the case for many isoprenoids, and in particular, terpene compounds.

It has been presently discovered that continuous extraction of a cell culture product from a vessel and replacement of the cell culture composition following extraction of the cell culture product result is higher yields of the cell culture product and increased productivity of the host cells. For example, using the methods and compositions described herein, a terpene product, such as abietadiene, amorphadiene, carene, α-farnesene, β-farnesene, farnesol, geraniol, geranylgeraniol, isoprene, linalool, limonene, myrcene, nerolidol, ocimene, patchoulol, β-pinene, sabinene, γ-terpinene, terpinolene, menthol, eucalyptol, citronellol, citronellal, or valencene, may be isolated from a cell culture composition that is produced by culturing host cells capable of synthesizing a terpene in a culture medium and under conditions suitable for the host cells to produce a terpene. To isolate the terpene from the cell culture composition, the cell culture composition may be contacted with a water-immiscible solvent and the terpene may be partitioned between the water-immiscible solvent and the cell culture composition such that the terpene can be removed from the vessel. The resulting terpene may then be recovered from the water-immiscible solvent. The host cells from the cell culture composition may then be returned to the vessel for continued culture. Additionally, a gravity settling device may be used to separate the host cells from the cell culture product and recycle them back into the cell culture composition, which allows for increased retention of biomass and an increase in yield of the cell culture product.

The sections that follow provide a description of exemplary compositions and methods that may be used to culture host cells capable of producing a cell culture product, as well as compositions and methods for isolating the cell culture product from the cell culture composition.

### Cell Culture Product

Described herein are methods of producing a cell culture product (e.g., fermentation product), as well as methods of isolating a cell culture product from a cell culture composition (e.g., fermentation composition). In some embodiments, the cell culture product is a water-immiscible compound. For example, the cell culture product may have a log(D) value of from about 1 to about 15, or the water-immiscible may have a log(K_{d}) value of from about 1 to about 15, where K_{d} is the partition coefficient for the cell culture product between the water-immiscible solvent and the cell culture composition.

In some embodiments, the cell culture product is a compound that is inhibitory to the host cells. As discussed above, some compounds of interest are inhibitory to the host cells such that as the host cells produce the cell culture product in the cell culture composition, accumulation of the cell culture product decreases host cell health. Examples of cell culture products include isoprenoids and terpenes, among others known in the art and described herein.

### Isoprenoids

In some embodiments, the cell culture product is an isoprenoid. The isoprenoid may be a C₅-C₄₀ isoprenoid (e.g., a C₅ isoprenoid, C₁₀ isoprenoid, C₁₅ isoprenoid, C₂₀ isoprenoid, C₂₅ isoprenoid, C₃₀ isoprenoid, C₃₅ isoprenoid, or C₄₀ isoprenoid). For example, in some embodiments, the isoprenoid is a C₂₀ isoprenoid. These compounds are derived from four isoprene units and also called diterpenoids. Illustrative examples of diterpenoids are casbene, eleutherobin, paclitaxel, prostratin, pseudopterosin, and taxadiene. In yet other examples, the isoprenoid is a C₂₀₊ isoprenoid. These compounds are derived from more than four isoprene units and include: triterpenoids (C₃₀ isoprenoid compounds derived from 6 isoprene units) such as arbrusidee, bruceantin, testosterone, progesterone, cortisone, digitoxin, and squalene; tetraterpenoids (C40 isoprenoid compounds derived from 8 isoprenoids) such as P-carotene; and polyterpenoids (C₄₀₊ isoprenoid compounds derived from more than 8 isoprene units) such as polyisoprene. In other embodiments, the isoprenoid is a C15 isoprenoid. These compounds are derived from three isoprene units and are also called sesquiterpenoids. Illustrative examples of sesquiterpenoids are periplanone B, gingkolide B, amorphadiene, artemisinin, artemisinic acid, valencene, nootkatone, epicedrol, epiaristolochene, farnesol, gossypol, sanonin, periplanone, forskolin, and patchoulol, which is also known as patchouli alcohol. In some embodiments, the isoprenoid is selected from the group consisting of abietadiene, amorphadiene, carene, a-farnesene, P-farnesene, farnesol, geraniol, geranylgeraniol, isoprene, linalool, limonene, myrcene, nerolidol, ocimene, patchoulol, P-pinene, sabinene, y-terpinene, terpinolene, valencene, retinol, phytol, retinal, santalol, santalene, sinensol, squalene, bisabolol, and sclareol.

The isoprenoid cell culture product may be a C₅-C₂₀ isoprenoid (e.g., C₅ isoprenoid, C₆ isoprenoid, C₇ isoprenoid, C₈ isoprenoid, C₉ isoprenoid, C₁₀ isoprenoid, C₁₁ isoprenoid, C₁₂ isoprenoid, C₁₃ isoprenoid, C₁₄ isoprenoid, C₁₅ isoprenoid, C₁₆ isoprenoid, C₁₇ isoprenoid, C₁₈ isoprenoid, C₁₉ isoprenoid, or C₂₀ isoprenoid). In some embodiments, the isoprenoid produced by the cell is a C₅ isoprenoid. These compounds are derived from one isoprene unit and are also called hemiterpenoids. An illustrative example of a hemiterpenoid is isoprene.

The isoprenoid cell culture product may be a C₁₀-C₁₅ isoprenoid (e.g., C₁₀ isoprenoid, C₁₁ isoprenoid, C₁₂ isoprenoid, C₁₃ isoprenoid, C₁₄ isoprenoid, or C₁₅ isoprenoid). In other embodiments, the isoprenoid is a C₁₀ isoprenoid. These compounds are derived from two isoprene units and are also called monoterpenoids. Illustrative examples of monoterpenoids are limonene, citranellol, geraniol, menthol, perillyl alcohol, linalool, thujone, and myrcene. In other embodiments, the isoprenoid is a C₁₅ isoprenoid. These compounds are derived from three isoprene units and are also called sesquiterpenoids. Illustrative examples of sesquiterpenoids are periplanone B, gingkolide B, amorphadiene, artemisinin, artemisinic acid, valencene, nootkatone, epicedrol, epiaristolochene, farnesol, gossypol, sanonin, periplanone, forskolin, and patchoulol, which is also known as patchouli alcohol.

Isoprenoid compounds also include, but are not limited to, carotenoids (such as lycopene, a- and P-carotene, a- and P-cryptoxanthin, bixin, zeaxanthin, astaxanthin, and lutein), steroid compounds, cannabinoids, and compounds that are composed of isoprenoids modified by other chemical groups, such as mixed terpene-alkaloids, and coenzyme Q-10.

In some embodiments, the isoprenoid is a hemiterpenoid, monoterpenoid, sesquiterpenoid, diterpenoid, sesterterpenoid, triterpenoid, tetraterpenoid, or polyterpenoid. In some embodiments, the isoprenoid is a monoterpenoid.

### Terpenes

In some embodiments, the cell culture product is a terpene. In some embodiments, the terpene is a C₅-C₄₀ terpene (e.g., a C₅ terpene, C₁₀ terpene, C₁₅ terpene, C₂₀ terpene, C₂₅ terpene, C₃₀ terpene, C₃₅ terpene, or C₄₀ terpene). In some embodiments, the terpene is a C₅-C₂₀ terpene (e.g., C₅ terpene, C₆ terpene, C₇ terpene, C₈ terpene, C₉ terpene, C₁₀ terpene, C₁₁ terpene, C₁₂ terpene, C₁₃ terpene, C₁₄ terpene, C₁₅ terpene, C₁₆ terpene, C₁₇ terpene, C₁₈ terpene, C₁₉ terpene, or C₂₀ terpene). In some embodiments, the terpene is a C₁₀-C₁₅ terpene (e.g., C₁₀ terpene, C₁₁ terpene, C₁₂ terpene, C₁₃ terpene, C₁₄ terpene, or C₁₅ terpene). In some embodiments, the terpene is a hemiterpene, monoterpene, sesquiterpene, diterpene, sesterterpene, triterpene, tetraterpene, or polyterpene.

In some embodiments, the terpene is a monoterpene. Monoterpenes are C₁₀ terpenes and are derived from two isoprene units. For example, the monoterpene may be carene, whose structure is:

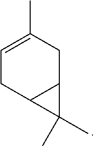

Carene is typically made from GPP by carene synthase. Illustrative examples of suitable nucleotide sequences include but are not limited to: (AF461460, REGION 43 . . . 1926: Picea abies) and (AF527416, REGION: 78 . . . 1871; Salvia stenophylla) for use as heterologous sequences that encode carene synthase. Another monoterpene, such as geraniol, (also known as rhodnol), whose structure is

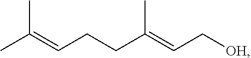

may be a product produced by the present invention. Geraniol is typically made from OPP by geraniol synthase. Illustrative examples of suitable nucleotide sequences include but are not limited to: (AJ457070; Cinnamomum tenuipilum), (AY362553; Ocimum basilicum), (DQ234300; Perilla frutescens strain 1864), (DQ234299; Perilla citriodora strain 1861), (DQ234298; Perilla citriodora strain 4935), and (DQ088667; Perilla citriodora) for encoding geraniol synthase that may be used a heterologous sequence of the present invention. The monoterpene, linalool, whose structure is:

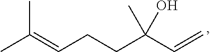

is typically made from GPP by linalool synthase and may be produced by the present invention. Illustrative examples of a suitable nucleotide sequence include, but are not limited to: (AF497485; Arabidopsis thaliana), (AC002294, Locus AAB71482; Arabidopsis thaliana), (AY059757; Arabidopsis thaliana), (NM-104793; Arabidopsis thaliana), (AF154124; Artemisia annua), (AF067603; Clarkia breweri), (AF067602; Clarkia concinna), (AF067601; Clarkia breweri), (U58314; Clarkia breweri), (AY840091; Lycopersicon esculentum), (DQ263741; Lavandula angustifolia), (AY083653; Mentha citrate), (AY693647; Ocimum basilicum), (XM-463918; Oryza sativa), (AP004078, Locus BAD07605; Oryza sativa), (XM-463918, Locus XP-463918; Oryza sativa), (AY917193; Perilla citriodora), (AF271259; Perilla frutescens), (AY473623; Picea abies), (DQ195274; Picea sitchensis), and (AF444798; Perilla frutescens var. crispa cultivar No. 79). These sequences may be used as heterologous sequences of the present invention. Another monoterpene, limonene whose structure is:

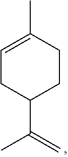

is typically made from GPP by limonene synthase. Illustrative examples of suitable nucleotide sequences that may be used as heterologous sequences of the present invention include but are not limited to: (+)-limonene synthases (AF514287, REGION: 47 . . . 1867; Citrus limon) and (AY055214, REGION: 48 . . . 1889; Agastache rugosa) and (−)-limonene synthases (DQ195275, REGION: 1 . . . 1905; Picea sitchensis), (AF006193, REGION: 73.1986; Abies grandis), and (MC4SLSP, REGION: 29 . . . 1828; Mentha spicata).

The monoterpene, myrcene, whose structure is:

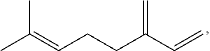

is typically made from GPP by myrcene synthase and is another product that may be produced by the present invention. Illustrative examples of suitable nucleotide sequences that may be used as heterologous sequences of the present invention include but are not limited to: (187908; Abies grandis), (AY195609; Antirrhinum majus), (AY195608; Antirrhinum majus), (NM-127982; Arabidopsis thaliana TPS10), NM-113485; Arabidopsis thaliana ATTPS-CIN), (NM-13483; Arabidopsis thaliana ATIPS-CIN), (AF271259; Perilla frutescens), (AY473626; Picea abies), (AF369919; Picea abies), and (AJ304839; Quercus ilex).

Another monoterpene, ocimene, α- and β-Ocimene, whose structures are:

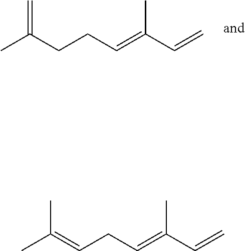

respectively, are typically made from GPP by ocimene synthase, a synthase that may be encoded by the heterologous sequences of the present invention. Illustrative examples of suitable nucleotide sequences that may be used as heterologous sequences include but are not limited to: (AY195607; Antirrhinum majus), (AY195609; Antirrhinum majus), (AY195608; Antirrhinum majus), (AK221024; Arabidopsis thaliana), (NM-113485; Arabidopsis thaliana ATTPS-CIN), (NM-113483; Arabidopsis thaliana ATTPS-CIN), (NM-117775; Arabidopsis thaliana ATTPS03), (NM-001036574; Arabidopsis thaliana ATTPS03), (NM-127982; Arabidopsis thaliana TPS10), (AB110642; Citrus unshiu CitMTSL4), and (AY575970; Lotus corniculatus var. japonicus).

Another monoterpene, α-pinene whose structure is:

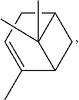

is typically made from GPP by α-pinene synthase, a synthase that may be encoded by the heterologous sequences of the present invention. Illustrative examples of suitable nucleotide sequences that may be used as heterologous sequences to encode the synthase include but are not limited to: (+) α-pinene synthase (AF543530, REGION: 1 . . . 1887; Pinus taeda), (−)α-pinene synthase (AF543527, REGION: 32 . . . 1921; Pinus taeda), and (+)/(−)α-pinene synthase (AGU87909, REGION: 6111892; Abies grandis).

Another monoterpene, β-pinene, whose structure is:

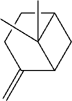

is typically made from GPP by β-pinene synthase, a synthase that may be encoded by the heterologous sequences of the present invention. Illustrative examples of suitable nucleotide sequences that may be used as heterologous sequences to encode the synthase include but are not limited to: (−) β-pinene synthases (AF276072, REGION: 1 . . . 1749; Artemisia annua) and (AF514288, REGION: 26 . . . 1834; Citrus limon).

Another monoterpene, sabinene, whose structure is:

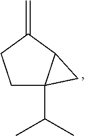

is typically made from GPP by sabinene synthase, a synthase that may be encoded by the heterologous sequences of the present invention. An illustrative example of a suitable nucleotide sequence that may be used as a heterologous sequence of include but is not limited to AF051901, REGION: 26 . . . 1798 from Salvia officinalis.

Another monoterpene, γ-terpinene, whose structure is:

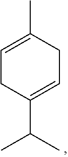

is typically made from GPP by a γ-terpinene synthase, a synthase that may be encoded by the heterologous sequences of the present invention. Illustrative examples of suitable nucleotide sequences that may be used as heterologous sequences include but are not limited to: (AF514286, REGION: 30 . . . 1832 from Citrus limon) and (AB110640, REGION 1 . . . 1803 from Citrus unshiu).

Another monoterpene, terpinolene, whose structure is

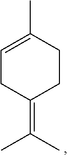

is typically made from GPP by terpinolene synthase, a synthase that may be encoded by the heterologous sequences of the present invention. Illustrative examples of suitable nucleotide sequences that may be used as heterologous sequences include but are not limited to: (AY693650 from Oscimum basilicum) and (AY906866, REGION: 10 . . . 1887 from Pseudotsuga menziesii).

In some embodiments, the cell culture product is a diterpene. In some embodiments, the diterpene is a salvinorin.

In some embodiments, the cell culture product is abietadiene, anethole, amorphadiene, carene, carvacrol, creosol, cuminaldehyde, eugenol, α-farnesene, β-farnesene, farnesol, geraniol, geranylgeraniol, hinokitiol, isoprene, linalool, limonene, myrcene, nerolidol, ocimene, patchoulol, β-pinene, sabinene, γ-terpinene, terpinolene, menthol, eucalyptol, citronellol, citronellal, valencene, or a salvinorin. In some embodiments, the cell culture product is β-farnesene. In some embodiments, the cell culture product is myrcene. In some embodiments, the cell culture product is pinene. In some embodiments, the cell culture product is limonene. In some embodiments, the cell culture product is hinokitiol. In some embodiments, the cell culture product is carvacrol. In some embodiments, the cell culture product is anethole. In some embodiments, the cell culture product is cuminaldehyde. In some embodiments, the cell culture product is a salvinorin. For example, the salvinorin may be salvinorin A, salvinorin B, salvinorin C, salvinorin D, salvinorin E, salvinorin F, salvinorin G, salvinorin H, salvinorin I, 17α-salvinorin J, or 17β-salvinorin J. In some embodiments, the cell culture product is eugenol. In some embodiments, the cell culture product is creosol.

### Methods of Producing a Cell Culture Product

Provided herein are methods for producing one or more cell culture products (e.g., fermentation products) from a cell culture composition (e.g., fermentation composition), where the cell culture product may be a water-immiscible compound. The methods described herein may include a number of steps to isolate the cell culture product produced by the host cells in the cell culture composition. These steps may include contacting the cell culture composition with a water-immiscible solvent to form a mixture and partition the cell culture product between the cell culture composition and the water-immiscible solvent such that the cell culture product can be removed from the cell culture composition, and the host cells can be recycled into the cell culture composition; or these steps may include isolating a water-immiscible cell culture product from a cell culture composition by isolating from the vessel a portion of the cell culture composition; separating the host cells from the portion of the isolated cell culture composition; returning the host cells to the vessel; and recovering the water-immiscible cell culture product from the isolated the cell culture composition.

While the processes and systems provided herein have been described with respect to a limited number of embodiments, the specific features of one embodiment should not be attributed to other embodiments of the processes or systems. No single embodiment is representative of all aspects of the methods or systems. In certain embodiments, the processes can include numerous steps not mentioned herein. In some embodiments, the processes do not include any steps not described herein. Variations and modifications from the described embodiments exist.

The cell culture product may be produced by culturing host cells capable of synthesizing the cell culture product in a vessel. The vessel may have a capacity of between 1,000,000 L and 0.5 L; for example, the vessel may have a capacity of between 0.5 L and 500,000 L, 0.5 L and 100,000 L, or 0.5 L and 1,000 L.

A carbon source may be introduced into the vessel for culturing the host cells. In some embodiments, the carbon source is a carbohydrate. In other embodiments, the carbon source is an alcohol (e.g., ethanol, glycerol, etc.). In other embodiments, the carbon source is obtained from second generation sugars (e.g., digestions of lignocellulose). In some embodiments, the carbon source is continuously introduced into the vessel. The carbon source may be introduced into the vessel by way of a pump. The carbon source may be introduced into the vessel at a rate of from about 0.01 g total reducing sugar (TRS)/L/hour to about 25 g TRS/L/hour. In some embodiments, the carbon source may be introduced into the vessel at a rate of from about 0.01 g TRS/L/hour to about 5 g TRS/L/hour. In some embodiments, the carbon source may be introduced into the vessel at a rate of from about 0.01 g TRS/L/hour to about 0.5 g TRS/L/hour. For example, the carbon source may be introduced into the vessel at a rate of less than 0.15 g TRS/L/hour, such as a rate of from about 0.01 g TRS/L/hour to about 0.15 g TRS/L/hour. The carbon source that is being introduced into the vessel may have a concentration of about 3% (w/v) to about 80% (w/v) of total reducing sugar; for example, it may have a concentration of about 10% (w/v) to about 70% (w/v), about 30% (w/v) of total reducing sugar, or it may have a concentration of about 60% (w/v) of total reducing sugar.

In some embodiments, culturing conditions are anaerobic. In other embodiments, culturing conditions are aerobic or microaerobic and the culture medium includes cells which consume oxygen. They may consume oxygen at a rate of from about 25 mmol/L/hr to about 300 mmol/L/hr (e.g., about 25 mmol/L/hr to about 225 mmol/L/hr, about 25 mmol/L/hr to about 200 mmol/L/hr, about 25 mmol/L/hr to about 175 mmol/L/hr, about 25 mmol/L/hr to about 150 mmol/L/hr, about 25 mmol/L/hr to about 125 mmol/L/hr, about 25 mmol/L/hr to about 100 mmol/L/hr, about 25 mmol/L/hr to about 75 mmol/L/hr, about 75 mmol/L/hr to about 250 mmol/L/hr, about 100 mmol/L/hr to about 250 mmol/L/hr, about 125 mmol/L/hr to about 250 mmol/L/hr, about 150 mmol/L/hr to about 250 mmol/L/hr, about 175 mmol/L/hr to about 250 mmol/L/hr, about 200 mmol/L/hr to about 250 mmol/L/hr, or about 225 mmol/L/hr to about 250 mmol/L/hr). In some embodiments, the host cells in the cell culture composition consume oxygen at a rate of from about 90 mmol/L/hr to about 130 mmol/L/hr (e.g., about 90 mmol/L/hr to about 120 mmol/L/hr, about 90 mmol/L/hr to about 110 mmol/L/hr, about 90 mmol/L/hr to about 100 mmol/L/hr, about 100 mmol/L/hr to about 130 mmol/L/hr, about 110 mmol/L/hr to about 130 mmol/L/hr, or about 120 mmol/L/hr to about 130 mmol/L/hr). For example, the host cells in the cell culture composition consume oxygen at a rate of from about 90 mmol/L/hr to about 130 mmol/L/hr. In some embodiments, the host cells in the cell culture composition consume oxygen at a rate of from about 110 mmol/L/hr.

### Aqueous Phase Separation

The aqueous phase containing the cells may be separated from the oil-emulsion phase containing the product using a gravity separation. The gravity separation process may include cell sedimentation. In some embodiments, the gravity separation is achieved using a gravity settling device.

In some embodiments, the gravity settling device may include an inlet tube that is in fluid communication with, and that receives the portion of the mixture from, the vessel. In some embodiments, the gravity settling device may include a settling chamber that is in fluid communication with, and that receives the portion of the mixture from, the inlet tube. The settling chamber may have an incline angle of greater than 0° and less than, or equal to, 90°. For example, the settling chamber may have an incline angle of from about 25° to about 75°. In some embodiments the settling chamber may have an incline angle of about 45°. In some embodiments, the gravity settling device may include an outlet at the bottom of the settling chamber that is in fluid communication with the vessel. In some embodiments, the gravity settling device includes an outlet at the top of the settling chamber that is in fluid communication with an effluent vessel. In some embodiments, the gravity settling device includes an overflow outlet at the top of the inlet tube that is in fluid communication with the effluent vessel. In some embodiments, the overflow may be returned to the effluent vessel.

In some embodiments, the cell sedimentation process may include introducing the portion of the mixture including the cell culture composition into the inlet tube. In some embodiments, the plurality of the host cells to flow to the bottom of the settling chamber. These host cells may then return to the vessel through the outlet at the bottom of the settling chamber. In some embodiments, the water-immiscible solvent is removed from the settling chamber through the outlet at the top of the settling chamber and the water-immiscible solvent is delivered to the effluent bottle. In some embodiments, any excess mixture that exceeds the volume of the settling chamber is removed through the overflow outlet and delivered the to the effluent vessel. The host cells may be sedimented at a rate of about 0.03 mm/min or greater. For example, the host cells are sedimented at a rate of from about 0.003 mm/min or greater, optionally wherein the host cells are sedimented at a rate of from about 0.003 mm/min to about 0.5 mm/min (e.g., about 0.003 mm/min to about 0.4 mm/min, about 0.003 mm/min to about 0.3 mm/min, about 0.003 mm/min to about 0.2 mm/min, about 0.003 mm/min to about 0.1 mm/min, about 0.003 mm/min to about 0.05 mm/min, about 0.003 mm/min to about 0.01 mm/min, about 0.003 mm/min to about 0.005 mm/min, about 0.005 mm/min to about 0.5 mm/min, about 0.01 mm/min to about 0.5 mm/min, about 0.05 mm/min to about 0.5 mm/min, about 0.1 mm/min to about 0.5 mm/min about 0.2 mm/min to about 0.5 mm/min, about 0.3 mm/min to about 0.5 mm/min, or about 0.4 mm/min to about 0.5 mm/min). In some embodiments, the plurality of the host cells is returned to the vessel at a rate of from about 1 ml/L/min to about 300 ml/L/min (e.g., about 1 ml/L/min to about 250 ml/L/min, about 1 ml/L/min to about 200 ml/L/min, about 1 ml/L/min to about 150 ml/L/min, about 1 ml/L/min to about 100 ml/L/min, about 1 ml/L/min to about 50 ml/L/min, about 1 ml/L/min to about 25 ml/L/min, about 2 ml/L/min to about 300 ml/L/min, about 3 ml/L/min to about 300 ml/L/min, about 4 ml/L/min to about 300 ml/L/min, about 10 mL/L/min to about 300 mL/L/min, about 50 mL/L/min to about 300 mL/L/min, about 100 mL/L/min to about 300 mL/L/min, about 150 mL/L/min to about 300 mL/L/min, about 200 mL/L/min to about 300 mL/L/min, or about 250 mL/L/min to about 300 mL/L/min).

A pump may be used in the gravity settling device. In some embodiments, the portion of the mixture including the cell culture composition is delivered to the inlet tube by way of a pump. In some embodiments, the water-immiscible solvent is removed from the settling chamber and delivered through the outlet at the top of the settling chamber to the effluent bottle by way of a pump.

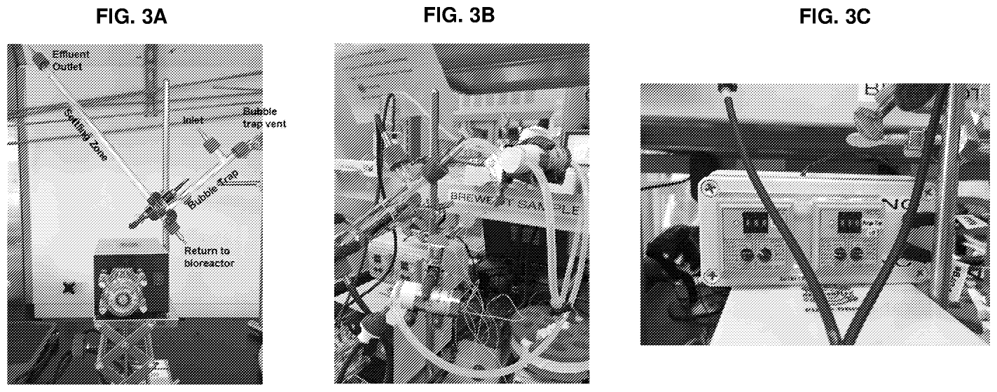

Pinch valves may be used in to control the amount of cell culture composition entering the cell settling device. These pinch valves may be controlled by timers, e.g., as shown in FIG. 3B and FIG. 3C. In some embodiments, the opening and closing of pinch valves may be automated by monitoring the volume of liquid in the bubble trap. The volume of liquid in the bubble trap may be monitored, for example, using a float switch, a light curtain sensor, or sensors for monitoring capacitance.

The aqueous phase of the cell culture composition may be oxygenated. Oxygenating of the aqueous phase of the cell culture composition may be by pumping air into the vessel. The air may be compressed air. The compressed air may be delivered by pressure.

The aqueous phase of the cell culture composition may be mixed using an impeller. Furthermore, the aqueous phase of the cell culture composition and the water-immiscible solvent may be mixed using an impeller.

Producing the cell culture product in a cell culture composition may include contacting the cell culture composition with a water-immiscible solvent. In some instances, the cell culture product is a water-immiscible compound. The water-immiscible solvent may be added to the cell culture composition to a final concentration of water-immiscible solvent of from about 0.5% (v/v) to about 50% (v/v) e.g., about 0.5% (v/v) to about 10% (v/v), about 0.5% (v/v) to about 20% (v/v), about 0.5% (v/v) to about 30% (v/v), about 0.5% to about 40% (v/v), about 40% (v/v) to about 50% (v/v), about 30% (v/v) to about 50% (v/v), about 20% (v/v) to about 50% (v/v), about 10% (v/v) to about 50% (v/v), or about 1% (v/v) to about 5% (v/v)). In some embodiments, the water-immiscible solvent is added to the cell culture composition to a final concentration of water-immiscible solvent of from about 5% (v/v) to about 25% (v/v).

The water-immiscible may have a log(K_{d}) value of from about 1 to about 15, where K_{d} is the partition coefficient for the cell culture product between the water-immiscible solvent and the cell culture composition. For example, the log (K_{d}) may be about 1.5, about 1.6, about 1.7, about 1.8, about 1.9, about 2, about 2.1, about 2.2, about 2.3, about 2.4, about 2.5, about 2.6, about 2.7, about 2.8, about 2.9, or about 3. In some embodiments, the water-immiscible solvent may log(K_{d}) value of from about 2 to about 3. For example, the water-immiscible solvent has a log(K_{d}) value of about 2, about 2.1, about 2.2, about 2.3, about 2.4, about 2.5, about 2.6, about 2.7, about 2.8, about 2.9, or about 3.

The cell culture product may have a log(D) value of from about 1 to about 15 (e.g., about 1.1, 1.2, 1.3, 1.4, 1.5, 1.6, 1.7, 1.8, 1.9, 2, 2.1, 2.2, 2.3, 2.4, 2.5, 2.6, 2.7 2.8, 2.9, 3.1, 3.2, 3.3, 3.4, 3.5, 3.6, 3.7, 3.8, 3.9, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.22, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, or 15).

The water-immiscible solvent may be an alcohol. For example, the water-immiscible solvent may be a C₁₀-C₂₀ alcohol (e.g., C₁₀ alcohol, C₁₁ alcohol, C₁₂ alcohol, C₁₃ alcohol, C₁₄ alcohol, C₁₅ alcohol, C₁₆ alcohol, C₁₇ alcohol, C₁₈ alcohol, C₁₉ alcohol, or C₂₀ alcohol). In some embodiments, the water-immiscible solvent may be a C₁₂-C₁₈ alcohol (e.g., C₁₂ alcohol, C₁₃ alcohol, C₁₄ alcohol, C₁₅ alcohol, C₁₆ alcohol, C₁₇ alcohol, or C₁₈ alcohol). In some embodiments, the water-immiscible solvent is a vegetable oil. In some embodiments, the water-immiscible solvent is corn oil, sunflower oil, soybean oil, mineral oil, polyalphaolefin, dodecane, hexadecane, oleyl alcohol, butyl oleate, dibutyl phthalate, dodecanol, dioctyl phthalate, farnesene, or isopropyl myristate. In some embodiments, the water-immiscible solvent is a Drakeol™ fluid. In some embodiments, the water-immiscible solvent is a Jarcol™ fluid. In some embodiments, the water-immiscible solvent is a Durasyn™ fluid.

In some embodiments, cell culture product may be isolated from the cell culture composition using a filter, optionally wherein the filter is located in the vessel. In some embodiments, the filter separates the water-immiscible solvent from the cell culture composition in the vessel. In some embodiments, the filter is capable of separating at least a portion of the cell culture product from at least a portion of the host cells in the vessel. In some embodiments, the vessel is a fermentation vessel.

### Enzymes of Exemplary Biosynthetic Pathways

The host cells described herein may express one or more enzymes of a biosynthetic pathway capable of producing a cell culture product (e.g., fermentation product) of interest. In some embodiments, for example, host cells of the disclosure may naturally express some of the enzymes of the biosynthetic pathway for a given isoprenoid. Such host cells may be modified to express the remaining or heterologous enzymes of the biosynthetic pathway. In some embodiments, for instance, a host cell may naturally express many of the enzymes of the biosynthetic pathway of a desired isoprenoid (e.g., a terpene), and the host cells may be modified so as to express the remaining enzymes of the biosynthetic pathway for the desired isoprenoid by providing the cells with one or more heterologous nucleic acid molecules that, together, encode the remaining enzymes of the biosynthetic pathway. The one or more enzymes may be from the mevalonate-dependent (MEV) pathway or the 1-deoxy-D-xylulose 5-diphosphate (DXP) pathway. In some embodiments, the host cell is a yeast cell.

The host cells described herein may be modified to express one or more enzymes of the MEV biosynthetic pathway. Host cells which are modified with one or more enzymes of the MEV biosynthetic pathway may be capable of an increased production of one or more isoprenoid compounds as compared to host cell which is not modified with one or enzymes of the MEV biosynthetic pathway.

In some embodiments, the isoprenoid producing cell comprises a heterologous nucleotide sequence encoding an enzyme that can condense two molecules of acetyl-coenzyme A to form acetoacetyl-CoA, e.g., an acetyl-CoA thiolase. Illustrative examples of nucleotide sequences encoding such an enzyme include but are not limited to: (NC_000913 REGION: 2324131.2325315; Escherichia coli), (D49362; Paracoccus denitrifzcans), and (L20428; Saccharomyces cerevisiae).

In some embodiments, the host cell includes a heterologous nucleotide sequence encoding an enzyme that can condense acetoacetyl-CoA with another molecule of acetyl-CoA to form 3-hydroxy-3-methylglutaryl-CoA (HMG-CoA), e.g., a HMGCoA synthase. Illustrative examples of nucleotide sequences encoding such an enzyme include but are not limited to: (NC_001145. complement 19061.20536; Saccharomyces cerevisiae), (X96617; Saccharomyces cerevisiae), (X83882; Arabidopsis thaliana), (AB037907; Kitasatospora griseola), (BT007302; Homo sapiens), and (NC_002758, Locus tag SAV2546, GeneID 1122571; Staphylococcus aureus).

In some embodiments, the host cell includes a heterologous nucleotide sequence encoding an enzyme that can convert HMG-CoA into mevalonate, e.g., an HMG-CoA reductase. Illustrative examples of nucleotide sequences encoding such an enzyme include, but are not limited to: (NM_206548; Drosophila melanogaster), (NC_002758, Locus tag SAV2545, GeneID 1122570; Staphylococcus aureus), (NM_204485; Gallus gallus), (AB015627; Streptomyces sp. KO 3988), (AF542543; Nicotiana attenuata), (AB037907; Kitasatospora griseola), (AX128213, providing the sequence encoding a truncated HMGR; Saccharomyces cerevisiae), and (NC_001145: complement (115734.118898; Saccharomyces cerevisiae).

In some embodiments, the host cells include a heterologous nucleotide sequence encoding an enzyme that can convert mevalonate into mevalonate 5-phosphate, e.g., a mevalonate kinase. Illustrative examples of nucleotide sequences encoding such an enzyme include but are not limited to: (L77688; Arabidopsis thaliana), and (X55875; Saccharomyces cerevisiae).

In some embodiments, the host cells include a heterologous nucleotide sequence encoding an enzyme that can convert mevalonate 5-phosphate into mevalonate 5-pyrophosphate, e.g., a phosphomevalonate kinase. Illustrative examples of nucleotide sequences encoding such an enzyme include but are not limited to: (Af 429385; Hevea brasiliensis), (NM_006556; Homo sapiens), and (NC_001 145. Complement 712315.713670; Saccharomyces cerevisiae).

In some embodiments, the host cells include a heterologous nucleotide sequence encoding an enzyme that can convert mevalonate 5-pyrophosphate into isopentenyl diphosphate (IPP), e.g., a mevalonate pyrophosphate decarboxylase. Illustrative examples of nucleotide sequences encoding such an enzyme include but are not limited to: (X97557; Saccharomyces cerevisiae), (AF290095; Enterococcus faecium), and (U49260; Homo sapiens).

In some embodiments, the host cells include one or more heterologous nucleotide sequences encoding more than one enzyme of the MEV pathway. In some embodiments, the host cell includes one or more heterologous nucleotide sequences encoding two enzymes of the MEV pathway. In some embodiments, the host cell includes one or more heterologous nucleotide sequences encoding an enzyme that can convert HMG-CoA into mevalonate and an enzyme that can convert mevalonate into mevalonate 5-phosphate. In some embodiments, the host cell includes one or more heterologous nucleotide sequences encoding three enzymes of the MEV pathway. In some embodiments, the host cell includes one or more heterologous nucleotide sequences encoding four enzymes of the MEV pathway. In some embodiments, the host cell includes one or more heterologous nucleotide sequences encoding five enzymes of the MEV pathway. In some embodiments, the host cell includes one or more heterologous nucleotide sequences encoding six enzymes of the MEV pathway.

In some embodiments, the host cell further includes a heterologous nucleotide sequence encoding an enzyme that can convert IPP generated via the MEV pathway into its isomer, dimethylallyl pyrophosphate (DMAPP). DMAPP can be condensed and modified through the action of various additional enzymes to form simple and more complex isoprenoids.

The host cells described herein may be modified to express one or more enzymes of the DXP biosynthetic pathway. Host cells which are modified with one or more enzymes of the DXP biosynthetic pathway may be capable of an increased production of one or more isoprenoid compounds as compared to host cell which is not modified with one or enzymes of the DXP biosynthetic pathway.

In some embodiments, the host cells include a heterologous nucleotide sequence encoding an enzyme that can condense two molecules of acetyl-coenzyme A to form acetoacetyl-CoA, e.g., an acetyl-CoA thiolase. Illustrative examples of nucleotide sequences encoding such an enzyme include but are not limited to: (NC_000913 REGION: 2324131.2325315; Escherichia coli), (D49362; Paracoccus denitrifzcans), and (L20428; Saccharomyces cerevisiae).

In some embodiments, the host cell includes a heterologous nucleotide sequence encoding an enzyme, e.g., 1-deoxy-D-xylulose-5-phosphate synthase, which can condense pyruvate with D-glyceraldehyde 3-phosphate to make 1-deoxy-D-xylulose-5-phosphate. Illustrative examples of nucleotide sequences encoding such an enzyme include but are not limited to: (AF035440; Escherichia coli), (NC_002947, locus tag PP0527; Pseudomonas putida KT2440), (CP000026, locus tag SPA2301; Salmonella enterica Paratyphi, see ATCC 9150), (NC_007493, locus tag RSP_0254; Rhodobacter sphaeroides 2.4.1), (NC_005296, locus tag RP A0952; Rhodopseudomonas palustris CGA009), (NC_004556, locus tag PD1293; Xylellafastidiosa Temecula]), and (NC_003076, locus tag AT5GI 1380; Arabidopsis thaliana).

In some embodiments, the host cell includes a heterologous nucleotide sequence encoding an enzyme, e.g., 1-deoxy-D-xylulose-5-phosphate reductoisomerase, which can convert 1-deoxy-D-xylulose-5-phosphate to 2C-methyl-Derythritol-4-phosphate. Illustrative examples of nucleotide sequences include but are not limited to: (AB013300; Escherichia coli), (AF148852; Arabidopsis thaliana), (NC_002947, locus tag PP1597; Pseudomonas putida KT2440), (AL939124, locus tag SC05694; Streptomyces coelicolor A3(2)), (NC_007493, locus tag RSP 2709; Rhodobacter sphaeroides 2.4.1), and (NC_007492, locus tag Pfl_I 107; Pseudomonas jluorescens PfO-1).

In some embodiments, the host cell includes a heterologous nucleotide sequence encoding an enzyme, e.g., 4-diphosphocytidyl-2C-methyl-D-erythritol synthase, which can convert 2C-methyl-D-erythritol-4-phosphate to 4-diphosphocytidyl-2Cmethyl-D-erythritol. Illustrative examples of nucleotide sequences include but are not limited to: (AF230736; Escherichia coli), (NC_007493, locus tag RSP 2835; Rhodobacter sphaeroides 2.4.1), (NC_003071, locus tag AT2G02500; Arabidopsis thaliana), and (NC_002947, locus tag PP1614; Pseudomonas putida KT2440).

In some embodiments, the host cell includes a heterologous nucleotide sequence encoding an enzyme, e.g., 4-diphosphocytidyl-2C-methyl-D-erythritol kinase, which can convert 4-diphosphocytidyl-2C-methyl-D-erythritol to 4-diphosphocytidyl-2C-methyl-D-erythritol-2-phosphate. Illustrative examples of nucleotide sequences include but are not limited to: (AF216300; Escherichia coli) and (NC_007493, locus tag RSP 1779; Rhodobacter sphaeroides 2.4.1).

In some embodiments, the host cell includes a heterologous nucleotide sequence encoding an enzyme, 2C-methyl-D-erythritol 2,4-cyclodiphosphate synthase, which can convert 4-diphosphocytidyl-2C-methyl-D-erythritol-2-phosphate to 2Cmethyl-D-erythritol 2,4-cyclodiphosphate. Illustrative examples of nucleotide sequences include but are not limited to: (AF230738; Escherichia coli), (NC_007493, locus tag RSP_6071; Rhodobacter sphaeroides 2.4.1), and (NC_002947, locus tag PP1618; Pseudomonas putida KT2440).

In some embodiments, the host cell includes a heterologous nucleotide sequence encoding an enzyme, e.g., 1-hydroxy-2-methyl-2-(E)-butenyl-4-diphosphate synthase, which can convert 2C-methyl-D-erythritol 2,4-cyclodiphosphate to 1-hydroxy-2-methy 1-2-(E)-butenyl-4-di phosphate. Illustrative examples of nucleotide sequences include but are not limited to: (AY033515; Escherichia coli), (NC_002947, locus tag PP0853; Pseudomonas putida KT2440), and (NC_007493, locus tag RSP 2982; Rhodobacter sphaeroides 2.4.1).

In some embodiments, the host cell includes a heterologous nucleotide sequence encoding an enzyme, e.g., isopentyl/dimethylallyl diphosphate synthase, which can convert 1-hydroxy-2-methyl-2-(E)-butenyl-4-diphosphate into either IPP or its isomer, DMAPP. Illustrative examples of nucleotide sequences include but are not limited to: (AY062212; Escherichia coli) and (NC_002947, locus tag PP0606; Pseudomonas putida KT2440).

In some embodiments, the host cell includes one or more heterologous nucleotide sequences encoding more than one enzyme of the DXP pathway. In some embodiments, the host cell includes one or more heterologous nucleotide sequences encoding two enzymes of the DXP pathway. In some embodiments, the host cell includes one or more heterologous nucleotide sequences encoding three enzymes of the DXP pathway. In some embodiments, the host cell includes one or more heterologous nucleotide sequences encoding four enzymes of the DXP pathway. In some embodiments, the host cell includes one or more heterologous nucleotide sequences encoding five enzymes of the DXP pathway. In some embodiments, the host cell includes one or more heterologous nucleotide sequences encoding six enzymes of the DXP pathway. In some embodiments, the host cell includes one or more heterologous nucleotide sequences encoding five enzymes of the DXP pathway. In some embodiments, the host cell includes one or more heterologous nucleotide sequences encoding seven enzymes of the DXP pathway.

In some embodiments, “cross talk” (or interference) between the host cell's own metabolic processes and those processes involved with the production of IPP are minimized or eliminated entirely. For example, cross talk is minimized or eliminated entirely when the host cell relies exclusively on the DXP pathway for synthesizing IPP, and a MEV pathway is introduced to provide additional IPP. Such a host cell would not be equipped to alter the expression of the MEV pathway enzymes or process the intermediates associated with the MEV pathway. Organisms that rely exclusively or predominately on the DXP pathway include, for example, Escherichia coli.

In some embodiments, the host cell produces IPP via the MEV pathway, either exclusively or in combination with the DXP pathway. In other embodiments, a host cell's DXP pathway is functionally disabled so that the host cell produces IPP exclusively through a heterologously introduced MEV pathway. The DXP pathway can be functionally disabled by disabling gene expression or inactivating the function of one or more of the DXP pathway enzymes.

In some embodiments, the host cell further includes a heterologous nucleotide sequence encoding a polyprenyl synthase that can condense IPP and/or DMAPP molecules to form polyprenyl compounds containing more than five carbons.

In some embodiments, the isoprenoid producing cell further comprises a heterologous nucleotide sequence encoding an enzyme that can convert IPP generated via the MEV pathway into DMAPP, e.g., an IPP isomerase. Illustrative examples of nucleotide sequences encoding such an enzyme include but are not limited to: (NC_000913, 3031087.3031635; Escherichia coli), and (AF082326; Haematococcus pluvialis).

In some embodiments, the host cell includes a heterologous nucleotide sequence encoding an enzyme that can condense one molecule of IPP with one molecule of DMAPP to form one molecule of geranyl pyrophosphate (GPP), e.g., a GPP synthase. Illustrative examples of nucleotide sequences encoding such an enzyme include, but are not limited to: (AF513III; Abies grandis), (AF513112; Abies grandis), (AF513113; Abies grandis), (AY534686; Antirrhinum majus), (AY534687; Antirrhinum majus), (YI 7376; Arabidopsis thaliana), (AE016877, Locus API 1092; Bacillus cereus; ATCC 14579), (AJ243739; Citrus sinensis), (AY534745; Clarkia breweri), (AY953508; fps pini), (DQ286930; Lycopersicon esculentum), (AF182828; Mentha x piperita), (AF182827; Mentha x piperita), (MP1249453; Mentha x piperita), (PZE431697, Locus CAD24425; Paracoccus zeaxanthinifaciens), (A Y866498; Picrorhiza kurrooa), (A Y35 I 862; Vitis vinifera), and (AF203881, Locus AAF12843; Zymomonas mobilis).

In some embodiments, the host cell includes a heterologous nucleotide sequence encoding an enzyme that can condense two molecules of IPP with one molecule of DMAPP, or add a molecule of IPP to a molecule of GPP, to form a molecule of farnesyl pyrophosphate (FPP), e.g., a FPP synthase. Illustrative examples of nucleotide sequences that encode such an enzyme include, but are not limited to: (ATU80605; Arabidopsis thaliana), (ATHFPS2R; Arabidopsis thaliana), (AAU36376; Artemisia annua), (AF461050; Bos taurus), (D00694; Escherichia coli K-12), (AE009951, Locus AAL95523; Fusobacterium nucleatum subsp. nucleatum ATCC 25586), (GFFPPSGEN; Gibberella Jujikuroi), (CP000009, Locus AAW60034; Gluconobacter oxydans 621H), (AF019892; Helianthus annuus), (HUMP APS; Homo sapiens), (KLPFPSQCR; Kluyveromyces lactis), (LAU15777; Lupinus albus), (LAU20771; Lupinus albus), (AF309508; Mus musculus), (NCFPPSGEN; Neurospora crassa), (PAFPSI; Parthenium argentatum), (PAFPS2; Parthenium argentatum), (RA TF APS; Rattus norvegicus), (YSCFPP; Saccharomyces cerevisiae), (D89104; SchizoSaccharomyces pombe), (CP000003, Locus AAT87386; Streptococcus pyogenes), (CP0000I 7, Locus AAZ51849; Streptococcus pyogenes), (NC_008022, Locus YP 598856; Streptococcus pyogenes MGAS 10270), (NC_008023, Locus YP 600845; Streptococcus pyogenes MGAS2096), (NC_008024, Locus YP 602832; Streptococcus pyogenes MGAS10750), (MZEFPS; Zea mays), (AE000657, Locus AAC06913; Aquifex aeolicus VF5), (NM_202836; Arabidopsis thaliana), (D84432, Locus BAA12575; Bacillus subtilis), (U12678, Locus AAC28894; Bradyrhizobium japonicum USDA 110), (BACFDPS; Geobacillus stearothermophilus), (NC_002940, Locus NP 873754; Haemophilus ducreyi 35000HP), (L42023, Locus AAC23087; Haemophilus injluenzae Rd KW20), (J05262; Homo sapiens), (YP 395294; Lactobacillus sakei subsp. sakei 23K), (NC_005823, Locus YP 000273; Leptospira interrogans serovar Copenhageni str. Fiocruz LI-130), (AB003187; Micrococcus luteus), (NC_002946, Locus YP_208768; Neisseria gonorrhoeae FA 1090), (U00090, Locus AAB91752; Rhizobium sp. NGR234), (J05091; Saccharomyces cerevisiae), (CP000031, Locus AAV93568; Silicibacter pomeroyi DSS-3), (AE008481, Locus AAK99890; Streptococcus pneumoniae R6), and (NC_004556, Locus NP 779706; Xylella fastidiosa Temecula1).

In some embodiments, the host cell includes a heterologous nucleotide sequence encoding an enzyme that can combine IPP and DMAPP or IPP and FPP to form geranylgeranyl pyrophosphate (GGPP). Illustrative examples of nucleotide sequences that encode such an enzyme include, but are not limited to: (ATHGERPYRS; Arabidopsis thaliana), (BT005328; Arabidopsis thaliana), (NM_I 19845; Arabidopsis thaliana), (NZ_AAJM01000380, Locus ZP 00743052; Bacillus thuringiensis serovar israelensis, ATCC 35646 sql563), (CRGGPPS; Catharanthus roseus), (NZ_AABF02000074, Locus ZP 00144509; Fusobacterium nucleatum subsp. vincentii, ATCC 49256), (GFGGPPSGN; Gibberella fujikuroi), (AY371321; Ginkgo biloba), (AB055496; Hevea brasiliensis), (AB0I 7971; Homo sapiens), (MC1276129; Mucor circinelloides f. lusitanicus), (AB016044; Mus musculus), (AABX01000298, Locus NCU01427; Neurospora crassa), (NCU20940; Neurospora crassa), (NZ_AAKL01000008, Locus ZP 00943566; Ralstonia solanacearum UW551), (ABI 18238; Rattus norvegicus), (SCU31632; Saccharomyces cerevisiae), (AB016095; Synechococcus elongates), (SAGGPS; Sinapis alba), (SSOGDS; Sulfolobus acidocaldarius), (NC_007759, Locus YP 461832; Syntrophus aciditrophicus SB), (NC_006840, Locus YP 204095; Vibrio jischeri ESI 14), (NM_112315; Arabidopsis thaliana), (ERWCR TE; Pantoea agglomerans), (D90087, Locus BAA14124; Pantoea ananatis), (X52291, Locus CAA36538; Rhodobacter capsulatus), (AF195122, Locus AAF24294; Rhodobacter sphaeroides), and (NC_004350, Locus NP 721015; Streptococcus mutans UA159).

In some embodiments, the host cell further includes a heterologous nucleotide sequence encoding an enzyme that can modify a polyprenyl to form a hemiterpene, a monoterpene, a sesquiterpene, a diterpene, a triterpene, a tetraterpene, a polyterpene, a steroid compound, a carotenoid, or a modified isoprenoid compound.

In some embodiments, the heterologous nucleotide encodes a carene synthase. Illustrative examples of suitable nucleotide sequences include, but are not limited to: (AF461460, REGION 43.1926; Picea abies) and (AF527416, REGION: 78.1871; Salvia stenophylla).

In some embodiments, the heterologous nucleotide encodes a geraniol synthase. Illustrative examples of suitable nucleotide sequences include, but are not limited to: (Af 457070; Cinnamomum tenuipilum), (A Y362553; Ocimum basilicum), (DQ234300; Perilla frutescens strain 1864), (DQ234299; Perilla citriodora strain 1861), (DQ234298; Perilla citriodora strain 4935), and (DQ088667; Perilla citriodora).

In some embodiments, the heterologous nucleotide encodes a linalool synthase. Illustrative examples of a suitable nucleotide sequence include, but are not limited to: (AF497485; Arabidopsis thaliana), (AC002294, Locus AAB71482; Arabidopsis thaliana), (AY059757; Arabidopsis thaliana), (NM_104793; Arabidopsis thaliana), (AF154124; Artemisia annua), (AF067603; Clarkia breweri), (AF067602; Clarkia concinna), (AF067601; Clarkia breweri), (U58314; Clarkia breweri), (AY840091; Lycopersicon esculentum), (DQ263741; Lavandula angustifolia), (AY083653; Mentha citrate), (AY693647; Ocimum basilicum), (XM_463918; Oryza sativa), (AP004078, Locus BAD07605; Oryza sativa), (XM_463918, Locus XP_463918; Oryza sativa), (AY917193; Perilla citriodora), (AF271259; Perilla frutescens), (AY473623; Picea abies), (DQ195274; Picea sitchensis), and (AF444798; Perilla frutescens var. crispa cultivar No. 79).

In some embodiments, the heterologous nucleotide encodes a limonene synthase. Illustrative examples of suitable nucleotide sequences include but are not limited to: (+)limonene synthases (AF514287, REGION: 47.1867; Citrus limon) and (AY055214, REGION: 48.1889; Agastache rugosa) and (−)-limonene synthases (DQ195275, REGION: 1.1905; Picea sitchensis), (AF006193, REGION: 73.1986; Abies grandis), and (MHC4SLSP, REGION: 29.1828; Mentha spicata).

In some embodiments, the heterologous nucleotide encodes a myrcene synthase. Illustrative examples of suitable nucleotide sequences include, but are not limited to: (U87908; Abies grandis), (A YI 95609; Antirrhinum majus), (A YI 95608; Antirrhinum majus), (NM_127982; Arabidopsis thaliana TPSIO), (NM_113485; Arabidopsis thaliana ATTPS-CIN), (NM_113483; Arabidopsis thaliana ATTPS-CIN), (AF271259; Perilla frutescens), (AY473626; Picea abies), (AF369919; Picea abies), and (AJ304839; Quercus ilex).

In some embodiments, the heterologous nucleotide encodes an ocimene synthase. Illustrative examples of suitable nucleotide sequences include, but are not limited to: (AYI 95607; Antirrhinum majus), (A YI 95609; Antirrhinum majus), (A YI 95608; Antirrhinum majus), (AK221024; Arabidopsis thaliana), (NM_113485; Arabidopsis thaliana ATTPS-CIN), (NM_113483; Arabidopsis thaliana ATTPS-CIN), (NM_II 7775; Arabidopsis thaliana ATTPS03), (NM_001036574; Arabidopsis thaliana ATTPS03), (NM_127982; Arabidopsis thaliana TIPS 10), (AB 110642; Citrus unshiu CitMTSL4), and (AY575970; Lotus corniculatus var. Japonicus).

In some embodiments, the heterologous nucleotide encodes an α-pinene synthase. Illustrative examples of suitable nucleotide sequences include but are not limited to: (+) apinene synthase (AF543530, REGION: 1.1887; Pinus taeda), (−)α-pinene synthase (AF543527, REGION: 32.1921; Pinus taeda), and (+)/(−)α-pinene synthase (AGU87909, REGION: 6111892; Abies grandis).

In some embodiments, the heterologous nucleotide encodes a P-pinene synthase. Illustrative examples of suitable nucleotide sequences include but are not limited to: (−) Ppinene synthases (AF276072, REGION: 1.1749; Artemisia annua) and (AF514288, REGION: 26.1834; Citrus limon).

In some embodiments, the heterologous nucleotide encodes a sabinene synthase. An illustrative example of a suitable nucleotide sequence includes but is not limited to AF05| 901, REGION: 26.1798 from Salvia ofjicinalis.

In some embodiments, the heterologous nucleotide encodes a y-terpinene synthase. Illustrative examples of suitable nucleotide sequences include, but are not limited to: (AF514286, REGION: 30.1832 from Citrus limon) and (ABI 10640, REGION 1.1803 from Citrus unshiu).

In some embodiments, the heterologous nucleotide encodes a terpinolene synthase. Illustrative examples of a suitable nucleotide sequence include but are not limited to: (AY693650 from Ocimum basilicum) and (AY906866, REGION: 10.1887 from Pseudotsuga menziesii).

In some embodiments, the heterologous nucleotide encodes an amorphadiene synthase. An illustrative example of a suitable nucleotide sequence is SEQ ID NO. 37 of U.S. Patent Publication No. 2004/0005678.

In some embodiments, the heterologous nucleotide encodes an a-farnesene synthase. Illustrative examples of suitable nucleotide sequences include but are not limited to DQ309034 from Pyrus communis cultivar d'Anjou (pear; gene name AFSI) and AY182241 from Malus domestica (apple; gene AFSI). Pechouus et al., Planta 219(1):84-94 (2004).

In some embodiments, the heterologous nucleotide encodes a P-farnesene synthase. Illustrative examples of suitable nucleotide sequences include but are not limited to GenBank accession number AF024615 from Mentha x piperita (peppermint; gene Tspal 1), and A Y835398 from Artemisia annua. Picaud et al., Phytochemistry 66(9): 961-967 (2005).

In some embodiments, the heterologous nucleotide encodes a farnesol synthase. Illustrative examples of suitable nucleotide sequences include but are not limited to GenBank accession number AF529266 from Zea mays and YDR481C from Saccharomyces cerevisiae (gene Pho8). Song, L., Applied Biochemistry and Biotechnology 128: 149-15 8 (2006).

In some embodiments, the heterologous nucleotide encodes a nerolidol synthase. An illustrative example of a suitable nucleotide sequence includes but is not limited to AF529266 from Zea mays (maize; gene tpsl).

In some embodiments, the heterologous nucleotide encodes a patchoulol synthase. Illustrative examples of suitable nucleotide sequences include but are not limited to AY508730 REGION: 1.1659 from Pogostemon cablin.

In some embodiments, the heterologous nucleotide encodes a nootkatone synthase. Illustrative examples of a suitable nucleotide sequence include but are not limited to AF441124 REGION: 1.1647 from Citrus sinensis and AY917195 REGION: 1.1653 from Perilla frutescens.

In some embodiments, the heterologous nucleotide encodes an abietadiene synthase.

### Illustrative Examples of Suitable Nucleotide Sequences

In some embodiments, one or more heterologous nucleic acids encoding one or more enzymes are integrated into the genome of the host cell. In some embodiments, one or more heterologous nucleic acids encoding one or more enzymes are present within one or more plasmids.

In some embodiments, for example, host cells of the disclosure (e.g., yeast cells) may naturally express some of the enzymes of the biosynthetic pathway for a given cannabinoid. Such host cells may be modified to express the remaining or heterologous enzymes of the biosynthetic pathway. In some embodiments, for instance, a host cell (e.g., a yeast cell) may naturally express many of the enzymes of the biosynthetic pathway of a desired cannabinoid, and the host cells may be modified so as to express the remaining enzymes of the biosynthetic pathway for the desired cannabinoid by providing the cells with one or more heterologous nucleic acid molecules that, together, encode the remaining enzymes of the biosynthetic pathway.

In some embodiments, the host cell includes a heterologous genetic pathway that produces a cannabinoid or a precursor of a cannabinoid. The cannabinoid biosynthetic pathway may begin with hexanoic acid as the substrate for an acyl activating enzyme (AAE) to produce hexanoyl-CoA, which is used as the substrate of a tetraketide synthase to produce tetraketide-CoA, which is used by an olivetolic acid cyclase (OAC) to produce olivetolic acid, which is then used to produce a cannabigerolic acid by a geranyl pyrophosphate (GPP) synthase and a cannabigerolic acid synthase (CBGaS). In some embodiments, the cannabinoid precursor that is produced is a substrate in the cannabinoid pathway (e.g., hexanoate or olivetolic acid). In some embodiments, the precursor is a substrate for an AAE, a TKS, an OAC, a CBGaS, or a GPP synthase. In some embodiments, the precursor, substrate, or intermediate in the cannabinoid pathway is hexanoate, olivetol, or olivetolic acid. In some embodiments, the precursor is hexanoate. In some embodiments, the host cell does not contain the precursor, substrate or intermediate in an amount sufficient to produce the cannabinoid or a precursor of the cannabinoid. In some embodiments, the heterologous genetic pathway encodes at least one enzyme selected from the group consisting of an AAE, a TKS, an OAC, a CBGaS, or a GPP synthase. In some embodiments, the genetically modified host cell includes an AAE, TKS, OAC, CBGaS, and a GPP synthase. The cannabinoid pathway is described in Keasling et al. (WO 2018/200888).

### Culture and Fermentation Methods

Materials and methods for the maintenance and growth of microbial cultures are well known to those skilled in the art of microbiology or fermentation science (see, for example, Bailey et al., Biochemical Engineering Fundamentals, second edition, McGraw Hill, New York, 1986). Consideration must be given to appropriate culture medium, pH, temperature, and requirements for aerobic, microaerobic, or anaerobic conditions, depending on the specific requirements of the host cell, the fermentation, and the process.

The methods of producing a cell culture product (e.g., cell culture product), such as an isoprenoid or terpene, provided herein may be performed in a suitable culture medium in a suitable container, including but not limited to a cell culture plate, a flask, or a vessel, such as but not limited to a fermentation vessel. Further, the methods can be performed at any scale of fermentation known in the art to support industrial production of microbial products. Any suitable vessel may be used including a stirred tank fermentation vessel, an airlift fermentation vessel, a bubble fermentation vessel, or any combination thereof. In particular embodiments utilizing Saccharomyces cerevisiae as the host cell, strains can be grown in a fermentation vessel as described in detail by Kosaric, et al, in Ullmann's Encyclopedia of Industrial Chemistry, Sixth Edition, Volume 12, pages 398-473, Wiley-VCH Verlag GmbH & Co. KDaA, Weinheim, Germany.

In some embodiments, the culture medium is any culture medium in which a genetically modified microorganism capable of producing a heterologous product can subsist, i.e., maintain growth and viability. In some embodiments, the culture medium is an aqueous medium comprising assimilable carbon, nitrogen, and phosphate sources. Such a medium can also include appropriate salts, minerals, metals, and other nutrients. In some embodiments, the carbon source and each of the essential cell nutrients are added incrementally or continuously to the culture medium, and each required nutrient is maintained at essentially the minimum level needed for efficient assimilation by growing cells, for example, in accordance with a predetermined cell growth curve based on the metabolic or respiratory function of the cells which convert the carbon source to a biomass.

Suitable conditions and suitable medium for culturing microorganisms are well known in the art. In some embodiments, the suitable medium is supplemented with one or more additional agents, such as, for example, an overlay or other water-immiscible solvent. In other embodiments, the suitable medium is supplemented with antifoam. In some embodiments, the suitable medium is supplemented with an inducer (e.g., when one or more nucleotide sequences encoding a gene product are under the control of an inducible promoter), a repressor (e.g., when one or more nucleotide sequences encoding a gene product are under the control of a repressible promoter), or a selection agent (e.g., an antibiotic to select for microorganisms comprising the genetic modifications).

In some embodiments, the carbon source is a monosaccharide (simple sugar), a disaccharide, a polysaccharide, a non-fermentable carbon source, or one or more combinations thereof. Non-limiting examples of suitable monosaccharides include glucose, galactose, mannose, fructose, ribose, and combinations thereof. Non-limiting examples of suitable disaccharides include sucrose, lactose, maltose, trehalose, cellobiose, and combinations thereof. Non-limiting examples of suitable polysaccharides include starch, glycogen, cellulose, chitin, and combinations thereof. Non-limiting examples of suitable non-fermentable carbon sources include acetate, ethanol, and glycerol.

The concentration of a carbon source, such as glucose or sucrose, in the culture medium should promote cell growth, but not be so high as to repress growth of the microorganism used. Typically, cultures are run with a carbon source, such as glucose or sucrose, being added at levels to achieve the desired level of growth and biomass. Production of cell culture products, such as isoprenoids, may also occur in these culture conditions, but at undetectable levels (with detection limits being about <0.1 g/l). In other embodiments, the concentration of a carbon source, such as glucose or sucrose, in the culture medium is greater than about 1 g/L, preferably greater than about 2 g/L, and more preferably greater than about 5 g/L. In addition, the concentration of a carbon source, such as glucose or sucrose, in the culture medium is typically less than about 100 g/L, preferably less than about 50 g/L, and more preferably less than about 20 g/L. It should be noted that references to culture component concentrations can refer to both initial and/or ongoing component concentrations. In some cases, it may be desirable to allow the culture medium to become depleted of a carbon source during culture.

Sources of assimilable nitrogen that can be used in a suitable culture medium include, but are not limited to, simple nitrogen sources, organic nitrogen sources and complex nitrogen sources. Such nitrogen sources include anhydrous ammonia, ammonium salts and substances of animal, vegetable and/or microbial origin. Suitable nitrogen sources include, but are not limited to, protein hydrolysates, microbial biomass hydrolysates, peptone, yeast extract, ammonium sulfate, urea, and amino acids. Typically, the concentration of the nitrogen sources, in the culture medium is greater than about 0.1 g/L, preferably greater than about 0.25 g/L, and more preferably greater than about 1.0 g/L. Beyond certain concentrations, however, the addition of a nitrogen source to the culture medium is not advantageous for the growth of the microorganisms. As a result, the concentration of the nitrogen sources, in the culture medium is less than about 20 g/L, preferably less than about 10 g/L and more preferably less than about 5 g/L. Further, in some instances it may be desirable to allow the culture medium to become depleted of the nitrogen sources during culture.

The effective culture medium can contain other compounds such as inorganic salts, vitamins, trace metals, or growth promoters. Such other compounds can also be present in carbon, nitrogen, or mineral sources in the effective medium or can be added specifically to the medium.

The culture medium can also contain a suitable phosphate source. Such phosphate sources include both inorganic and organic phosphate sources. Preferred phosphate sources include, but are not limited to, phosphate salts such as mono or dibasic sodium and potassium phosphates, ammonium phosphate, and mixtures thereof. Typically, the concentration of phosphate in the culture medium is greater than about 1.0 g/L, preferably greater than about 2.0 g/L, and more preferably greater than about 5.0 g/L. Beyond certain concentrations, however, the addition of phosphate to the culture medium is not advantageous for the growth of the microorganisms. Accordingly, the concentration of phosphate in the culture medium is typically less than about 20 g/L, preferably less than about 15 g/L, and more preferably less than about 10 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, preferably greater than about 1.0 g/L, and more preferably greater than about 2.0 g/L. Beyond certain concentrations, however, the addition of magnesium to the culture medium is not advantageous for the growth of the microorganisms. Accordingly, the concentration of magnesium in the culture medium is typically less than about 10 g/L, preferably less than about 5 g/L, and more preferably less than about 3 g/L. Further, in some instances, it may be desirable to allow the culture medium to become depleted of a magnesium source during culture.

In some embodiments, the culture medium can also include a biologically acceptable chelating agent, such as the dihydrate of trisodium citrate. In such instances, the concentration of a chelating agent in the culture medium is greater than about 0.2 g/L, preferably greater than about 0.5 g/L, and more preferably greater than about 1 g/L. Beyond certain concentrations, however, the addition of a chelating agent to the culture medium is not advantageous for the growth of the microorganisms. Accordingly, the concentration of a chelating agent in the culture medium is typically less than about 10 g/L, preferably less than about 5 g/L, and more preferably less than about 2 g/L.

The culture medium can also initially include a biologically acceptable acid or base to maintain the desired pH of the culture medium. Biologically acceptable acids include, but are not limited to, hydrochloric acid, sulfuric acid, nitric acid, phosphoric acid, and mixtures thereof. Biologically acceptable bases include, but are not limited to, ammonium hydroxide, sodium hydroxide, potassium hydroxide, and mixtures thereof. In some embodiments, the base used is ammonium hydroxide.

The culture medium can also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, preferably within the range of from about 20 mg/L to about 1000 mg/L, and more preferably in the range of from about 50 mg/L to about 500 mg/L.

The culture medium can also include sodium chloride. Typically, the concentration of sodium chloride in the culture medium is within the range of from about 0.1 g/L to about 5 g/L, preferably within the range of from about 1 g/L to about 4 g/L, and more preferably in the range of from about 2 g/L to about 4 g/L.

In some embodiments, the culture medium can also include trace metals. Such trace metals can be added to the culture medium as a stock solution that, for convenience, can be prepared separately from the rest of the culture medium. Typically, the amount of such a trace metals solution added to the culture medium is greater than about 1 mL/L, preferably greater than about 5 mL/L, and more preferably greater than about 10 mL/L. Beyond certain concentrations, however, the addition of trace metals to the culture medium is not advantageous for the growth of the microorganisms. Accordingly, the amount of such a trace metals solution added to the culture medium is typically less than about 100 mL/L, preferably less than about 50 mL/L, and more preferably less than about 30 mL/L. It should be noted that, in addition to adding trace metals in a stock solution, the individual components can be added separately, each within ranges corresponding independently to the amounts of the components dictated by the above ranges of the trace metals solution.

The culture medium can include other vitamins, such as pantothenate, biotin, calcium pantothenate, inositol, para-aminobenzoic acid, nicotinic acid, pyridoxine-HCl, and thiamine-HCl. Such vitamins can be added to the culture medium as a stock solution that, for convenience, can be prepared separately from the rest of the culture medium. Beyond certain concentrations, however, the addition of vitamins to the culture medium is not advantageous for the growth of the microorganisms.

The culture medium may include trace metals, such as iron, copper, cobalt, zinc, selenium, chromium, iodine, and molybdenum. Such trace metals can be added to the culture medium as a stock solution that, for convenience, can be prepared separately from the rest of the culture medium.

In some embodiments, the culture medium may include an antifoam or a surfactant. In some embodiments, the surfactant may be an anionic surfactant; for example, the surfactant may be alkyl-naphthalene sulfonate, alkyl benzene sulfonate, or the like. In some embodiments, the surfactant is a nonionic surfactant. Suitable surfactants include biocompatible nonionic surfactants such as Bri (e.g., polyoxyethylene (4) lauryl ether, also known as Brij-30; polyoxyethylene (2) oleyl ether; polyoxyethylene (2) stearyl ether; etc.); micelles; and the like. In some embodiments, the surfactant is a secondary ether polyol. In some embodiments, the surfactant is TERGITOL L-62 (Dow Chemical Company). In some embodiments, the surfactant is a Jarcol™ alcohol. In some embodiments, the surfactant is TERGAZYME (Alconox), which may be used in an amount of between 0% (w/v) and about 1% (w/v).

The cell culture methods described herein can be performed in conventional culture modes, which include, but are not limited to, batch, fed-batch, cell recycle, continuous and semi-continuous. In some embodiments, the cell culture is carried out in fed-batch mode. In such a case, some of the components of the medium are depleted during culture, including pantothenate during the production stage of the fermentation. In some embodiments, the culture may be supplemented with relatively high concentrations of such components at the outset, for example, of the production stage, so that growth and/or production is supported for a period of time before additions are required. The preferred ranges of these components are maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those in the art, the rate of consumption of nutrient increases during culture as the cell density of the medium increases. Moreover, to avoid introduction of foreign microorganisms into the culture medium, addition is performed using aseptic addition methods, as are known in the art. In addition, anti-foaming agent may be added during the culture.

The temperature of the culture medium can be any temperature suitable for growth of the genetically modified cells and/or production of compounds of interest. For example, prior to inoculation of the culture medium with an inoculum, the culture medium can be brought to and maintained at a temperature in the range of from about 20° C. to about 45° C., preferably to a temperature in the range of from about 25° C. to about 40° C. and more preferably in the range of from about 28° C. to about 32° C. In some embodiments, the culture medium can be brought to and maintain at a temperature in the range of from about 15° C. to about 50° C., preferably to a temperature in the range of from about 20° C. to about 45° C., and more preferably in the range of from about 30° C. to about 40° C.; for example, the temperature may be brought to and maintained at about 37° C.

The pH of the culture medium can be controlled by the addition of acid or base to the culture medium. In such cases when ammonia is used to control pH, it also conveniently serves as a nitrogen source in the culture medium. Preferably, the pH is maintained from about 3.0 to about 8.0, more preferably from about 3.5 to about 7.0, and most preferably from about 4.0 to about 6.5. In some embodiments, the pH is maintained from about 3.0 to about 9.0, more preferably from about 5 to about 8.5, and most preferably from about 6.0 to about 8.0.

In some embodiments, the carbon source concentration, such as the glucose concentration, of the culture medium is monitored during culture. Glucose or sucrose concentration of the culture medium can be monitored using known techniques, such as, for example, use of the glucose oxidase enzyme test or high-pressure liquid chromatography, which can be used to monitor glucose concentration in the supernatant, e.g., a cell-free component of the culture medium. As stated previously, the carbon source concentration should be kept below the level at which cell growth inhibition occurs. Although such concentration may vary from organism to organism, for glucose as a carbon source, cell growth inhibition occurs at glucose concentrations greater than at about 60 g/L and can be determined readily by trial. Accordingly, when glucose is used as a carbon source the glucose is preferably fed to the vessel and maintained below detection limits. Alternatively, the glucose concentration in the culture medium is maintained in the range of from about 1 g/L to about 100 g/L, more preferably in the range of from about 2 g/L to about 50 g/L, and yet more preferably in the range of from about 5 g/L to about 20 g/L. Although the carbon source concentration can be maintained within desired levels by addition of, for example, a substantially pure glucose solution, it is acceptable, and may be preferred, to maintain the carbon source concentration of the culture medium by addition of aliquots of the original culture medium. The use of aliquots of the original culture medium may be desirable because the concentrations of other nutrients in the medium (e.g., the nitrogen and phosphate sources) can be maintained simultaneously. Likewise, the trace metals concentrations can be maintained in the culture medium by addition of aliquots of the trace metals solution.

### Host Cell Strains

Any suitable host cell may be used in the practice of the present invention. Illustrative examples of suitable host cells include any archae, prokaryotic, or eukaryotic cell. Examples of an archae cell include, but are not limited to those belonging to the genera: Aeropyrum, Archaeglobus, Halobacterium, Methanococcus, Methanobacterium, Pyrococcus, Sulfolobus, and Thermoplasma. Illustrative examples of archae strains include but are not limited to: Aeropyrum pernix, Archaeoglobus fulgidus, Methanococcus jannaschii, Methanobacterium thermoautotrophicum, Pyrococcus abyssi, Pyrococcus horikoshii, Thermoplasma acidophilum, Thermoplasma volcanium.

Examples of a prokaryotic cell include, but are not limited to those belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Strepromyces, Synnecoccus, and Zymomonas. Illustrative examples of prokaryotic bacterial strains include but are not limited to: Bacillus subtilis, Bacillus amyloliquefacines, Brevibacterium ammoniagenes, Brevibacterium immariophilim, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, Staphylococcus aureus, and the like.

In general, if a bacterial host cel is used, a non-pathogenic strain is preferred. Illustrative examples of non-pathogenic strains include but are not limited to: Bacillus subtilis, Escherichia coli, Lactobacillus acidophilus, Lactobacillus helveticus, Pseudomonas aeruginosa, Pseudomonas mevalonii, Pseudomonas pudita, Rhodobacter sphaeroides, Rodobacter capsulatus, Rhodospirillum rubrum, and the like.

Examples of eukaryotic cells include but are not limited to fungal cells. Examples of fungal cell include, but are not limited to those belonging to the genera: Aspergillus, Candida, Chrysosporium, Cryptococcus, Fusarium, Kluyveromyces, Neotyphodium, Neurospora, Penicillium, Pichia, Saccharomyces, Trichoderma and Xanthophyllomyces (formerly Phaffia).

Illustrative examples of eukaryotic strains include but are not limited to: Aspergillus nidulans, Aspergillus niger, Aspergillus oryzae, Candida albicans, Chrysosporium lucknowense, Fusarium graminearum, Fusarium venenatum, Kluyveromyces lactis, Neurospora crassa, Pichia angusta, Pichia finlandica, Pichia kodamae, Pichia membranaefaciens, Pichia methanolica, Pichia opuntiae, Pichia pastoris, Pichia pijperi, Pichia quercuum, Pichia salictaria, Pichia thermotolerans, Pichia trehalophila, Pichia stipitis, Streptomyces ambofaciens, Streptomyces aureofaciens, Streptomyces aureus, Saccaromyces bayanus, Saccaromyces boulardi, Saccharomyces cerevisiae, Streptomyces fungicidicus, Streptomyces griseochromogenes, Streptomyces griseus, Streptomyces lividans, Streptomyces olivogriseus, Streptomyces rameus, Streptomyces tanashiensis, Streptomyces vinaceus, Trichoderma reesei and Xanthophyllomyces dendrorhous (formerly Phaffia rhodozyma).

In some embodiments of the present disclosure, the host cell is a yeast cell. Yeast cells useful in conjunction with the compositions and methods described herein include yeast that have been deposited with microorganism depositories (e.g. IFO, ATCC, etc.), such as those that belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, chizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In some embodiments, the strain is Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorphs (now known as Pichia angusta). In some embodiments, the host microbe is a strain of the genus Candida, such as Candida lipolytica, Candida guilliermondii, Candida krusei, Candida pseudotropicalis, or Candida utilis.

In a particular embodiment, the strain is Saccharomyces cerevisiae. In some embodiments, the host is a strain of Saccharomyces cerevisiae selected from the group consisting of Baker's yeast, CEN.PK, CEN.PK2, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, and AL-1. In some embodiments, the strain of Saccharomyces cerevisiae is CEN.PK. In some embodiments, the yeast strain used is Y21900. In some embodiments, the yeast strain used is Y23508.

In some embodiments, the strain is a microbe that is suitable for industrial fermentation. In particular embodiments, the microbe is conditioned to subsist under high solvent concentration, high temperature, expanded substrate utilization, nutrient limitation, osmotic stress due to sugar and salts, acidity, sulfite and bacterial contamination, or combinations thereof, which are recognized stress conditions of the industrial fermentation environment.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Continuous Separation of Farnesene Product from Fermentation Using Gravity Settling

In this Example, an inclined gravity settler was used to separate yeast cells in a fermentation composition from a water-immiscible terpene product, which in this instance was farnesene. It was important to remove the farnesene from the fermentation composition due to the fact that as the concentration of farnesene in the fermentation composition increases it becomes inhibitory. By separating the farnesene product from the yeast cells, the yeast cells may be returned to the vessel and farnesene may be removed.

There were several important factors to consider in order to achieve good separation of the fermentation composition including: (1) the yeast cells used, (2) the oil used, which includes the farnesene extracted from the aqueous-phase culture media, (3) the sedimentation rate of cells, and (4) the rising velocity of the farnesene/emulsion droplets. The sedimentation rate of a sphere is described by Stoke's Law when also considering a dense broth oriented at a fixed angle.

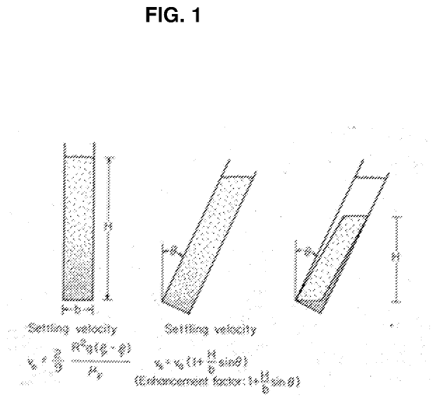

The general setup of a gravity settler that was used is described in FIG. 1. Aerated whole cell broth from a vessel (e.g., a fermentation vessel) was fed to a bubble trap where bubbles in the broth coalesced and burst. This bubble coalescence was important to the overall yield, as bubbles can disrupt the flow in the settling zone and reduce the efficicency of the separation. Bubble-free whole cell broth was then fed near the bottom of the gravity settler where the separation began. Most of the broth immediately returned to the vessel, but a small portion flowed toward the top of the device due to suction from the effluent pump. As broth flowed up the tube, the cells settled and were returned to the vessel while a majority of the farnesene oil and emulsion droplets rose to the top and out in the effluent. The effluent flow-rate was roughly the same as the syrup feed-rate in order to balance the weight of the vessel.

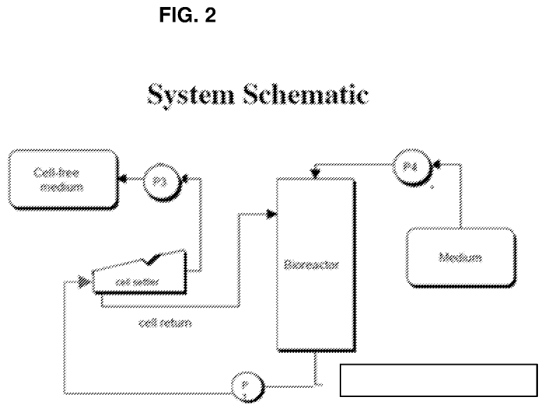

After several iterations of development, a set of 4× glass tube gravity settlers were made. The gravity settler consisted of a bubble-trap and a settling zone joined at a 45° angle. The total holdup volume was approximately 40 mL. This design showed that it could achieve a biomass retention efficiency of >60% when the following criteria were met:
Cell sedimentation rate was >0.03 mm/min
Fermentation feed-rate was <0.15 ml/min.
A dip-tube with an opening of ˜0.4 mm was located at the bottom and middle of the vessel. This minimized the number of bubbles entering the system.
A re-circulation flow-rate of approximately 2.5 ml/min was used. This helped to minimze number of bubbles entering the system.
The bubble trap was periodically refilled with whole cell broth (WCB) using a pinch valve-timer module (FIG. 3B and FIG. 3C) to help purge bubbles and prevent them from accumulating and entering the settling zone.
   One normally-open (NO) pinch valve was used for the return line connected to the vessel. This valve was closed during the purge cycle.
   One normally-closed (NC) pinch valve was used for the bubble trap vent and connected to the effluent bottle. This valve was open during the purge cycle.
   Cycle timer was set to 1 hour
   Purge timer was set to 20 seconds
      These resulted in the glass tube gravity settler shown FIG. 2 along with the design parameters in identified in Table 1.

TABLE 1
Design parameteres of the glass tube gravity settler

| | | | Settler | Settler |
|---|---|---|---|---|
| Bubble Trap | Bubble Trap | Bubble Trap | Zone | Zone |
| Diameter | Height | Location | Diameter | Height |
| 1.0 cm | 10.0 cm | 1.0 cm from bottom | 1.0 cm | 30.0 cm |
| | | of Settler Zone | | |

In an initial experiment, a well-growing strain designated Y21900 was used. It grew very well and easily consumed 110 mmol/L/hr of oxygen (OUR) within 24 hours after inoculation and maintained that OUR until the harvest. Consequently, cell retention using the gravity settler did not increase the overall oxygen consumption of the culture and there was not a significant impact on the volumetric productivity. Still, higher biomass concentrations were attained, lower total biomass was produced, and a higher farnesene yield was achieved.

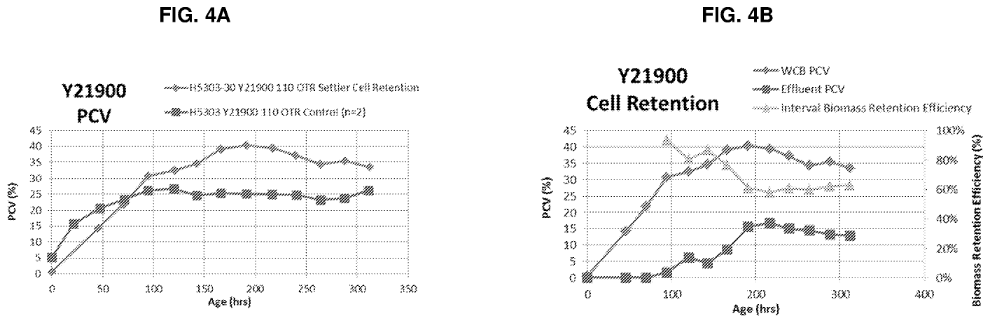

An experiment was then performed where the Y21900 strain was run in a 2 L vessel with an inclined commercially available plate gravity settler. The cell retention process was started after 72 hours, and the settler worked well to retain biomass. The packed cell volume (PCV) of the whole cell broth was 50-60% higher compared to the FAD controls (FIG. 4A), where the FAD control means fill and draw, meaning periodically filling with a given fluid then drawing down, versus simultaneously filling and drawing so the fluid level remains constant.

The biomass retention efficiency was initially ˜90%, but slowly decreased to ˜60% over the subsequent ˜100 hours where it remained stable (FIG. 4B). It should be noted that this experiment used an inoculation ratio of 1% while the ratio standardly used is 30%. This lower initial density that was used caused a slight delay to reach the maximum cell density.

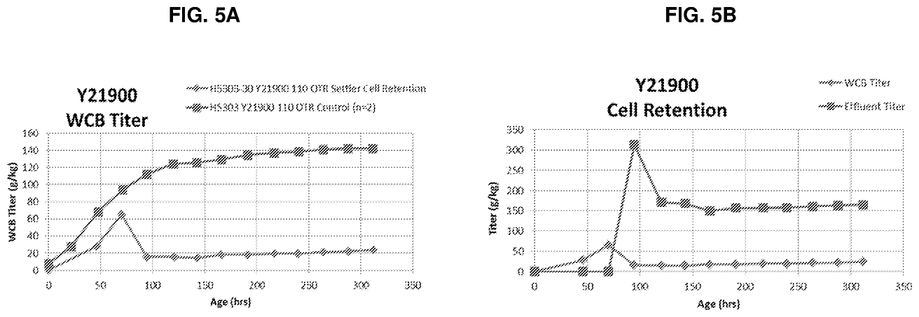

Over the course of this experiment, the inclined plate gravity settler worked well to remove farnesene from the vessel and provided an effluent stream enriched with farnesene (FIG. 5A and FIG. 5B).

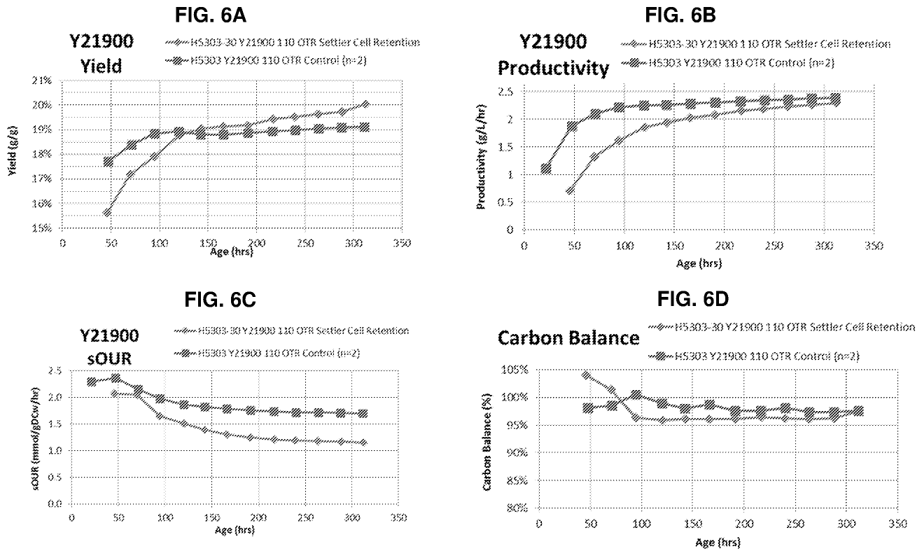

Overall, the retention of biomass in this experiment led to a fermentation yield increase of ˜1.0% absolute, and the volumetric productivity was similar compared to the FAD controls (FIG. 6A and FIG. 6B). The specific oxygen uptake (sOUR) (mmol/L/h/cell) was also lower than the FAD control (FIG. 6C), which aligns with the rate-yield-coupling relationship observed previously. The lag in performance can be attributed to the lower initial cell density. It should also be noted that the contents of the settler were drained back into the vessel at harvest only and thus the performance at harvest was the most accurate time-point. Regardless of these details, the carbon balance was between 95% and 100% for most of the fermentation (FIG. 6D).

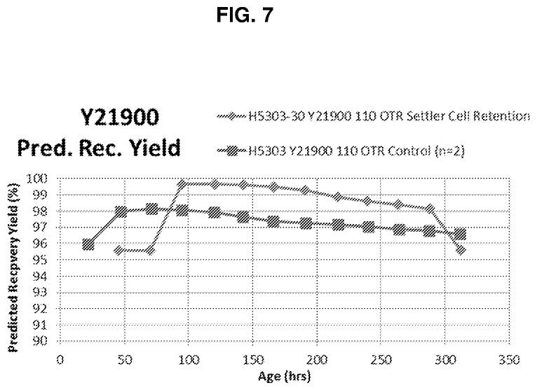

The predicted integrated recovery yield was similar at harvest and was slightly higher during the run (FIG. 7). The higher predicted recovery yield during the run was due to a lower biomass concentration and thus lower farnesene-cell association in the cell retention effluent compared to the whole cell broth draws from the FAD controls. The predicted recovery yield decreased at harvest because the harvest broth had higher concentration of biomass and thus a higher farnesene-cell association in comparison the effluent from earlier in the process.

### Example 2. Continuous Separation of Farnesene Product from Fermentation Using in House Gravity Settling Device

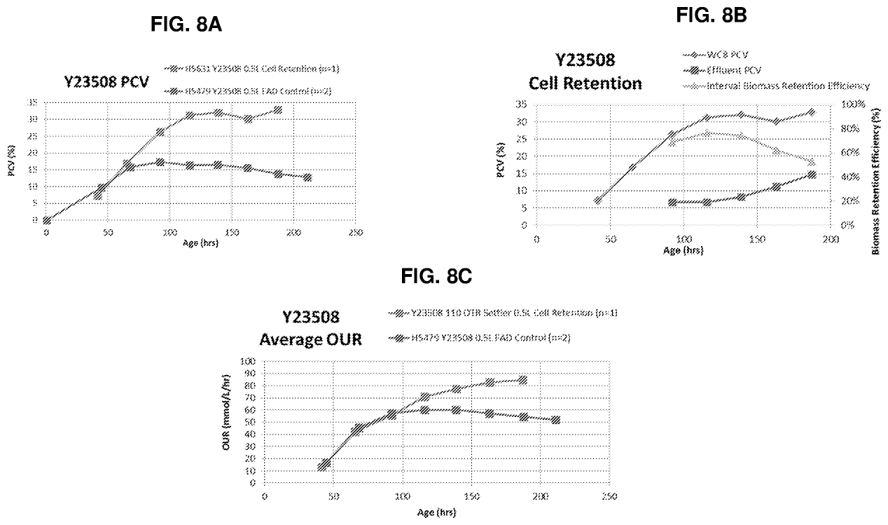

In another experiment, the strain Y23508 was tested. This strain was able to achieve 110 mmol O₂/L/hr during the growth phase, but it was unable to maintain 110 mmol O₂/L/hr during the later stages of the fermentation. The strain appeared to be healthy with very little byproduct accumulation and was thus considered likely to benefit significantly from increasing cell retention. Following these initial Y23508 results, the strain was tested in the cell retention process using an in-house gravity settling device. The fermentation progressed as expected and the cell retention process was started after 72 hours using an inclined tube gravity settler, as described above and in FIG. 3A. The PCV of the whole cell broth was ˜100% higher compared to the FAD controls (FIG. 8A). The biomass retention efficiency was initially ˜75%, but decreased to ˜50% as the culture aged (FIG. 8B). Overall, use of the inclined gravity settling device led to a significant increase in the amount of oxygen consumed (FIG. 8C).

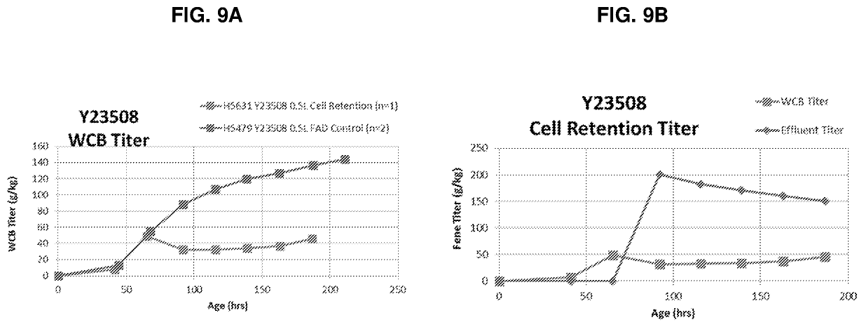

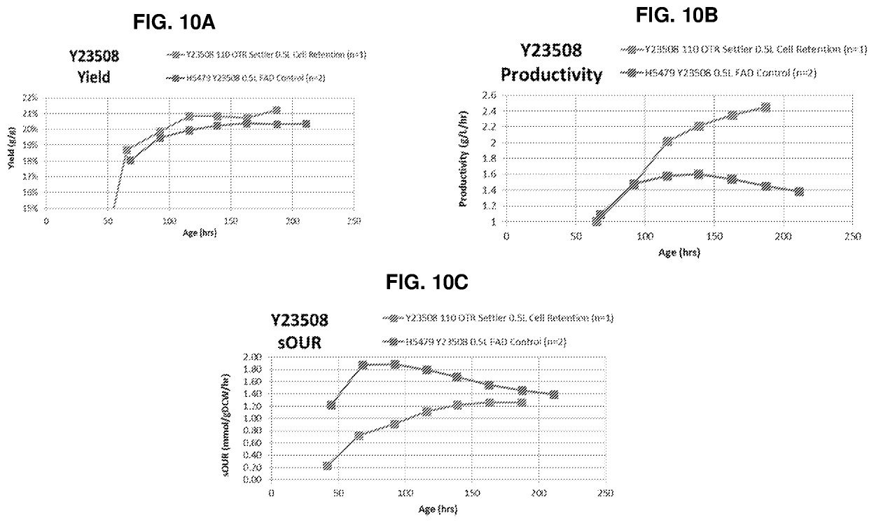

The gravity settler worked well to remove farnesene from the vessel and enriched the effluent stream. However, the farnesene concentration in the vessel was approximately double that of the Y21900 experiment (FIGS. 9A and 9B). This was likely caused by the fact that the tube settler had a lower separation efficiency compared to the plate settler used previously with 2 L Y21900 run. The increase in cell retention increased the fermentation yield by ˜1.0% absolute (FIG. 10A) and the volumetric productivity by 70% compared to the controls (FIG. 101B). The sOUR was also measured (FIG. 10C).

### Example 3. Continuous Separation of Farnesene Product from Fermentation with Various Sugar Concentrations Using Gravity Settling Device

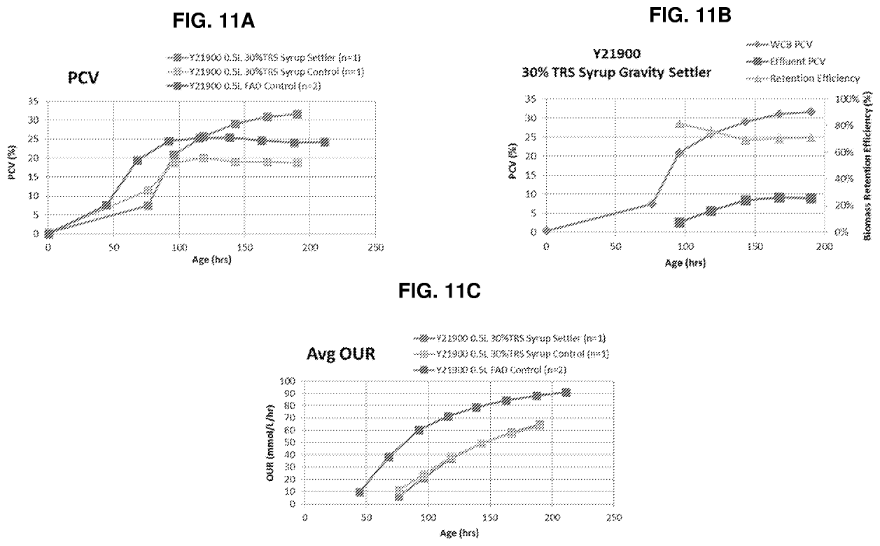

These experiments used cane syrup with a sugar concentration of approximately 60% TRS. However, another source of sugar may be used. The Y21900 strain was used in 0.5 L vessels (e.g., fermentation vessels) with and without a tube settler using dilute cane syrup with a sugar concentration of approximately 30% TRS. A long, 2× longer than the experiments described above, tube settler was used to ensure a high separation efficiency due to the higher dilution rate achieved with the dilute feed. The cell retention process was started after 72 hours, and the gravity settling device worked well to retain biomass. The PCV of the whole cell broth was 50-60% higher compared to the dilute syrup control and ˜30% higher than the concentrated syrup control (FIG. 11A). The biomass retention efficiency was initially ˜80%, but decreased to ˜70% over the subsequent ˜50 hours (FIG. 11B). The OUR for the dilute feed fermentation was significantly lower than the concentrated feed due to the lower sugar concentration (FIG. 11C).

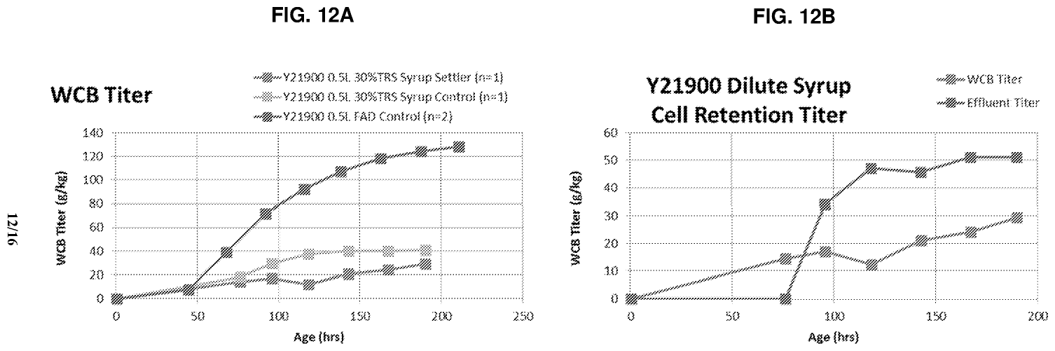

The gravity settler did successfully remove farnesene from the vessel, but not as well as in previous experiments due to the low sugar concentration of the dilute feed (FIG. 12A and FIG. 12B). It is not clear why this separation was lower, but it may be because the distribution of emulsion and farnesene droplets with a dilute feed was different and there were more small droplets that have slower rising velocities.

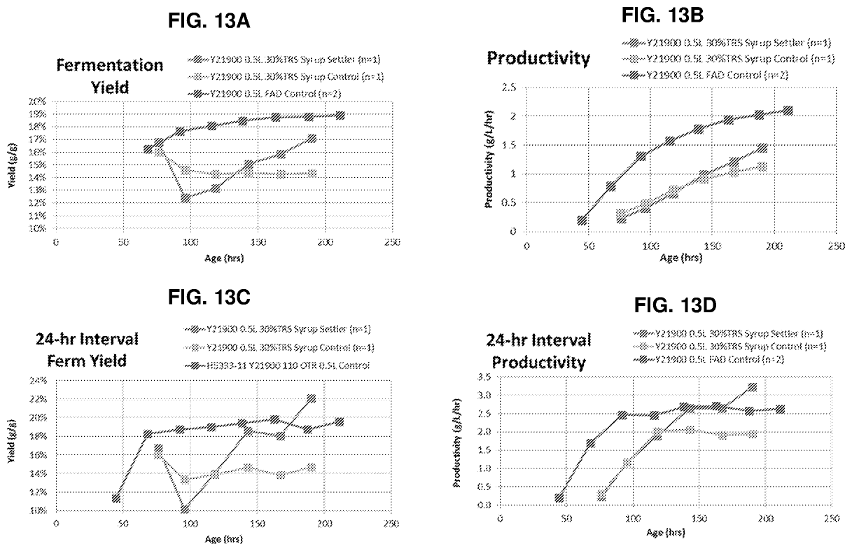

The retention of biomass led to a fermentation yield increase of ˜3.0% absolute (˜20%) and an increase of volumetric productivity of 0.30 g/L/hr (˜25%) compared to the dilute syrup control (FIGS. 13A and 13B). Interestingly, the 24-hr interval yields and productivities for the dilute syrup settler process were similar to the concentrated syrup control at the later time-points (FIG. 13C and FIG. 13D).

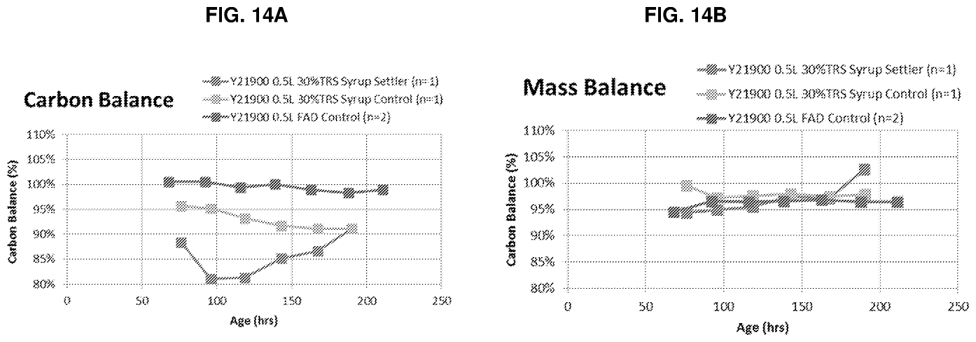

The carbon balance was low during the operation of the cell settler, likely due to the contents (biomass and farnesene) of the settler not being included in the analysis prior to harvest. However, the carbon balance was approximately 90% prior to the start of the settler operation and also at harvest (FIG. 14A). The mass balance was good at 95-103% for the entire run (FIG. 14B).

The designed tube gravity settler, described above, is now available to assess the performance of strains using cell retention. This is a particularly interesting process for strains that are healthy and have a high production yield yet cannot maintain a high enough biomass concentration to consume all of the available oxygen in the vessel. A settler can be used in a continuous mode to increase the biomass concentration during 0.5 L fermentations with >50% retention efficiency while selectively removing farnesene. Due to rate yield coupling, higher steady state biomass concentrations typically result in a 1.0% yield improvement in concentrated cane syrup and can be as high as 3.0% with dilute cane syrup. The impact on volumetric productivity depends on the strain and the sugar concentration of the feedstock. In the case of a well growing strain like Y21900, the effect is minimal. However, in the case of a poor growing strain like Y23508, the effect is significant with a volumetric productivity improvement of approximately 70% primarily due to the increase in cell density and overall oxygen consumption. With low sugar concentration feedstocks, the impact on productivity is not as high, but still significant and can be as high as +25%.

### Example 4. Continuous Separation of Myrcene Product from Fermentation Using Gravity Settling

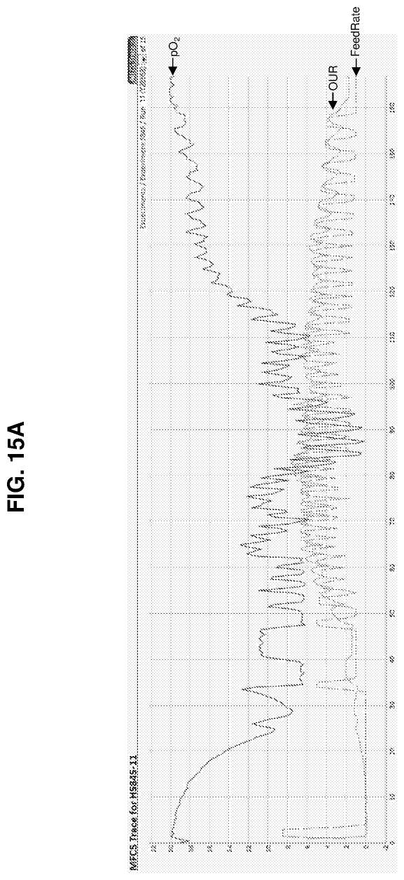

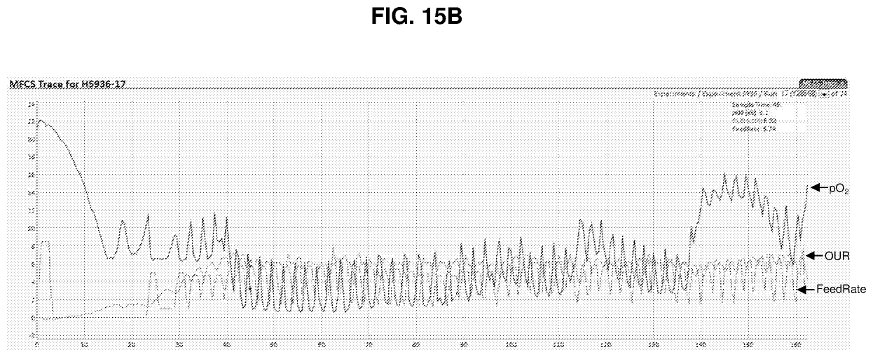

Myrcene, a monoterpene oil, is very inhibitory to host cells (50-100 mg/L IC50) and its presence can cause a variety of problems during fermentations. In a representative fermentation, dissolved oxygen (DO) rose sharply around 90 hr and the oxygen uptake rate (OUR) dropped. Biomass, product titers, and productivity all declined, and mevalonate, a pathway intermediate, rose and accumulated. In this case, increasing the overlay to 25% (of full volume) to sequester the inhibitory myrcene helped extend the time spent in microaerobic conditions but did not fully relieve the problem (FIG. 15A).

A cell settler device as described in Example 1 and illustrated in FIG. 2 and FIG. 3 was used in the context of myrcene production to separate the oil phase from the fermentation whole cell broth and return the cells back to the tank. As the broth descended down into the main tube, the oil phase separated based on density and was pumped out of the top outlet. The aqueous phase and cells returned to the tank via the bottom outlet. With this approach, the oil phase containing the inhibitory myrcene was removed from the tank and collected in an effluent bottle, while the cells were recovered and returned to the tank, thus minimizing loss of biomass.

A total of 3 myrcene strains in 10 total cell settler tanks were tested. Y27992, Y28568, and Y28799 were a ladder of strains, each with 2 additional copies of myrcene synthase than the preceding strain. Y27992 and Y28799 were tested at 60 mmol/L/hr OUR and 10% overlay runs. Y28568 was tested under several different conditions (5%, 10%, and 25% overlay. All runs are summarized below (“Without” and “With” the cell settler) to show the benefit of the cell settler for these three strains. The strains ladder in performance both with and without the cell settler, thus also showing the benefit of increasing copy number of myrcene synthase. In addition, as shown in FIG. 15B, DO and OUR remained stable well beyond 130 hr. of fermentation.

TABLE 2
Yield and productivity at 60 mmol/L/hr
OUR and 10% overlay runs

| | Y27992 | | Y28568 | | Y28799 | |
|---|---|---|---|---|---|---|
| | Without | With | Without | With | Without | With |
| 3-6 Day | 4.4 ± | 5.8 ± | 5.8 ± | 8.5 ± | 8.8 ± | 9.7 |
| yield, wt % | 0.4 | 0.3 | 1.4 | 1.1 | 0.5 | |
| 3-6 Day | 0.158 ± | 0.321 ± | 0.205 ± | 0.371 ± | 0.242 ± | 0.376 |
| productivity, | 0.032 | 0.028 | 0.033 | 0.038 | 0.001 | |
| g/L/hr | | | | | | |

TABLE 3
OUR for strain Y28568 at 5% overlay and 25% overlay

| Median Y28568 | | |
|---|---|---|
| Fermentation | | |
| Average Day 3-7 OUR | Without Cell Settler | With Cell Settler |
| 25% overlay, 0.6 LPM | 44 ± 7 mmol/L/hr | 57 mmol/L/hr (n = 1) |
| | (n = 10) | |
| 5% overlay, 1 LPM | 35 mmol/L/hr (n = 1) | 62 mmol/L/hr (n = 1) |

TABLE 4
Yield for strain Y28568 at 5% overlay and 25% overlay

| Median Y28568 | | |
|---|---|---|
| Fermentation | | |
| Day 3-7 Yield | Without Cell Settler | With Cell Settler |
| 25% overlay, 0.6 LPM | 6.0 ± 1.3% (n = 10) | 7.1% (n = 1) |
| 5% overlay, 1 LPM | 4.5% (n = 1) | 7.0% (n = 1) |

TABLE 5
Productivity for strain Y28568 at 5% overlay and 25% overlay

| Median Y28568 | | |
|---|---|---|
| Fermentation | | |
| Day 3-7 Productivity | Without Cell Settler | With Cell Settler |
| 25% overlay, 0.6 LPM | 0.20 ± 0.03 g/L/hr (n = 10) | 0.34 g/L/hr (n = 1) |
| 5% overlay, 1 LPM | 0.14 g/L/hr (n = 1) | 0.35 g/L/hr (n = 1) |

### Example 5. Continuous Separation of Cell Culture Product from Host Cell Culture Using Gravity Settling

Using conventional techniques or techniques described herein an inclined gravity settler may be used to separate host cells in a cell culture composition from a cell culture product. The host cells may be, for example, bacterial cells. It is important to remove the cell culture product from the cell culture due to the fact that as the concentration of cell culture product in the cell culture composition increases it becomes inhibitory to the cells in the culture. By separating the cell culture product from the bacteria cells, the bacteria cells may be returned to the vessel and cell product may be removed. The bacteria cells may be, for example, host cells from E. coli, B. subtilis, Actinomyces, or Aspergillus.

The cell culture product may be, for example, an isoprenoid or a terpene. The isoprenoid may be a C₅-C₄₀ isoprenoid (e.g., a C₅ isoprenoid, C₁₀ isoprenoid, C₁₅ isoprenoid, C₂₀ isoprenoid, C₂₅ isoprenoid, C₃₀ isoprenoid, C₃₅ isoprenoid, or C₄₀ isoprenoid). For example, in some embodiments, the isoprenoid is a C₂₀ isoprenoid. These compounds are derived from four isoprene units and also called diterpenoids. Illustrative examples of diterpenoids are casbene, eleutherobin, paclitaxel, prostratin, pseudopterosin, and taxadiene. The isoprenoid may be a C₂₀₊ isoprenoid. These compounds are derived from more than four isoprene units and include: triterpenoids (C₃₀ isoprenoid compounds derived from 6 isoprene units) such as arbrusidee, bruceantin, testosterone, progesterone, cortisone, digitoxin, and squalene; tetraterpenoids (C₄₀ isoprenoid compounds derived from 8 isoprenoids) such as P-carotene; and polyterpenoids (C₄₀₊ isoprenoid compounds derived from more than 8 isoprene units) such as polyisoprene. In other embodiments, the isoprenoid is a C15 isoprenoid. These compounds are derived from three isoprene units and are also called sesquiterpenoids. Illustrative examples of sesquiterpenoids are periplanone B, gingkolide B, amorphadiene, artemisinin, artemisinic acid, valencene, nootkatone, epicedrol, epiaristolochene, farnesol, gossypol, sanonin, periplanone, forskolin, and patchoulol, which is also known as patchouli alcohol. In some embodiments, the isoprenoid is selected from the group consisting of abietadiene, amorphadiene, carene, a-farnesene, P-farnesene, farnesol, geraniol, geranylgeraniol, isoprene, linalool, limonene, myrcene, nerolidol, ocimene, patchoulol, P-pinene, sabinene, y-terpinene, terpinolene, valencene, retinol, phytol, retinal, santalol, santalene, sinensol, squalene, bisabolol, and sclareol. The isoprenoid cell culture product may be a C₅-C₂₀ isoprenoid (e.g., C₅ isoprenoid, C₆ isoprenoid, C₇ isoprenoid, C₈ isoprenoid, C₉ isoprenoid, C₁₀ isoprenoid, C₁₁ isoprenoid, C₁₂ isoprenoid, C₁₃ isoprenoid, C₁₄ isoprenoid, C₁₅ isoprenoid, C₁₆ isoprenoid, C₁₇ isoprenoid, C₁₈ isoprenoid, C₁₉ isoprenoid, or C₂₀ isoprenoid). In some embodiments, the isoprenoid produced by the cell is a C₅ isoprenoid. These compounds are derived from one isoprene unit and are also called hemiterpenoids. An illustrative example of a hemiterpenoid is isoprene. The isoprenoid cell culture product may be a C₁₀-C₁₅ isoprenoid (e.g., C₁₀ isoprenoid, C₁₁ isoprenoid, C₁₂ isoprenoid, C₁₃ isoprenoid, C₁₄ isoprenoid, or C₁₅ isoprenoid). In other embodiments, the isoprenoid is a C₁₀ isoprenoid. These compounds are derived from two isoprene units and are also called monoterpenoids. Illustrative examples of monoterpenoids are limonene, citranellol, geraniol, menthol, perillyl alcohol, linalool, thujone, and myrcene. In other embodiments, the isoprenoid is a C₁₅ isoprenoid. These compounds are derived from three isoprene units and are also called sesquiterpenoids. Illustrative examples of sesquiterpenoids are periplanone B, gingkolide B, amorphadiene, artemisinin, artemisinic acid, valencene, nootkatone, epicedrol, epiaristolochene, farnesol, gossypol, sanonin, periplanone, forskolin, and patchoulol, which is also known as patchouli alcohol. Isoprenoid compounds also include, but are not limited to, carotenoids (such as lycopene, a- and P-carotene, a- and P-cryptoxanthin, bixin, zeaxanthin, astaxanthin, and lutein), steroid compounds, cannabinoids, and compounds that are composed of isoprenoids modified by other chemical groups, such as mixed terpene-alkaloids, and coenzyme Q-10. The isoprenoid may be a hemiterpenoid, monoterpenoid, sesquiterpenoid, diterpenoid, sesterterpenoid, triterpenoid, tetraterpenoid, or polyterpenoid. In some embodiments, the isoprenoid is a monoterpenoid.

The cell culture product may be a terpene. The terpene may be a C₅-C₄₀ terpene (e.g., a C₅ terpene, C₁₀ terpene, C₁₅ terpene, C₂₀ terpene, C₂₅ terpene, C₃₀ terpene, C₃₅ terpene, or C₄₀ terpene). In some embodiments, the terpene is a C₅-C₂₀ terpene (e.g., C₅ terpene, C₆ terpene, C₇ terpene, C₈ terpene, C₉ terpene, C₁₀ terpene, C terpene, C₁₂ terpene, C₁₃ terpene, C₁₄ terpene, C₁₅ terpene, C₁₆ terpene, C₁₇ terpene, C₁₈ terpene, C₁₉ terpene, or C₂₀ terpene). The terpene may be a C₁₀-C₁₅ terpene (e.g., C₁₀ terpene, C terpene, C₁₂ terpene, C₁₃ terpene, C₁₄ terpene, or C₁₅ terpene). The terpene may be hemiterpene, monoterpene, sesquiterpene, diterpene, sesterterpene, triterpene, tetraterpene, or polyterpene.

According to conventional methods or the methods disclosed herein, a cell culture product may be produced by culturing, in a vessel, a population of bacteria cells capable of producing the cell culture product in an aqueous-phase culture medium and under conditions suitable for the bacterial host cells to produce the cell culture product, thereby producing the cell culture product and forming a cell culture composition. Any conventional method for culturing bacterial cells may be used. The cell culture product may be produced by culturing bacterial host cells capable of synthesizing the cell culture product in a vessel. The vessel may have a capacity of between 1,000,000 L and 0.5 L; for example, the vessel may have a capacity of between 0.5 L and 500,000 L, 0.5 L and 100,000 L, or 0.5 L and 1,000 L.

The aqueous phase containing the bacteria cells may be separated from the oil-emulsion phase containing the cell culture product using gravity separation. The gravity separation process may include cell sedimentation. The gravity separation may be achieved using a gravity settling device. The gravity settling device may include an inlet tube that is in fluid communication with, and that receives the portion of the mixture from, the vessel. The gravity settling device may include a settling chamber that is in fluid communication with, and that receives the portion of the mixture from, the inlet tube. The settling chamber may have an incline angle of greater than 0° and less than, or equal to, 90°. For example, the settling chamber may have an incline angle of from about 25° to about 75°. The settling chamber may have an incline angle of about 45°. The gravity settling device may include an outlet at the bottom of the settling chamber that is in fluid communication with the vessel. The gravity settling device may include an outlet at the top of the settling chamber that is in fluid communication with an effluent vessel. The gravity settling device includes an overflow outlet at the top of the inlet tube that is in fluid communication with the effluent vessel. The overflow may be returned to the effluent vessel.

The cell sedimentation process may include introducing the portion of the mixture including the cell culture composition into the inlet tube. The plurality of the bacterial host cells flow to the bottom of the settling chamber. These host cells may then return to the vessel through the outlet at the bottom of the settling chamber. The water-immiscible solvent may be removed from the settling chamber through the outlet at the top of the settling chamber and the water-immiscible solvent is delivered to the effluent bottle. Any excess mixture that exceeds the volume of the settling chamber may be removed through the overflow outlet and delivered the to the effluent vessel. The host cells may be sedimented at a rate of about 0.03 mm/min or greater. For example, the host cells are sedimented at a rate of from about 0.003 mm/min or greater, optionally wherein the host cells are sedimented at a rate of from about 0.003 mm/min to about 0.5 mm/min (e.g., about 0.003 mm/min to about 0.4 mm/min, about 0.003 mm/min to about 0.3 mm/min, about 0.003 mm/min to about 0.2 mm/min, about 0.003 mm/min to about 0.1 mm/min, about 0.003 mm/min to about 0.05 mm/min, about 0.003 mm/min to about 0.01 mm/min, about 0.003 mm/min to about 0.005 mm/min, about 0.005 mm/min to about 0.5 mm/min, about 0.01 mm/min to about 0.5 mm/min, about 0.05 mm/min to about 0.5 mm/min, about 0.1 mm/min to about 0.5 mm/min about 0.2 mm/min to about 0.5 mm/min, about 0.3 mm/min to about 0.5 mm/min, or about 0.4 mm/min to about 0.5 mm/min). In some embodiments, the plurality of the host cells is returned to the vessel at a rate of from about 1 ml/L/min to about 300 ml/L/min (e.g., about 1 ml/L/min to about 250 ml/L/min, about 1 ml/L/min to about 200 ml/L/min, about 1 ml/L/min to about 150 ml/L/min, about 1 ml/L/min to about 100 ml/L/min, about 1 ml/L/min to about 50 ml/L/min, about 1 ml/L/min to about 25 ml/L/min, about 2 ml/L/min to about 300 ml/L/min, about 3 ml/L/min to about 300 ml/L/min, about 4 ml/L/min to about 300 ml/L/min, about 10 mL/L/min to about 300 mL/L/min, about 50 mL/L/min to about 300 mL/L/min, about 100 mL/L/min to about 300 mL/L/min, about 150 mL/L/min to about 300 mL/L/min, about 200 mL/L/min to about 300 mL/L/min, or about 250 mL/L/min to about 300 mL/L/min).

A pump may be used in the gravity settling device. In some embodiments, the portion of the mixture including the cell culture composition is delivered to the inlet tube by way of a pump. In some embodiments, the water-immiscible solvent is removed from the settling chamber and delivered through the outlet at the top of the settling chamber to the effluent bottle by way of a pump.

Pinch valves may be used in to control the amount of cell culture composition entering the cell settling device. These pinch valves may be controlled by timers, e.g., as shown in FIG. 3B and FIG. 3C. In some embodiments, the opening and closing of pinch valves may be automated by monitoring the volume of liquid in the bubble trap. The volume of liquid in the bubble trap may be monitored, for example, using a float switch, a light curtain sensor, or sensors for monitoring capacitance.

The aqueous phase of the cell culture composition may be oxygenated. Oxygenating of the aqueous phase of the cell culture composition may be by pumping air into the vessel. The air may be compressed air. The compressed air may be delivered by pressure.

The aqueous phase of the cell culture composition may be mixed using an impeller. Furthermore, the aqueous phase of the cell culture composition and the water-immiscible solvent may be mixed using an impeller.

Producing the cell culture product in a cell culture composition may include contacting the cell culture composition with a water-immiscible solvent. The cell culture product may be a water-immiscible compound. The water-immiscible solvent may be added to the cell culture composition to a final concentration of water-immiscible solvent of from about 0.5% (v/v) to about 50% (v/v) e.g., about 0.5% (v/v) to about 10% (v/v), about 0.5% (v/v) to about 20% (v/v), about 0.5% (v/v) to about 30% (v/v), about 0.5% to about 40% (v/v), about 40% (v/v) to about 50% (v/v), about 30% (v/v) to about 50% (v/v), about 20% (v/v) to about 50% (v/v), about 10% (v/v) to about 50% (v/v), or about 1% (v/v) to about 5% (v/v)). The water-immiscible solvent may be added to the cell culture composition to a final concentration of water-immiscible solvent of from about 5% (v/v) to about 25% (v/v). The water-immiscible may have a log(K_{d}) value of from about 1 to about 15, where K_{d} is the partition coefficient for the cell culture product between the water-immiscible solvent and the cell culture composition. For example, the log (K_{d}) may be about 1.5, about 1.6, about 1.7, about 1.8, about 1.9, about 2, about 2.1, about 2.2, about 2.3, about 2.4, about 2.5, about 2.6, about 2.7, about 2.8, about 2.9, or about 3. In some embodiments, the water-immiscible solvent may log(K_{d}) value of from about 2 to about 3. For example, the water-immiscible solvent has a log(K_{d}) value of about 2, about 2.1, about 2.2, about 2.3, about 2.4, about 2.5, about 2.6, about 2.7, about 2.8, about 2.9, or about 3. The cell culture product may have a log(D) value of from about 1 to about 15 (e.g., about 1.1, 1.2, 1.3, 1.4, 1.5, 1.6, 1.7, 1.8, 1.9, 2, 2.1, 2.2, 2.3, 2.4, 2.5, 2.6, 2.7 2.8, 2.9, 3.1, 3.2, 3.3, 3.4, 3.5, 3.6, 3.7, 3.8, 3.9, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.22, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, or 15). The water-immiscible solvent may be an alcohol. For example, the water-immiscible solvent may be a C₁₀-C₂₀ alcohol (e.g., C₁₀ alcohol, C₁₁ alcohol, C₁₂ alcohol, C₁₃ alcohol, C₁₄ alcohol, C₁₅ alcohol, C₁₆ alcohol, C₁₇ alcohol, C₁₈ alcohol, C₁₉ alcohol, or C₂₀ alcohol). The water-immiscible solvent may be a C₁₂-C₁₈ alcohol (e.g., C₁₂ alcohol, C₁₃ alcohol, C₁₄ alcohol, C₁₅ alcohol, C₁₆ alcohol, C₁₇ alcohol, or C₁₈ alcohol). In some embodiments, the water-immiscible solvent is a vegetable oil. In some embodiments, the water-immiscible solvent is corn oil, sunflower oil, soybean oil, mineral oil, polyalphaolefin, dodecane, hexadecane, oleyl alcohol, butyl oleate, dibutyl phthalate, dodecanol, dioctyl phthalate, farnesene, or isopropyl myristate. The water-immiscible solvent may be a Drakeol™ fluid. The water-immiscible solvent may be, for example, a Jarcol™ fluid or a Durasyn™ fluid.

The cell culture product may be isolated from the cell culture composition using a filter, optionally wherein the filter is located in the vessel. The filter may separate the water-immiscible solvent from the cell culture composition in the vessel. The filter may capable of separating at least a portion of the cell culture product from at least a portion of the host cells in the vessel.

The resulting cell culture product may recovered using these processes and methods. The bacterial host cells from the cell culture composition may then be returned to the vessel for continued culture.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of producing a cell culture product, the method comprising:
(a) culturing, in a vessel, a population of host cells capable of producing the cell culture product in an aqueous-phase culture medium and under conditions suitable for the host cells to produce the cell culture product, thereby producing the cell culture product and forming a cell culture composition;
(b) contacting the cell culture composition with a water-immiscible solvent, thereby (i) forming a mixture and (ii) partitioning the cell culture product between the cell culture composition and the water-immiscible solvent;
(c) selecting a portion of the mixture resulting from (b);
(d) from the portion of the mixture selected in (c), separating a plurality of the host cells from the cell culture composition;
(e) returning the plurality of the host cells to the vessel; and
(f) from the portion of the mixture selected in (c), recovering the cell culture product from the water-immiscible solvent.

2. A method of isolating a cell culture product from a cell culture composition, the method comprising:
(a) providing, in a vessel, a cell culture composition that has been produced by culturing a population of host cells capable of producing the cell culture product in an aqueous-phase culture medium and under conditions suitable for the host cells to produce the cell culture product;
(b) contacting the cell culture composition with a water-immiscible solvent, thereby (i) forming a mixture and (ii) partitioning the cell culture product between the cell culture composition and the water-immiscible solvent;
(c) selecting a portion of the mixture resulting from (b);
(d) from the portion of the mixture selected in (c), separating a plurality of the host cells from the cell culture composition;
(e) returning the plurality of the host cells to the vessel; and
(f) recovering the cell culture product from the water-immiscible solvent resulting from (c).

3. A method of isolating a cell culture product from a cell culture composition, the method comprising:
(a) providing, in a vessel, a mixture comprising (i) a water-immiscible solvent and (ii) a cell culture composition that has been produced by culturing a population of host cells capable of producing the cell culture product in an aqueous-phase culture medium and under conditions suitable for the host cells to produce the cell culture product, wherein the cell culture product is partitioned between the cell culture composition and the water-immiscible solvent;
(b) selecting a portion of the mixture;
(c) from the portion of the mixture selected in (b), separating a plurality of the host cells from the cell culture composition;
(d) returning the plurality of the host cells to the vessel; and
(e) recovering the cell culture product from the water-immiscible solvent resulting from (c).

4. The method of claim 1, the method comprising repeating steps (a)-(f) a plurality of times, optionally wherein the method comprises repeating steps (a)-(f) continuously or discontinuously.

5. The method of claim 2, the method comprising repeating steps (b)-(f) a plurality of times, optionally wherein the method comprises repeating steps (b)-(f) continuously or discontinuously.

6. The method of claim 3, the method comprising repeating steps (b)-(e) a plurality of times, optionally wherein the method comprises repeating steps (b)-(e) continuously or discontinuously.

7. The method of any one of claims 1-6, wherein the method further comprises adding fresh water-immiscible solvent to the cell culture composition following the selecting step.

8. The method of any one of claims 1-7, wherein the plurality of the host cells is separated from the water-immiscible solvent by way of a gravity separation process.

9. A method of producing a cell culture product, the method comprising:
(a) culturing, in a vessel, a population of host cells capable of producing the cell culture product in an aqueous-phase culture medium and under conditions suitable for the host cells to produce the cell culture product, thereby producing the cell culture product and forming a cell culture composition;
(b) selecting a portion of the cell culture composition;
(c) separating a plurality of the host cells from the portion of the cell culture composition selected in (b);
(d) returning the plurality of the host cells to the vessel; and
(e) recovering the cell culture product from the portion of the cell culture composition selected in (b), wherein the cell culture product comprises:
i) a compound that is water-immiscible, optionally a compound having a log(D) value of from about 1 to about 15,
ii) a compound that is inhibitory to the host cells, and/or
iii) an isoprenoid or terpene.

10. The method of claim 9, wherein the plurality of the host cells is separated from the cell culture composition by way of a gravity separation process.

11. The method of claim 8 or 10, wherein the gravity separation process comprises cell sedimentation.

12. The method of claim 11, wherein the cell sedimentation is achieved using a gravity settling device.

13. The method of claim 12, wherein the gravity settling device comprises:
(i) an inlet tube that is in fluid communication with, and that receives the portion of the mixture from, the vessel;
(ii) a settling chamber that is in fluid communication with, and that receives the portion of the mixture from, the inlet tube, wherein the settling chamber has an incline angle of greater than 0° and less than, or equal to, 90°, optionally wherein the settling chamber has an incline angle of from about 25° to about 75°, optionally wherein the settling chamber has an incline angle of from about 35° to about 55°, optionally wherein the settling chamber has an incline angle of about 45°;
(iii) an outlet at the bottom of the settling chamber that is in fluid communication with the vessel;
(iv) an outlet at the top of the settling chamber that is in fluid communication with an effluent vessel; and, optionally,
(v) an overflow outlet at the top of the inlet tube that is in fluid communication with the effluent vessel, whereby upon introduction into the inlet tube of an excess of the mixture that exceeds the volume of the settling chamber, the excess mixture flows through the overflow outlet and into the effluent vessel.

14. The method of claim 13, wherein the cell sedimentation comprises:
(i) introducing the portion of the mixture into the inlet tube;
(ii) allowing the plurality of the host cells to flow to the bottom of the settling chamber and, subsequently, to return to the vessel through the outlet at the bottom of the settling chamber;
(iii) removing the water-immiscible solvent from the settling chamber through the outlet at the top of the settling chamber and delivering the water-immiscible solvent to the effluent bottle; and, optionally,
(iv) removing any excess mixture that exceeds the volume of the settling chamber through the overflow outlet and delivering the excess mixture to the effluent vessel.

15. The method of claim 13 or 14, wherein the gravity settling device further comprises a bubble trap.

16. The method of claim 15, wherein the bubble trap and the settling chamber are joined at an angle of between about 60° and 120°.

17. The method of claim 16, wherein the bubble trap and the settling chamber are joined at an angle of about 90°.

18. The method of any one of claims 13-17, wherein the portion of the mixture is delivered to the inlet tube by way of a pump.

19. The method of any one of claims 13-18, wherein the water-immiscible solvent is removed from the settling chamber and delivered through the outlet at the top of the settling chamber to the effluent bottle by way of a pump.

20. The method of any one of claims 1-19, wherein the method comprises introducing a carbon source into the vessel.

21. The method of claim 20, wherein the carbon source is continuously introduced into the vessel.

22. The method of claim 20 or 21, wherein the carbon source is introduced into the vessel by way of a pump.

23. The method of any one of claims 1-22, the method comprising oxygenating the cell culture composition.

24. The method of claim 23, wherein the cell culture composition is oxygenated by way of delivering compressed air into the vessel.

25. The method of any one of claims 1-24, the method comprising mixing the cell culture composition by way of an impeller.

26. The method of any one of claims 1-25, wherein the cell culture product is a water-immiscible compound.

27. The method of any one of claims 1-26, wherein the cell culture product is a compound that is inhibitory to the host cells.

28. The method of any one of claims 1-27, wherein the cell culture product is a terpene.

29. The method of claim 28, wherein the terpene is a C₅-C₄₀ terpene.

30. The method of claim 29, wherein the terpene is a C₅-C₂₀ terpene.

31. The method of claim 30, wherein the terpene is a C₁₀-C₁₅ terpene.

32. The method of claim 28, wherein the terpene is a hemiterpene, monoterpene, sesquiterpene, diterpene, sesterterpene, triterpene, tetraterpene, or polyterpene.

33. The method of claim 32, wherein the terpene is a monoterpene.

34. The method of any one of claims 1-27, wherein the cell culture product is an isoprenoid.

35. The method of claim 34, wherein the isoprenoid is a C₅-C₄₀ isoprenoid.

36. The method of claim 35, wherein the isoprenoid is a C₅-C₂₀ isoprenoid.

37. The method of claim 36, wherein the isoprenoid is a C₁₀-C₁₅ isoprenoid.

38. The method of claim 34, wherein the isoprenoid is a hemiterpenoid, monoterpenoid, sesquiterpenoid, diterpenoid, sesterterpenoid, triterpenoid, tetraterpenoid, or polyterpenoid.

39. The method of claim 38, wherein the isoprenoid is a monoterpenoid.

40. The method of any one of claims 1-27, wherein the cell culture product is abietadiene, anethole, amorphadiene, carene, carvacrol, creosol, cuminaldehyde, eugenol, α-farnesene, β-farnesene, farnesol, geranial, geraniol, geranylgeraniol, hinokitiol, isoprene, isoprenol, linalool, limonene, myrcene, nerolidol, ocimene, patchoulol, perillyl alcohol, β-pinene, sabinene, γ-terpinene, terpinolene, menthol, neral, nerol, eucalyptol, citronellol, citronellal, valencene, or a salvinorin.

41. The method of claim 40, wherein the cell culture product is β-farnesene.

42. The method of claim 40, wherein the cell culture product is myrcene.

43. The method of claim 40, wherein the cell culture product is pinene.

44. The method of claim 40, wherein the cell culture product is limonene.

45. The method of claim 40, wherein the cell culture product is menthol.

46. The method of claim 40, wherein the cell culture product is citronellal.

47. The method of claim 40, wherein the cell culture product is citronellol.

48. The method of claim 40, wherein the cell culture product is farnesol.

49. The method of claim 40, wherein the cell culture product is terpinene.

50. The method of claim 40, wherein the cell culture product is terpinolene.

51. The method of claim 40, wherein the cell culture product is geraniol.

52. The method of claim 40, wherein the cell culture product is linalool.

53. The method of any one of claims 1-52, wherein the cell culture product has a minimum inhibitory concentration (MIC) in the host cells of from about 0.1 mM to about 5 mM.

54. The method of claim 53, wherein the cell culture product has a MIC in the host cells of from about 0.1 mM to about 2.5 mM, optionally wherein the cell culture product has a MIC In the host cells of 0.1 mM, about 0.15 mM, about 0.2 mM, about 0.25 mM, about 0.3 mM, about 0.35 mM, about 0.4 mM, about 0.45 mM, about 0.5 mM, about 0.55 mM, about 0.6 mM, about 0.65 mM, about 0.7 mM, about 0.75 mM, about 0.8 mM, about 0.85 mM, about 0.9 mM, about 0.95 mM, about 1 mM, about 1.25 mM, about 1.3 mM, about 1.35 mM, about 1.4 mM, about 1.45 mM, about 1.5 mM, about 1.55 mM, about 1.6 mM, about 1.65 mM, about 1.7 mM, about 1.75 mM, about 1.8 mM, about 1.85 mM, about 1.9 mM, about 1.95 mM, about 2 mM, about 2.25 mM, about 2.3 mM, about 2.35 mM, about 2.4 mM, about 2.45 mM, or about 2.5 mM.

55. The method of any one of claims 1-8 and 11-54, wherein the water-immiscible solvent has a log(K_{d}) value of from about 1 to about 15, wherein the K_{d} is the partition coefficient for the cell culture product between the water-immiscible solvent and the cell culture composition.

56. The method of claim 55, wherein the water-immiscible solvent has a log(K_{d}) value of from about 2 to about 3, optionally wherein the water-immiscible solvent has a log(K_{d}) value of about 2, about 2.1, about 2.2, about 2.3, about 2.4, about 2.5, about 2.6, about 2.7, about 2.8, about 2.9, or about 3.

57. The method of any one of claims 1-56, wherein the cell culture product has a log(D) value of from about 1 to about 15.

58. The method of any one of claims 1-8 and 11-57, wherein the water-immiscible solvent is an alcohol.

59. The method of claim 58, wherein the water-immiscible solvent is a C₁₀-C₂₀ alcohol.

60. The method of claim 59, wherein the water-immiscible solvent is a C₁₂-C₁₈ alcohol.

61. The method of any one of claims 1-8 and 11-57, wherein the water-immiscible solvent is corn oil, sunflower oil, soybean oil, mineral oil, polyalphaolefin, dodecane, hexadecane, oleyl alcohol, butyl oleate, dibutyl phthalate, dodecanol, dioctyl phthalate, farnesene, or isopropyl myristate

62. The method of any one of claims 1-61, wherein the vessel has a capacity of from about 0.5 L to about 600,000 L.

63. The method of claim 62, wherein the vessel has a capacity of from about 0.5 L to about 1,000 L.

64. The method of any one of claims 20-63, wherein the carbon source is introduced into the vessel at a rate of from about 0.1 g TRS/L/hour to about 30 g TRS/L/hour.

65. The method of claim 64, wherein the carbon source is introduced into the vessel at a rate of from about 1 g TRS/L/hour to about 15 g TRS/L/hour.

66. The method of claim 64, wherein the carbon source is introduced into the vessel at a rate of less than 0.15 g TRS/L/hour.

67. The method of claim 66, wherein the carbon source is introduced into the vessel at a rate of from about 0.01 g TRS/L/hour to about 0.15 g TRS/L/hour.

68. The method of any one of claims 20-67, wherein the carbon source introduced into the vessel has a concentration of from about 10% (w/v) to about 80% (w/v) of total reducing sugar.

69. The method of claim 68, wherein the carbon source introduced into the vessel has a concentration of about 30% (w/v) of total reducing sugar.

70. The method of claim 68, wherein the carbon source has introduced into the vessel has a concentration of about 60% (w/v) of total reducing sugar.

71. The method of any one of claims 1-70, wherein the host cells in the cell culture composition consume oxygen at a rate of from about 25 mmol/L/hr to about 250 mmol/L/hr.

72. The method of claim 71, wherein the host cells in the cell culture composition consume oxygen at a rate of from about 90 mmol/L/hr to about 130 mmol/L/hr.

73. The method of claim 72, wherein the host cells in the cell culture composition consume oxygen at a rate of from about 110 mmol/L/hr.

74. The method of any one of claims 11-73, wherein the host cells are sedimented at a rate of about 0.003 mm/min or greater, optionally wherein the host cells are sedimented at a rate of from about 0.0003 mm/min to about 0.5 mm/min.

75. The method of any one of claims 1-74, wherein the plurality of the host cells is returned to the vessel at a rate of from about 1 ml/L/min to about 300 ml/L/min.

76. The method of any one of claims 1-8 and 11-75, wherein the water-immiscible solvent is added to the cell culture composition to a final concentration of water-immiscible solvent of from about 0.5% (v/v) to about 50% (v/v).

77. The method of claim 76, wherein the water-immiscible solvent is added to the cell culture composition to a final concentration of water-immiscible solvent of from about 5% (v/v) to about 25% (v/v).

78. The method of any one of claims 1-77, wherein the host cells comprise one or more heterologous nucleic acids that each, independently, encode an enzyme of the 1-deoxy-D-xylulose 5-diphosphate (DXP) biosynthetic pathway.

79. The method of claim 78, wherein the one or more heterologous nucleic acids encode one or more of a 1-deoxy-D-xylulose-5-phosphate synthase, a 1-deoxy-D-xylulose-5-phosphate reductoisomerase, a 4-diphosphocytidyl-2C-methyl-D-erythritol synthase, 4-diphosphocytidyl-2C-methyl-D-erythritol kinase, a 2C-methyl-D-erythritol 2,4-cyclodiphosphate synthase, and/or 1-hydroxy-2-mehtyl-2-(E)-butenyl-4-diphosphate synthase.

80. The method of claim 78 or 79, wherein the one or more heterologous nucleic acids encoding one or more enzymes of the DXP biosynthetic pathway are integrated into the genome of the host cell.

81. The method of claim 78 or 79, wherein the one or more heterologous nucleic acids encoding one or more enzymes of the DXP biosynthetic pathway are present within one or more plasmids.

82. The method of any one of claims 1-81, wherein the host cells comprise one or more heterologous nucleic acids that each, independently, encode an enzyme of the mevalonate (MEV) biosynthetic pathway.

83. The method of claim 82, wherein the one or more heterologous nucleic acids encode one or more of an acetyl-CoA thiolase, an HMG-CoA synthase, an HMG-CoA reductase, a mevalonate kinase, a phosphomevalonate kinase, a mevalonate pyrophosphate decarboxylase, and/or an IPP:DMAPP isomerase.

84. The method of claim 82 or 83, wherein the one or more heterologous nucleic acids encoding one or more enzymes of the MEV biosynthetic pathway are integrated into the genome of the host cell.

85. The method of claim 83 or 84, wherein the one or more heterologous nucleic acids encoding one or more enzymes of the MEV biosynthetic pathway are present within one or more plasmids.

86. The method of any one of claims 1-85, wherein the host cells comprise one or more heterologous nucleic acids that each, independently, encode an enzyme of the cannabinoid biosynthetic pathway.

87. The method of claim 86, wherein the one or more heterologous nucleic acids encode one or more of an acetyl-CoA thiolase, an HMG-CoA synthase, an HMG-CoA reductase, a mevalonate kinase, a phosphomevalonate kinase, a mevalonate pyrophosphate decarboxylase, and an IPP:DMAPP isomerase.

88. The method of claim 86 or 87, wherein the one or more heterologous nucleic acids encoding one or more enzymes of the cannabinoid biosynthetic pathway are integrated into the genome of the host cell.

89. The method of claim 86 or 87, wherein the one or more heterologous nucleic acids encoding one or more enzymes of the cannabinoid biosynthetic pathway are present within one or more plasmids.

90. The method of any one of claims 1-89, wherein the host cells are yeast cells.

91. The method of claim 90, wherein the yeast cells are Saccharomyces sp. cells or Kluveromyces sp. cells.

92. The method of claim 91, wherein the yeast cells are Saccharomyces cerevisiae cells.

93. The method of claim 91, wherein the yeast cells are Kluveromyces marxianus cells.

94. The method of any one of claims 1-89, wherein the host cells are bacterial cells.

95. The method of any one of claims 1-89, wherein the host cells are fungal cells.

96. The method of any one of claims 1-95, wherein the method results in biomass retention of at least 60%.

97. The method of any one of claims 1-96, wherein the method results in performance enhancement of the host cells relative to a reference method in which a gravity settling device is not used to effect cell sedimentation.

98. The method of claim 97, wherein the method results in a performance enhancement of the host cells of from about 0.5% to about 500% relative to a reference method in which a gravity settling device is not used to effect cell sedimentation.

99. The method of any one of claims 1-98, wherein the method results in an increase in cell culture yield relative to a reference method in which a gravity settling device is not used to effect cell sedimentation.

100. The method of claim 99, wherein the method results in an increase in cell culture yield of from about 0.5% to about 500% relative to a reference method in which a gravity settling device is not used to effect cell sedimentation.

101. The method of any one of claims 1-100, wherein the method is performed aseptically.

102. The method of any one of claims 1-101, wherein the cell culture product is a fermentation product.

103. The method of any one of claims 1-102, wherein the cell culture composition is a fermentation composition.

104. The method of any one of claims 1-103, wherein the vessel is a fermentation vessel.

105. A composition comprising a cell culture product dissolved in a water-immiscible solvent, wherein the composition is obtained by a method comprising:
(a) culturing, in a vessel, a population of host cells capable of producing the cell culture product in an aqueous-phase culture medium and under conditions suitable for the host cells to produce the cell culture product, thereby producing a cell culture composition;
(b) contacting the cell culture composition with a water-immiscible solvent, thereby (i) forming a mixture and (ii) partitioning the cell culture product between the cell culture composition and the water-immiscible solvent;
(c) selecting from the vessel a portion of the mixture resulting from (b);
(d) from the portion of the mixture selected in (c), separating a plurality of the host cells from the water-immiscible solvent;
(e) returning the plurality of the host cells to the vessel; and
(f) recovering the cell culture product from the water-immiscible solvent resulting from (c).

106. The composition of claim 105, wherein the method comprises repeating steps (a)-(f) a plurality of times, optionally wherein the method comprises repeating steps (a)-(f) continuously.

107. The composition of claim 105 or 106, wherein the method further comprises adding fresh water-immiscible solvent to the cell culture composition following the selecting step.

108. The composition of any one of claims 105-107, wherein the plurality of the host cells is separated from the water-immiscible solvent by way of a gravity separation process.

109. The composition of claim 108, wherein the gravity separation process comprises cell sedimentation.

110. The composition of claim 109, wherein the cell sedimentation is achieved using a gravity settling device.

111. The composition of claim 110, wherein the gravity settling device comprises:
(i) an inlet tube that is in fluid communication with, and that receives the portion of the mixture from, the vessel;
(ii) a settling chamber that is in fluid communication with, and that receives the portion of the mixture from, the inlet tube, wherein the settling chamber has an incline angle of greater than 0° and less than, or equal to, 90°, optionally wherein the settling chamber has an incline angle of from about 25° to about 75°, optionally wherein the settling chamber has an incline angle of from about 35° to about 55°, optionally wherein the settling chamber has an incline angle of about 45°;
(iii) an outlet at the bottom of the settling chamber that is in fluid communication with the vessel;
(iv) an outlet at the top of the settling chamber that is in fluid communication with an effluent vessel; and, optionally,
(v) an overflow outlet at the top of the inlet tube that is in fluid communication with the effluent vessel, whereby upon introduction into the inlet tube of an excess of the mixture that exceeds the volume of the settling chamber, the excess mixture flows through the overflow outlet and into the effluent vessel.

112. The composition of claim 111, wherein the cell sedimentation comprises:
(i) introducing the portion of the mixture into the inlet tube;
(ii) allowing the plurality of the host cells to flow to the bottom of the settling chamber and, subsequently, to return to the vessel through the outlet at the bottom of the settling chamber;
(iii) removing the water-immiscible solvent from the settling chamber through the outlet at the top of the settling chamber and delivering the water-immiscible solvent to the effluent bottle; and, optionally,
(iv) removing any excess mixture that exceeds the volume of the settling chamber through the overflow outlet and delivering the excess mixture to the effluent vessel.

113. The method of claim 111 or 112, wherein the gravity settling device further comprises a bubble trap.

114. The method of claim 113, wherein the bubble trap and the settling chamber are joined at an angle of between about 30° and 60°.

115. The method of claim 114, wherein the bubble trap and the settling chamber are joined at an angle of about 45°.

116. The composition of any one of claims 111-115, wherein the portion of the mixture is delivered to the inlet tube by way of a pump.

117. The composition of any one of claims 111-116, wherein the water-immiscible solvent is removed from the settling chamber and delivered through the outlet at the top of the settling chamber to the effluent bottle by way of a pump.

118. The composition of any one of claims 105-114, wherein the method comprises introducing a carbon source into the vessel.

119. The composition of claim 118, wherein the carbon source is continuously introduced into the vessel.

120. The composition of claim 118 or 119, wherein the carbon source is introduced into the vessel by way of a pump.

121. The composition of any one of claims 105-120, wherein the method comprises oxygenating the cell culture composition.

122. The composition of claim 121, wherein the cell culture composition is oxygenated by way of delivering compressed air into the vessel.

123. The composition of any one of claims 105-122, wherein the method comprises mixing the cell culture composition by way of an impeller.

124. The composition of any one of claims 105-123, wherein the cell culture product is a water-immiscible compound.

125. The composition of any one of claims 105-124, wherein the cell culture product is a compound that is inhibitory to the host cells.

126. The composition of any one of claims 105-125, wherein the cell culture product is a terpene.

127. The composition of claim 126, wherein the terpene is a C₅-C₄₀ terpene.

128. The composition of claim 127, wherein the terpene is a C₅-C₂₀ terpene.

129. The composition of claim 128, wherein the terpene is a C₁₀-C₁₅ terpene.

130. The composition of claim 126, wherein the terpene is a hemiterpene, monoterpene, sesquiterpene, diterpene, sesterterpene, triterpene, tetraterpene, or polyterpene.

131. The composition of claim 130, wherein the terpene is a monoterpene.

132. The composition of any one of claims 105-125, wherein the cell culture product is an isoprenoid.

133. The composition of claim 132, wherein the isoprenoid is a C₅-C₄₀ isoprenoid.

134. The composition of claim 133, wherein the isoprenoid is a C₅-C₂₀ isoprenoid.

135. The composition of claim 134, wherein the isoprenoid is a C₁₀-C₁₅ isoprenoid.

136. The composition of claim 135, wherein the isoprenoid is a hemiterpenoid, monoterpenoid, sesquiterpenoid, diterpenoid, sesterterpenoid, triterpenoid, tetraterpenoid, or polyterpenoid.

137. The composition of claim 136, wherein the isoprenoid is a monoterpenoid.

138. The composition of any one of claims 105-125, wherein the cell culture product is the cell culture product is abietadiene, anethole, amorphadiene, carene, carvacrol, creosol, cuminaldehyde, eugenol, α-farnesene, β-farnesene, farnesol, geranial, geraniol, geranylgeraniol, hinokitiol, isoprene, isoprenol, isopulegol, linalool, limonene, myrcene, nerolidol, ocimene, patchoulol, perillyl alcohol, α-pinene, β-pinene, sabinene, γ-terpinene, terpinolene, menthol, neral, nerol, eucalyptol, citronellol, citronellal, thujone, valencene, or a salvinorin.

139. The composition of claim 138, wherein the cell culture product is β-farnesene.

140. The composition of claim 138, wherein the cell culture product is myrcene.

141. The composition of claim 138, wherein the cell culture product is pinene.

142. The composition of claim 138, wherein the cell culture product is limonene.

143. The composition of claim 138, wherein the cell culture product is menthol.

144. The composition of claim 138, wherein the cell culture product is citronellal.

145. The composition of claim 138, wherein the cell culture product is citronellol.

146. The composition of claim 138, wherein the cell culture product is farnesol.

147. The composition of claim 138, wherein the cell culture product is terpinene.

148. The composition of claim 138, wherein the cell culture product is terpinolene.

149. The composition of claim 138, wherein the cell culture product is geraniol.

150. The composition of claim 138, wherein the cell culture product is linalool.

151. The composition of any one of claims 105-150, wherein the cell culture product has a minimum inhibitory concentration (MIC) in the host cells of greater than 5 mM.

152. The composition of any one of claims 105-150, wherein the cell culture product has a MIC in the host cells of from about 0.1 mM to about 5 mM.

153. The composition of claim 152, wherein the cell culture product has a MIC in the host cells of from about 0.1 mM to about 2.5 mM, optionally wherein the cell culture product has a MIC in the host cells of about 0.1 mM, about 0.15 mM, about 0.2 mM, about 0.25 mM, about 0.3 mM, about 0.35 mM, about 0.4 mM, about 0.45 mM, about 0.5 mM, about 0.55 mM, about 0.6 mM, about 0.65 mM, about 0.7 mM, about 0.75 mM, about 0.8 mM, about 0.85 mM, about 0.9 mM, about 0.95 mM, about 1 mM, about 1.25 mM, about 1.3 mM, about 1.35 mM, about 1.4 mM, about 1.45 mM, about 1.5 mM, about 1.55 mM, about 1.6 mM, about 1.65 mM, about 1.7 mM, about 1.75 mM, about 1.8 mM, about 1.85 mM, about 1.9 mM, about 1.95 mM, about 2 mM, about 2.25 mM, about 2.3 mM, about 2.35 mM, about 2.4 mM, about 2.45 mM, or about 2.5 mM.

154. The composition of any one of claims 105-153, wherein the water-immiscible solvent has a log(K_{d}) value of from about 1 to about 15, wherein the K_{d} is the partition coefficient for the cell culture product between the water-immiscible solvent and the cell culture composition.

155. The composition of claim 154, wherein the water-immiscible solvent has a log(K_{d}) value of from about 2 to about 3, optionally wherein the water-immiscible solvent has a log(K_{d}) value of about 2, about 2.1, about 2.2, about 2.3, about 2.4, about 2.5, about 2.6, about 2.7, about 2.8, about 2.9, or about 3.

156. The composition of any one of claims 105-155, wherein the water-immiscible solvent has a log(D) value of from about 1 to about 15.

157. The composition of any one of claims 105-156, wherein the water-immiscible solvent is an alcohol.

158. The composition of claim 157, wherein the water-immiscible solvent is a C₁₀-C₂₀ alcohol.

159. The composition of claim 157, wherein the water-immiscible solvent is a C₁₂-C₁₈ alcohol.

160. The composition of any one of claims 105-155, wherein the water-immiscible solvent is corn oil, sunflower oil, soybean oil, mineral oil, polyalphaolefin, dodecane, hexadecane, oleyl alcohol, butyl oleate, dibutyl phthalate, dodecanol, dioctyl phthalate, farnesene, or isopropyl myristate.

161. The composition of any one of claims 105-160, wherein the cell culture product is a fermentation product.

162. The method of any one of claims 105-161, wherein the cell culture composition is a fermentation composition.

163. The method of any one of claims 105-161, wherein the vessel is a fermentation vessel.
